(19) United States
(12) Patent Application Publication
(10) Pub. No.: US 2016/0115200 A1
(43) Pub. Date: Apr. 28, 2016

(54) New Use for JNK Inhibitor Molecules for Treatment of Various Diseases

(71) Applicant: XIGEN INFLAMMATION LTD., Limassol (CY)

(72) Inventors: Jean-Marc Combette, Saint Cergues (FR); Catherine Deloche, Genf (CH)

(21) Appl. No.: 14/890,859

(22) PCT Filed: Jun. 26, 2014
(86) PCT No.: PCT/EP2014/001737

§ 371 Date: Nov. 12, 2015

(30) Foreign Application Priority Data
Jun. 26, 2013 (EP) PCT/EP2013/001880

(51) Int. Cl. C07K 7/06

(52) U.S. Cl. CPC C07K 7/06

(57) ABSTRACT

The present invention relates to the use of novel JNK inhibitor molecules and their use in a method of treatment of the human or animal body by therapy.

The present invention relates to the field of enzyme inhibition, in particular to (poly-)peptide inhibitors of c-Jun amino terminal kinase (JNK). In particular, the present invention relates to using these JNK inhibitors in the treatment of various diseases.

The c-Jun amino terminal kinase (JNK) is a member of the stress-activated group of mitogen-activated protein (MAP) kinases. These kinases have been implicated in the control of cell growth and differentiation, and, more generally, in the response of cells to environmental stimuli. The JNK signal transduction pathway is activated in response to environmental stress and by the engagement of several classes of cell surface receptors. These receptors can include cytokine receptors, serpentine receptors and receptor tyrosine kinases. In mammalian cells, JNK has been implicated in biological processes such as oncogenic transformation and mediating adaptive responses to environmental stress. JNK has also been associated with modulating immune responses, including maturation and differentiation of immune cells, as well as effecting programmed cell death in cells identified for destruction by the immune system. This unique property makes JNK signaling a promising target for developing pharmacological intervention. Among several neurological disorders, JNK signaling is particularly implicated in ischemic stroke and Parkinson's disease, but also in other diseases as mentioned further below. Furthermore, the mitogen-activated protein kinase (MAPK) p38alpha was shown to negatively regulate the cell proliferation by antagonizing the JNK-c-Jun-pathway. The mitogen-activated protein kinase (MAPK) p38alpha therefore appears to be active in suppression of normal and cancer cell proliferation and, as a further, demonstrates the involvement of JNK in cancer diseases (see e.g. Hui et al, Nature Genetics, Vol 39, No. 6, June 2007). It was also shown, that c-Jun N-terminal Kinase (JNK) is involved in neuropathic pain produced by spinal nerve ligation (SNL), wherein SNL induced a slow and persistent activation of JNK, in particular JNK1, whereas p38 mitogen-activated protein kinase activation was found in spinal microglia after SNL, which had fallen to near basal level by 21 days (Zhuang et al., The Journal of Neuroscience, Mar. 29, 2006, 26(13):3551-3560)). In 2007 (Biochemica et Biophysica Acta, pp. 1341-1348), Johnson et al. discussed in a review the c-Jun kinase/stress-activated pathway, the involvement of JNK signalling in diseases such as the involvement in excitotoxicity of hippocampal neurons, liver ischemia, reperfusion, neurodegenerative diseases, hearing loss, deafness, neural tube birth defects, cancer, chronic inflammatory diseases, obesity, diabetes, in particular insulin-resistant diabetes, and proposed that it is likely that selective JNK inhibitors are needed for treatment of various diseases with a high degree of specificity and lack of toxicity.

Inhibition or interruption of the JNK signalling pathway is thus a promising approach in combating disorders strongly related to JNK signalling. However, there are only a few inhibitors of the JNK signaling pathway known so far.

Inhibitors of the JNK signaling pathway as already known in the prior art include e.g. upstream kinase inhibitors (for example, CEP-1347), small chemical inhibitors of JNK (SP600125 and AS601245), which directly affect kinase activity e.g. by competing with the ATP-binding site of the protein kinase, and peptide inhibitors of the interaction between JNK and its substrates (see e.g. Kuan et al., Current Drug Targets—CNS & Neurological Disorders, February 2005, vol. 4, no. 1, pp. 63-67; WO 2007/031280; all incorporated herewith by reference). WO 2007/031280 discloses small cell permeable fusion peptides, comprising a so-called TAT transporter sequence derived from the basic trafficking sequence of the HIV-TAT protein and an amino acid inhibitory sequence of IB1.

WO 2007/031280 discloses in particular two specific sequences, L-TAT-IB1 (GRKKRRQRRRPPRPKRPTTLNLFPQVPRSQD, herein SEQ ID NO: 196) and D-TAT-IB1 (dqsrpvqpflnlttprkprpprrrqrrkkrg; herein SEQ ID NO: 197), the latter being the retro-inverso sequence of L-TAT-IB1. Due to the HIV TAT derived transporter sequence, these fusion peptides are more efficiently transported into the target cells, where they remain effective until proteolytic degradation.

Since ATP independent peptide inhibitors of JNK are usually more specific inhibitors, they are frequently the first choice if it comes to inhibiting JNK. However, even the peptide inhibitors disclosed in WO 2007/031280 are not optimal for all purposes. For example, compound L-TAT-IB1 (herein SEQ ID NO: 196) which consists of L amino acids only, is quickly proteolytically degraded. In order to overcome this problem the inventors of WO 2007/031280 also suggested D-TAT-IB1 (herein SEQ ID NO: 197), which comprises D amino acids. To be more precise, D-TAT-IB1 exhibits the retro-inverso sequence of L-TAT-IB1. Incorporation of D-amino acids is made difficult by the fact that the change in stereochemistry may lead to a loss of function. The retro-inverso approach may be employed to reduce said risk because the use of i) only D-amino acids ii) but in the inverse peptide sequence may more likely yield an acceptable conformational analogue to the original peptide than incorporating one or more D-amino acids into the original sequence. In the case of WO 2007/031280 this approach resulted nevertheless in a significant decrease in inhibitory capacity in comparison to L-TAT-IB1 (see FIG. 4). Additionally, the retro-inverso peptide is extremely stable towards proteolytic digestion with the consequence that controlled digestions, for example in time sensitive experiments, are hardly possible.

JNK inhibitors have been discussed, proposed and successfully tested in the art as treatment for a variety of disease states. Already in 1997, Dickens et al. described the c-Jun amino terminal kinase inhibitor JIP-1 and proposed JIP-1 as candidate compounds for therapeutic strategies for the treatment of for example chronic myeloid leukaemia, in particular, in the context of Bcr-Abl caused transformation of pre-B-cells (Science; 1997; 277(5326):693-696).

In 2001, Bonny and co-workers published that cell-permeable peptide inhibitors of JNK confirm long term protection to pancreatic β-cells from IL-1β-induced apoptosis and may, thus, preserve β-cells in the autoimmune destruction in the course of diabetes (Diabetes, 50, 2001, p. 77-82).

Bonny et al. (Reviews in Neurosciences, 2005, p. 57-67) discussed also the inhibitory action of the JNK inhibitor D-JNKI-1 and other JNK inhibitors in the context of excitotoxicity, neuronal cell death, hypoxia, ischemia, traumatic brain damage, epilepsy, neurodegenerative diseases, apoptosis of neurons and inner ear sensory auditory cells etc.

In WO 98/49188 JIP-1 derived inhibitors of JNK signalling are proposed for the treatment of neurodegenerative diseases, such as Parkinson's disease or Alzheimer's disease; stroke and associated memory loss, autoimmune diseases such as arthritis; other conditions characterized by inflammation; malignancies, such as leukemias, e.g. chronic myelogenous leukemia (CML); oxidative damage to organs such as the liver and kidney; heart diseases; and transplant rejections.

Borsello et al. (Nat Med, 2003, (9), p. 1180-1186) published that a peptide inhibitor of c-Jun-N-terminal kinase protects against excitotoxicity and cerebral ischemia.

Assi et al. have published that another specific JNK-inhibitor, SP600125, targets tumor necrosis factor-α production and epithelial cell apoptosis in acute murine colitis. The authors concluded that inhibition of JNK is of value in human inflammatory bowel disease treatment (Immunology; 2006, 118(1):112-121).

In Kennedy et al. (Cell Cycle, 2003, 2(3), p. 199-201), the role of JNK signalling in tumor development is discussed in more detail.

Lee Yong Hee et al. (J Biol Chem 2003, 278(5), P. 2896-2902) showed that c-Jun N-terminal kinase (JNK) mediates feedback inhibition of the insulin signalling cascade and have proposed that inhibition of JNK signalling is a good therapeutic approach to reduce insulin resistance in diabetic patients.

Milano et al. (Am J Physiol Heart Circ Physiol 2007; 192(4): H1828-H1835) discovered that a peptide inhibitor of c-Jun NH₂-terminal kinase reduces myocardial ischemia-reperfusion injury and infarct size in vivo. The authors of said study used a peptide inhibitor, D-JNKI-I, a two domain peptide containing a 20 amino acid sequence of the minimal JNK-binding domain of islet-brain-1/JNK-interacting protein-1, linked to a 10 amino acid TAT sequence of the human immuno deficiency virus TAT protein that mediates intracellular translocation. The authors have concluded that a reduction in JNK activity and phosphorylation due to the presence of said inhibitor is important in the preservation of cardiac function in rats in the phase of ischemia and apoptosis.

A further group has published that small peptide inhibitors of JNKs protect against MPTP-induced nigral dopaminergic injury via inhibiting the JNK-signalling pathway (Pan et al., Laboratory investigation, 2010, 90, 156-167). The authors concluded that a peptide comprising residues 153-163 of murine JIP-1 fused to TAT peptide offers neuroprotection against MPTP injury via inhibiting the JNK-signalling pathway and provides a therapeutic approach for Parkinson's disease.

For hearing damage, Pirvola et al. (The Journal of Neuroscience, 2000, 20(1); 43-50) described the rescue of hearing, auditory hair cells and neurons by CEP-1347/KT7515, an inhibitor of c-Jun-N-terminal kinase activation. The authors suggested in general that therapeutic intervention in the JNK signalling cascade may offer opportunities to treat inner ear injuries. Treatment of hearing loss by means of administering JNK-inhibitory peptides is also disclosed for example in WO 03/103698.

For retinal diseases and age-related macula degeneration in particular, Roduit et al. (Apoptosis, 2008, 13(3), p. 343-353) have likewise suggested to use JNK-inhibition as therapeutic approach. Similar considerations relying on JNK-inhibition are disclosed for example in WO 2010/113753 for the treatment of age-related macular degeneration, diabetic macular edema, diabetic retinopathy, central exudative chorioretinopathy, angioid streaks, retinal pigment epithelium detachment, multifocal choroiditis, neovascular maculopathy, retinopathy of prematurity, retinitis pigmentosa, Leber's disease, retinal artery occlusion, retinal vein occlusion, central serous chorioretinopathy, retinal macroaneurysm, retinal detachment, proliferative vitreoretinopathy, Stargardt's disease, choroidal sclerosis, chorioderemia, vitelliform macular dystrophy, Oguchi's disease, fundus albipunctatus, retinitis punctata albescens, and gyrate atrophy of choroid and retina.

Zoukhri et al. (Journal of Neurochemistry, 2006, 96, 126-135) identified that c-Jun NH₂-terminal kinase mediates interleukin-1β-induced inhibition of lacrimal gland secretion. They concluded that JNK plays a pivotal role in IL-1β-mediated inhibition of lacrimal gland secretion and subsequent dry eye.

For uveitis, Touchard et al. (Invest Ophthalmol Vis Sci, 2010, 51(9); 4683-4693) have suggested to use D-JNKI 1 as effective treatment.

For IBD (inflammatory bowel disease) Roy et al. (World J Gastroenterol 2008, 14(2), 200-202) have highlighted the role of the JNK signal transduction pathway therein and have proposed to use peptidic JNK inhibitors for the treatment of said disease state.

Beckham et al (J Virol. 2007 July; 81(13):6984-6992) showed that the JNK inhibitor D-JNKI-1 is effective in protecting mice from viral encephalitis, and suggest thus JNK inhibition as promising and novel treatment strategy for viral encephalitis.

Palin et al. (Psychopharmacology (Berl). 2008 May; 197(4):629-635) used the same JNK inhibitor, D-JNKI-1, and found that pre-treatment with D-JNKI-1 (10 ng/mouse), but not D-TAT, significantly inhibited all three indices of sickness induced by central TNFalpha and suggested that JNK inhibition as means for treating major depressive disorders that develop on a background of cytokine-induced sickness behaviour.

In WO 2010/151638 treatment of the neurodegenerative disease spinal muscular atrophy by way of JNK inhibition was proposed.

The above introductory section highlights on the basis of selected publications the usefulness of JNK inhibitors in the treatment of various diseases. Thus, there is a constant need in the art for JNK inhibitors for use in the treatment of human (and animal) diseases.

Thus, the problem to be solved by the present invention was to provide further (peptide) inhibitors of JNK for the treatment of specific diseases.

The object of the present invention is solved by the inventor by means of the subject-matter set out below and in the appended claims.

### BRIEF DESCRIPTION OF THE FIGURES

In the following a brief description of the appended figures will be given. The figures are intended to illustrate the present invention in more detail. However, they are not intended to limit the subject matter of the invention in any way.

FIG. 1: Illustration of the inhibitory efficacy of several JNK inhibitors according to the present invention, which was investigated by in vitro AlphaScreen assay (Amplified Luminescence Proximity Homogeneous-Screen Assay).
FIG. 1A: Inhibition of JNK1 by SEQ ID NOs: 193, 2, 3, 5, 6, and 7.
FIG. 1B: Inhibition of JNK2 by SEQ ID NOs: 193, 2, 3, 5, 6, and 7.
FIG. 1C: Inhibition of JNK3 by SEQ ID NOs: 193, 2, 3, 5, 6, and 7.

FIG. 2: Table illustrating the inhibitory efficacy of several JNK inhibitors (SEQ ID NOs: 193, 2, 3, 5, 6, and 7) according to the present invention. Given are the IC50 values in the nM range, the respective standard error of the mean and the number of experiments performed (n).

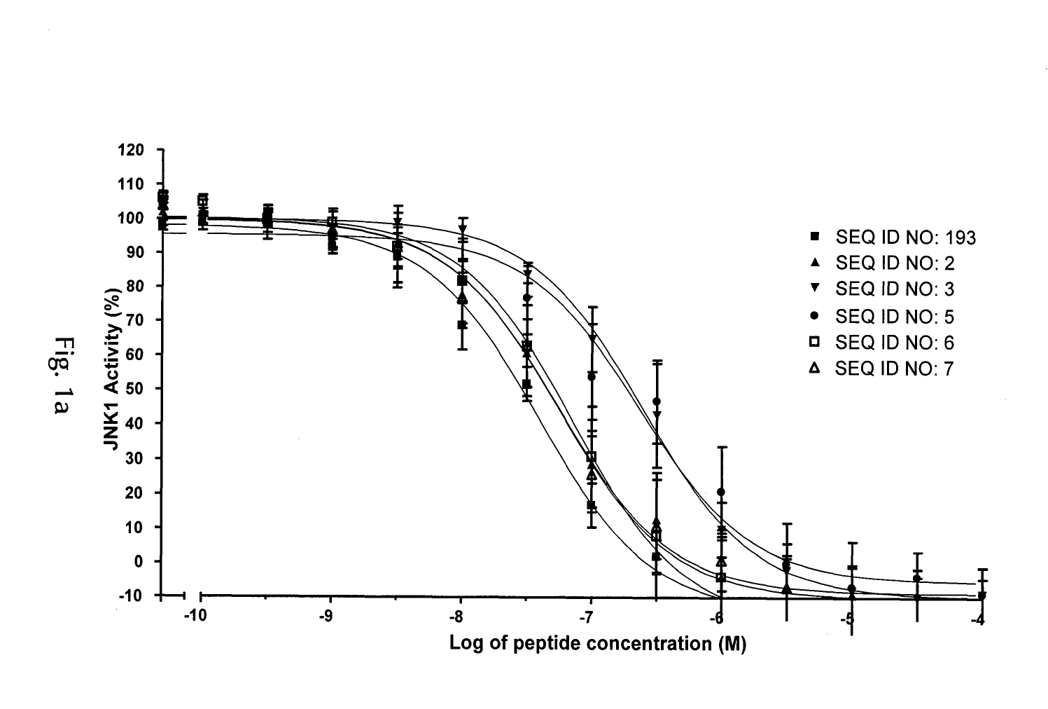

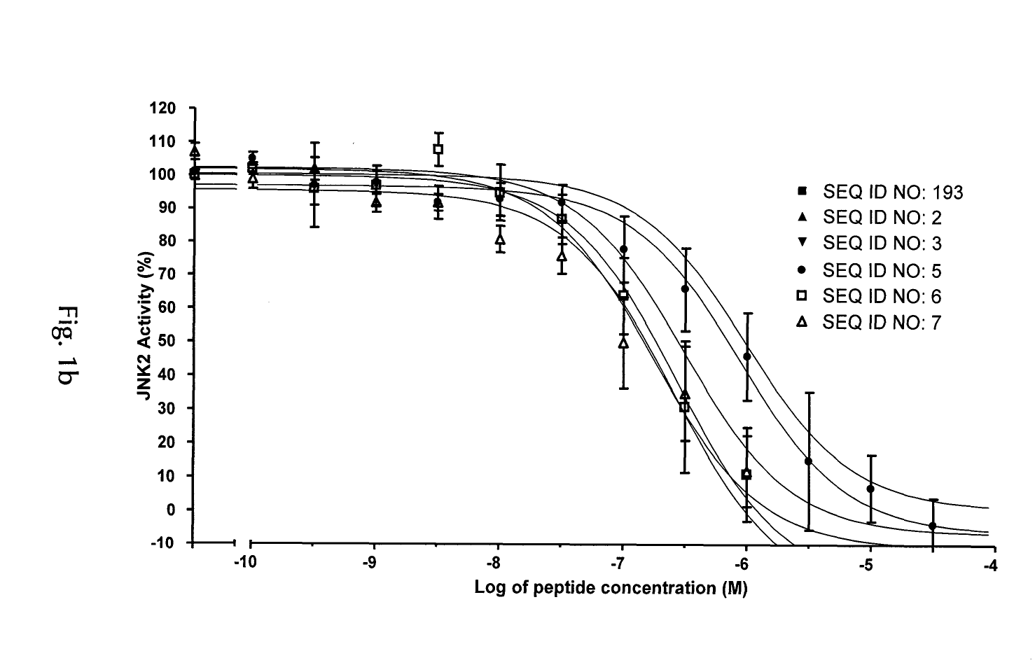

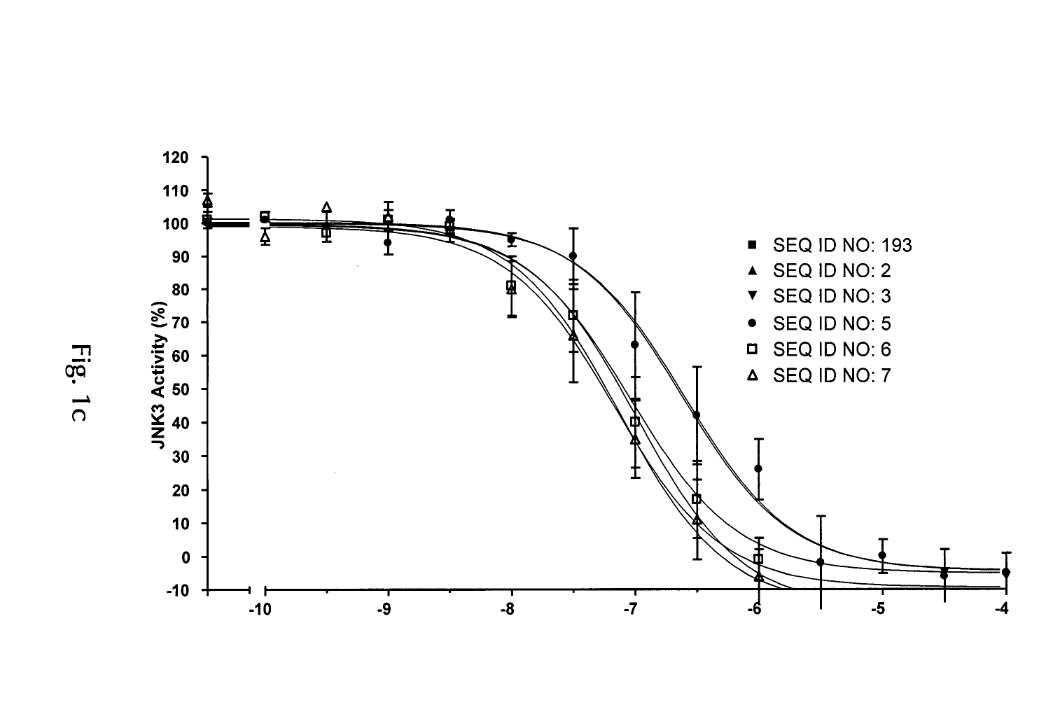

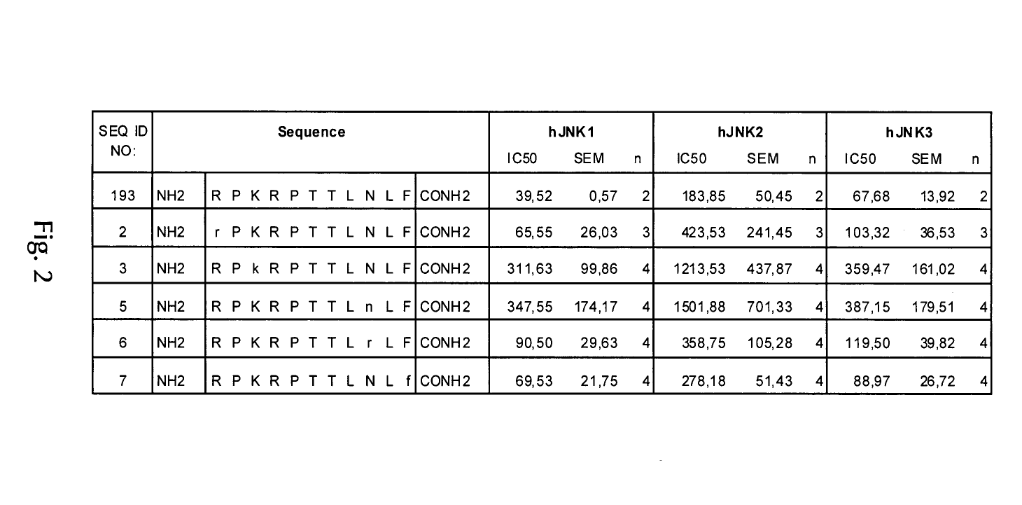

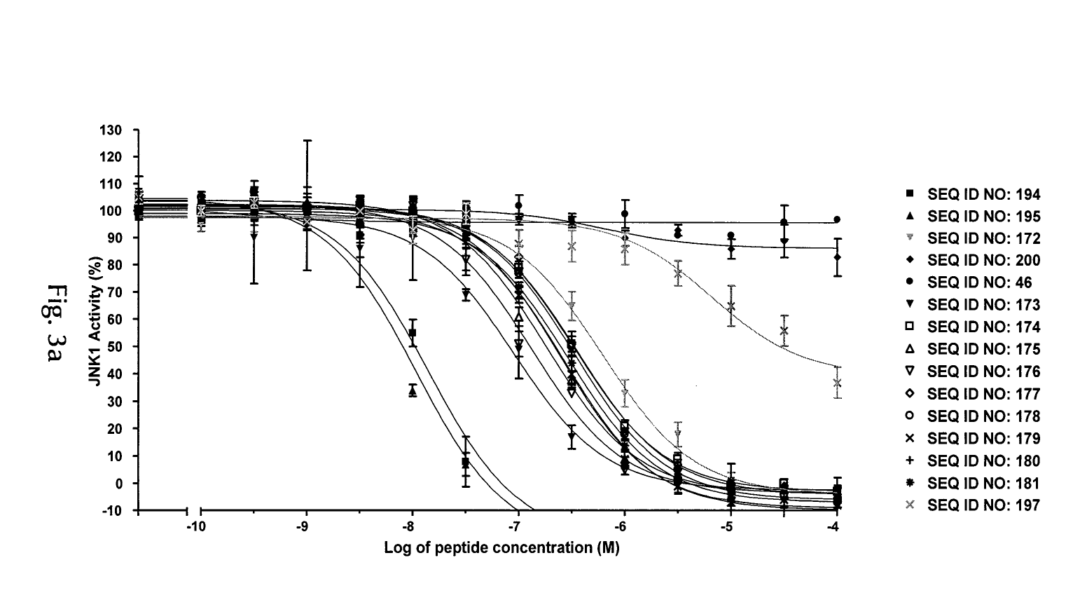

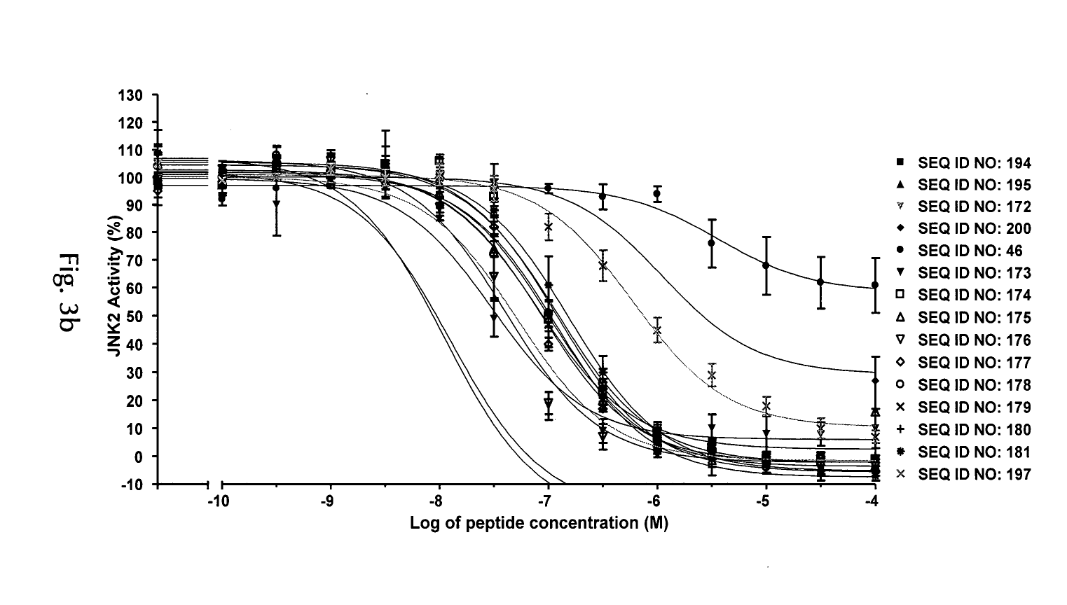

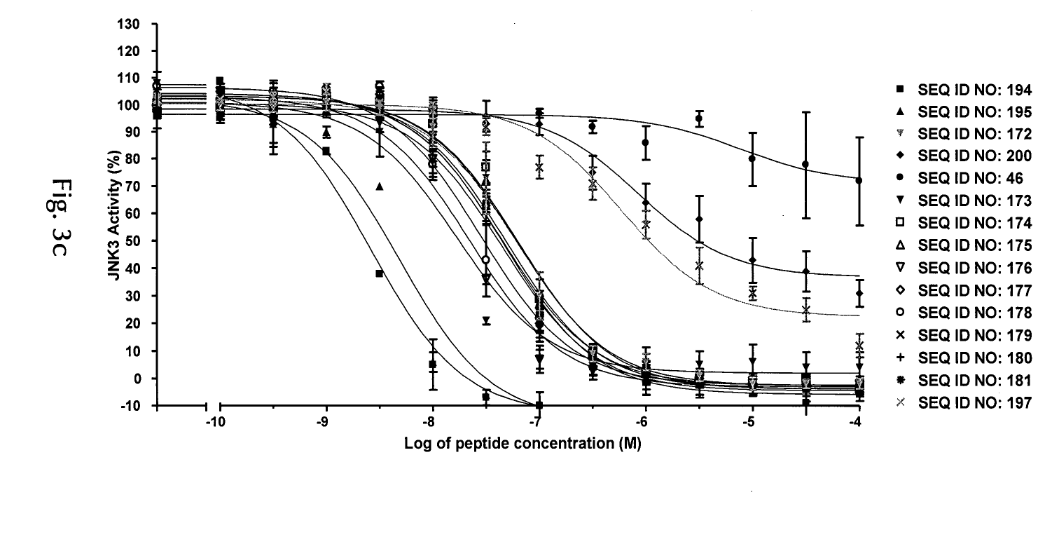

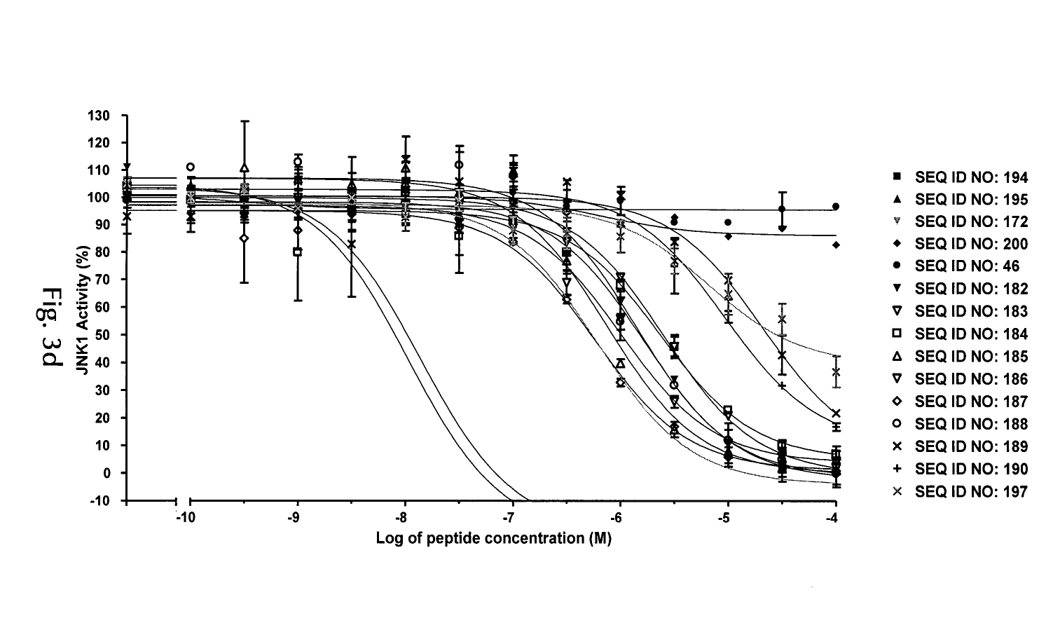

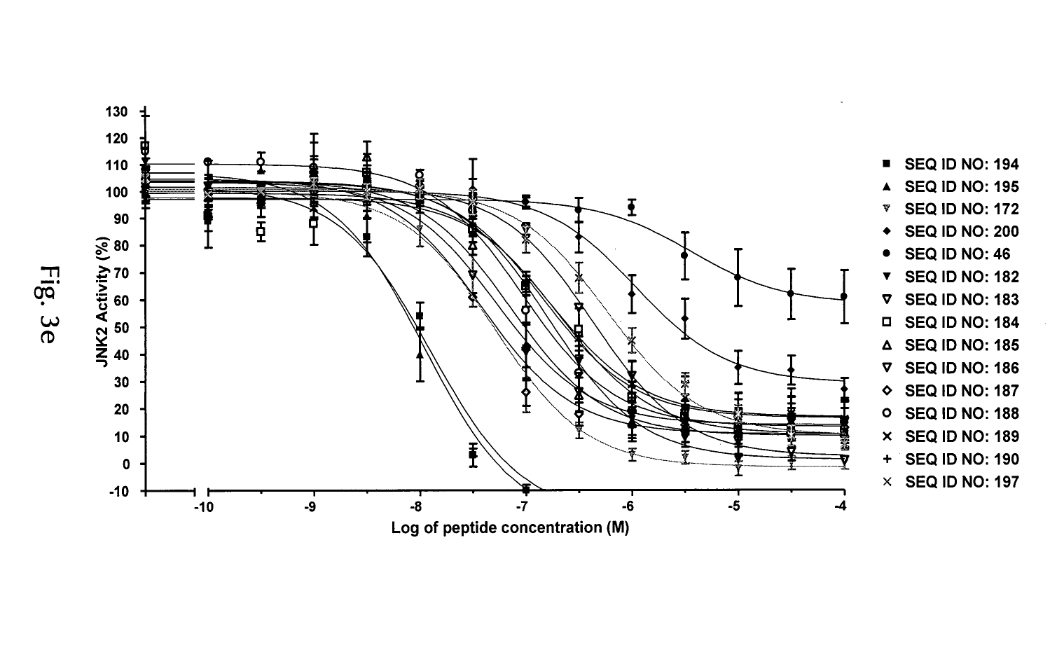

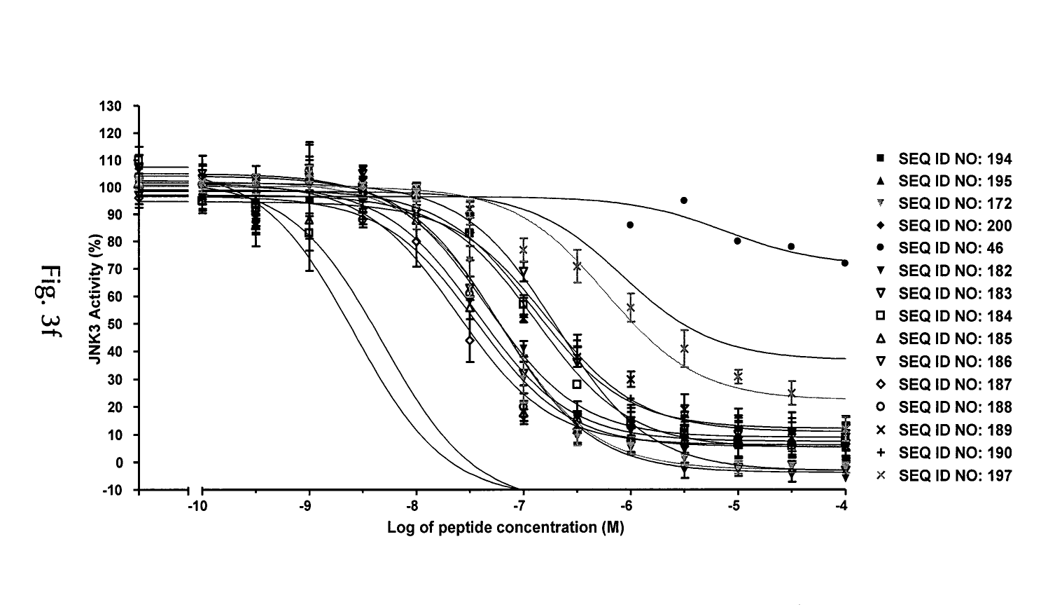

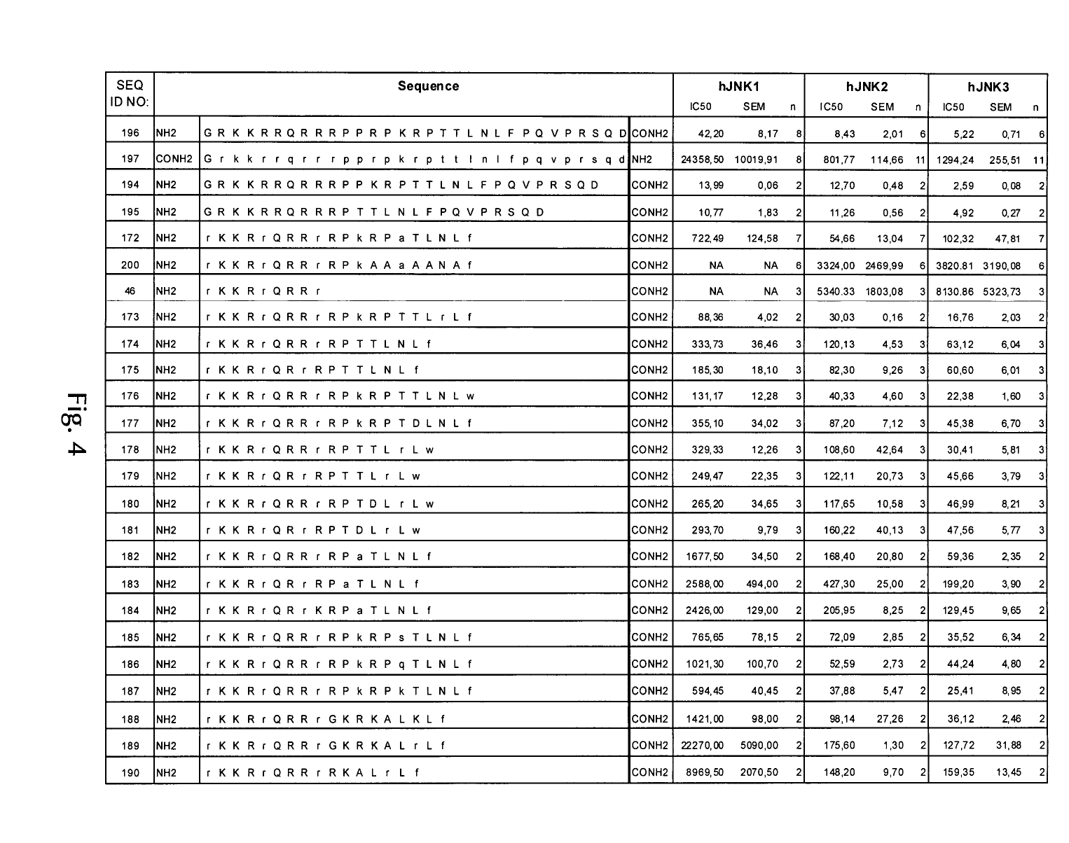

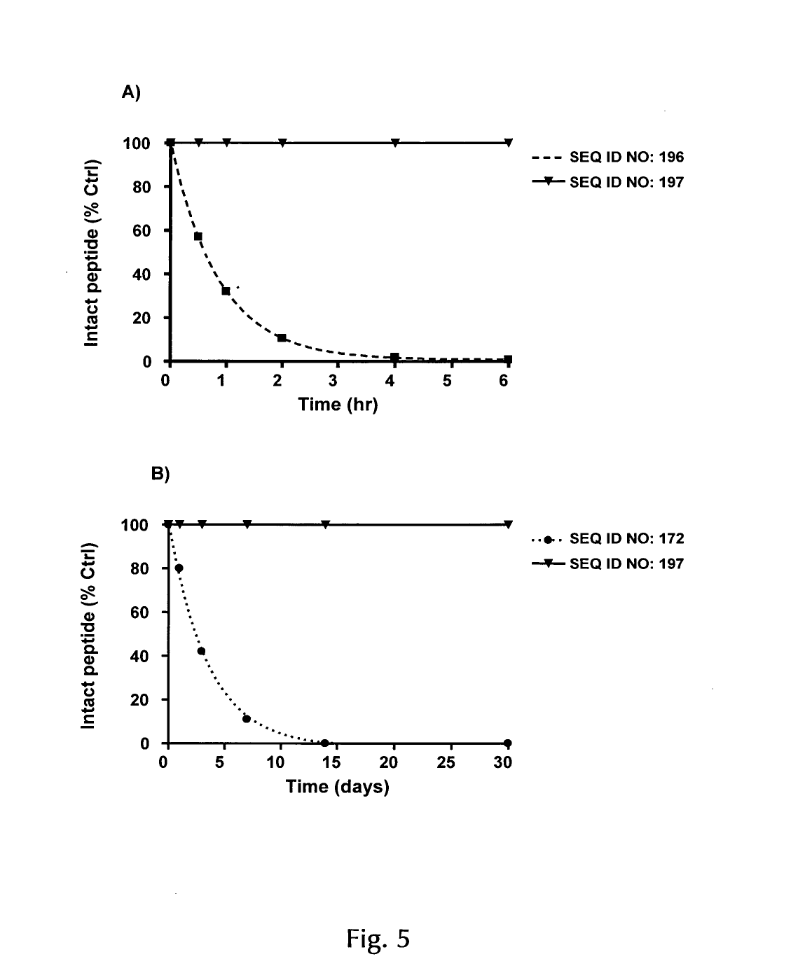

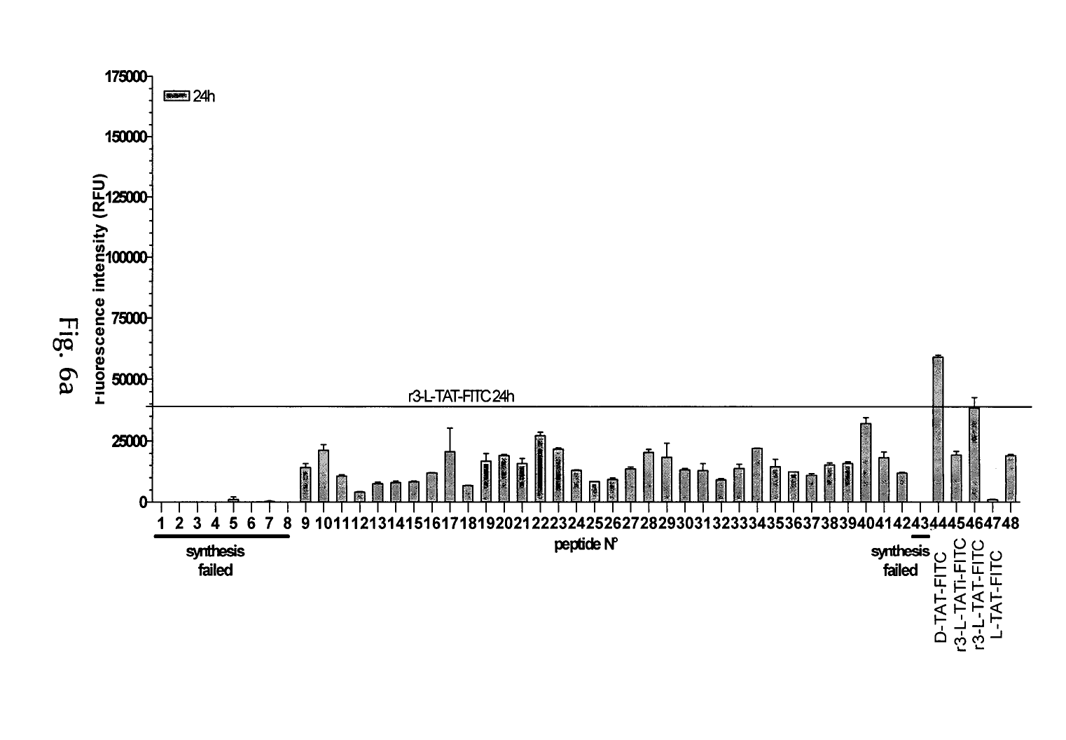

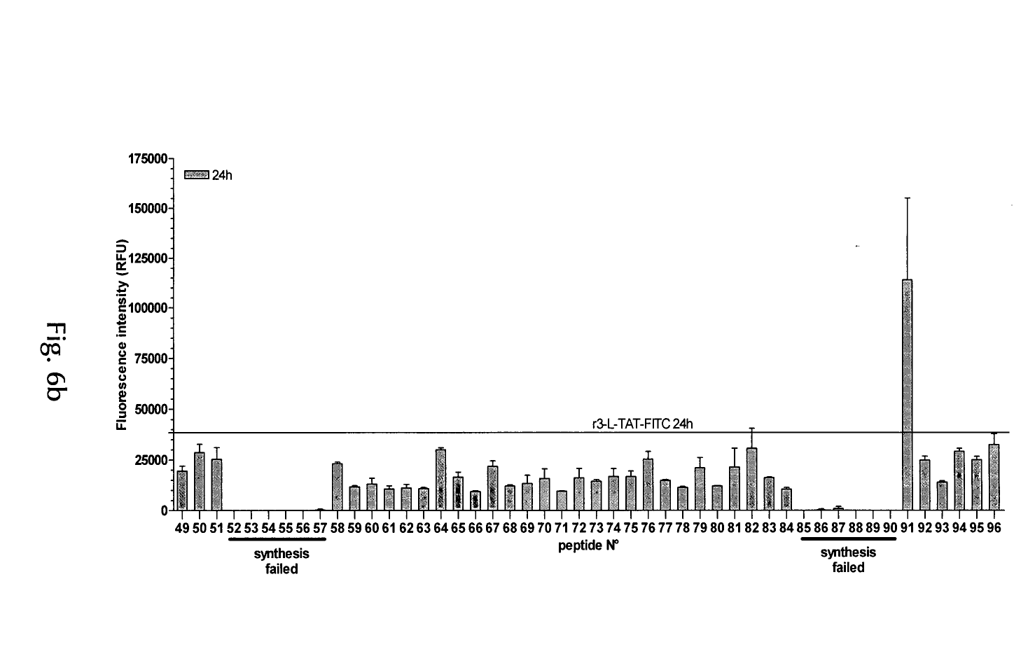

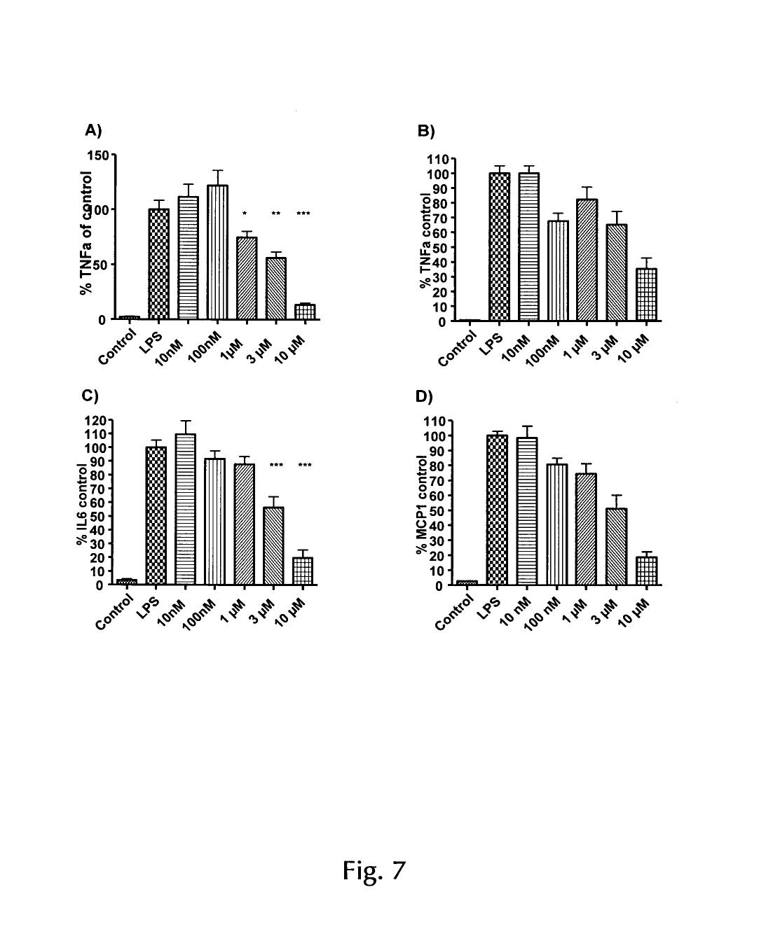

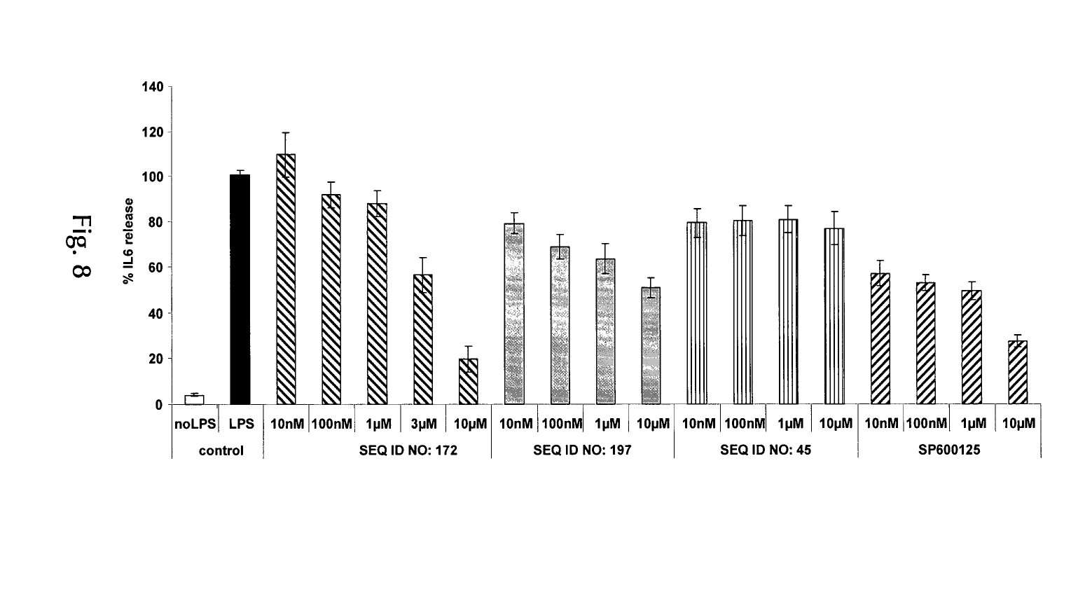

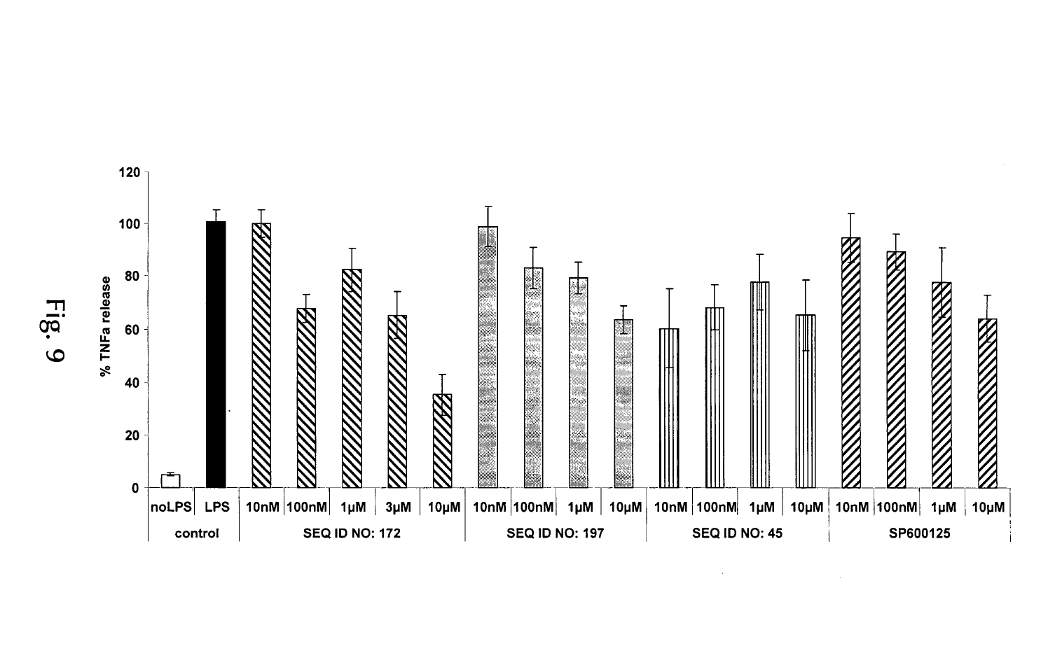

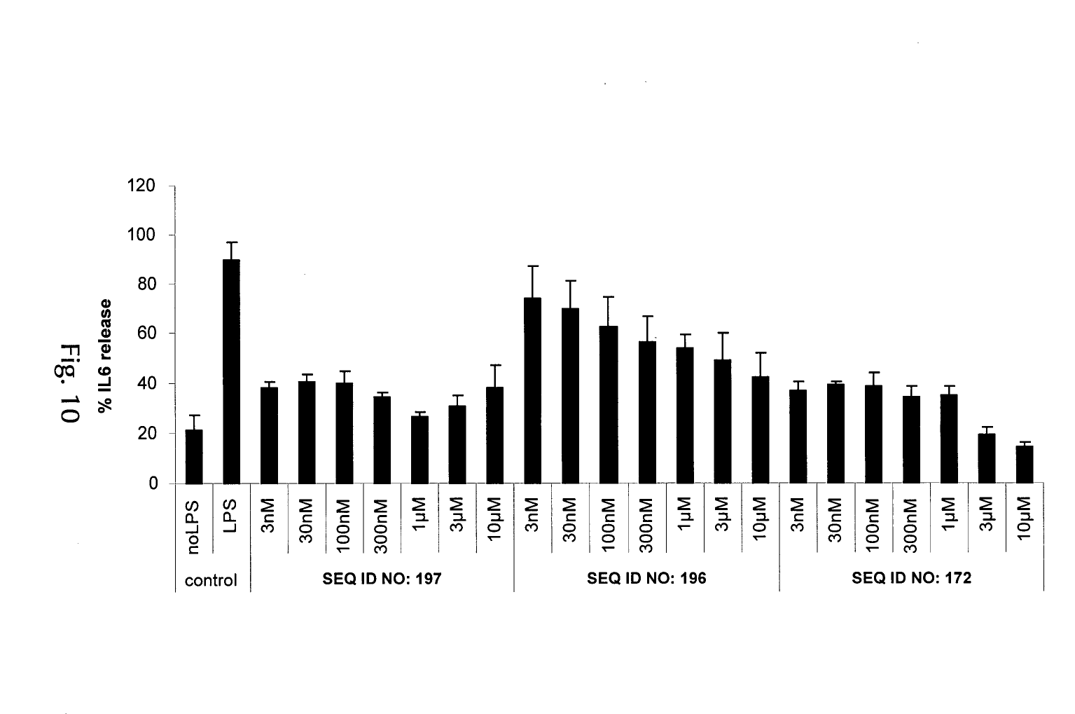

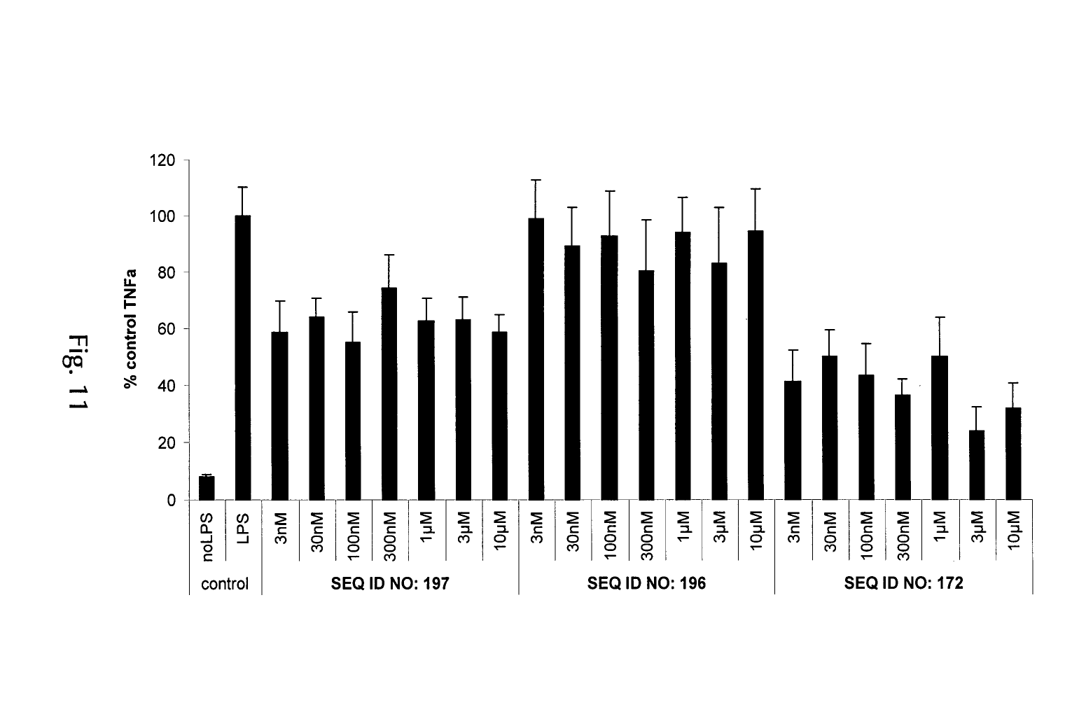

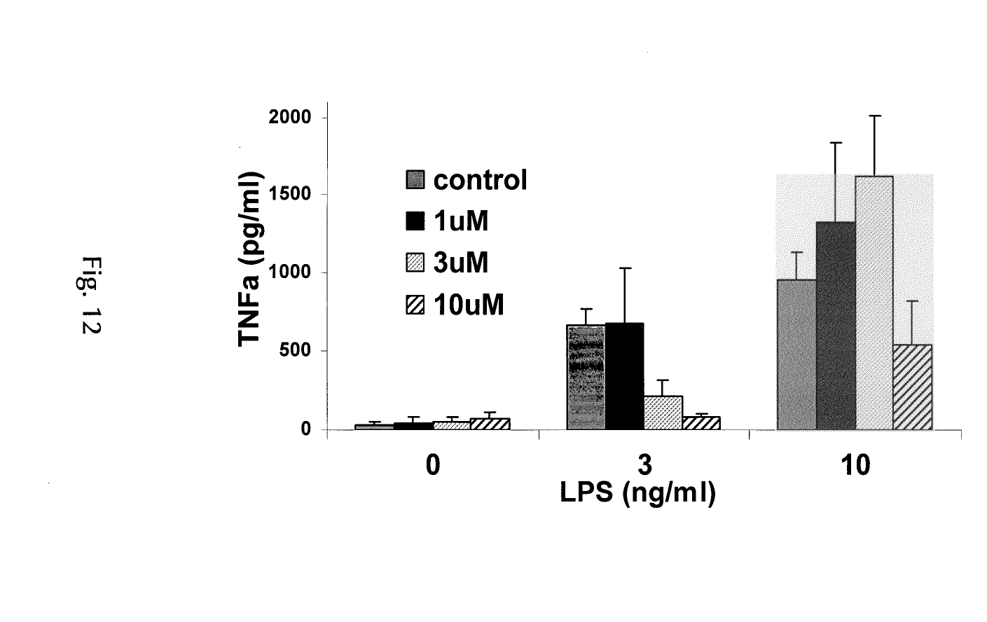

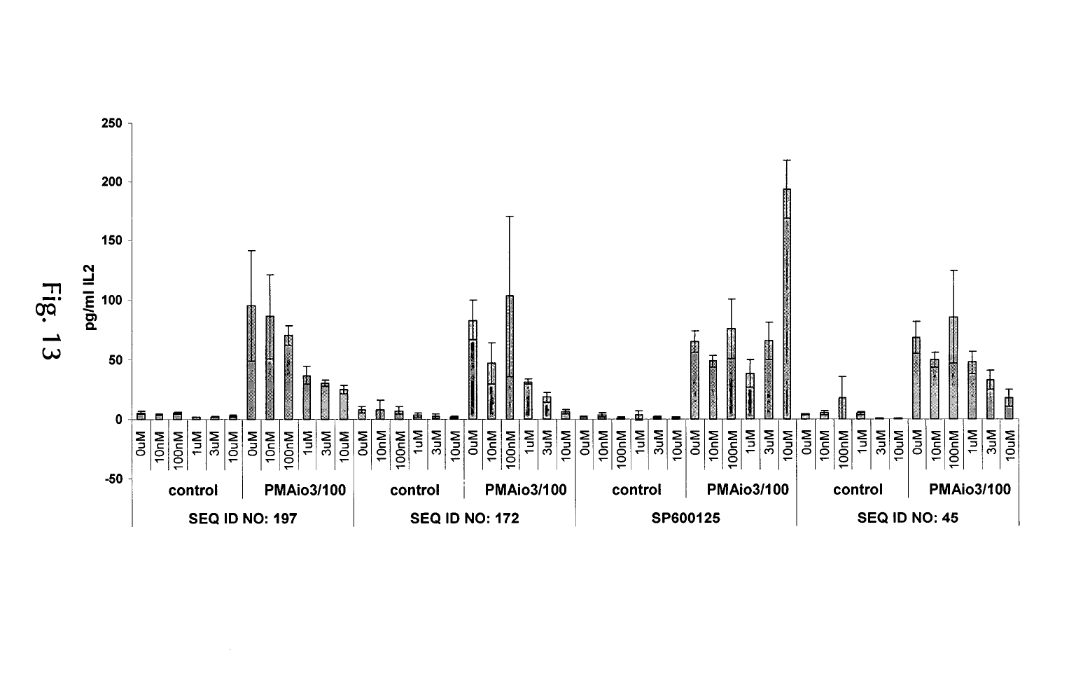

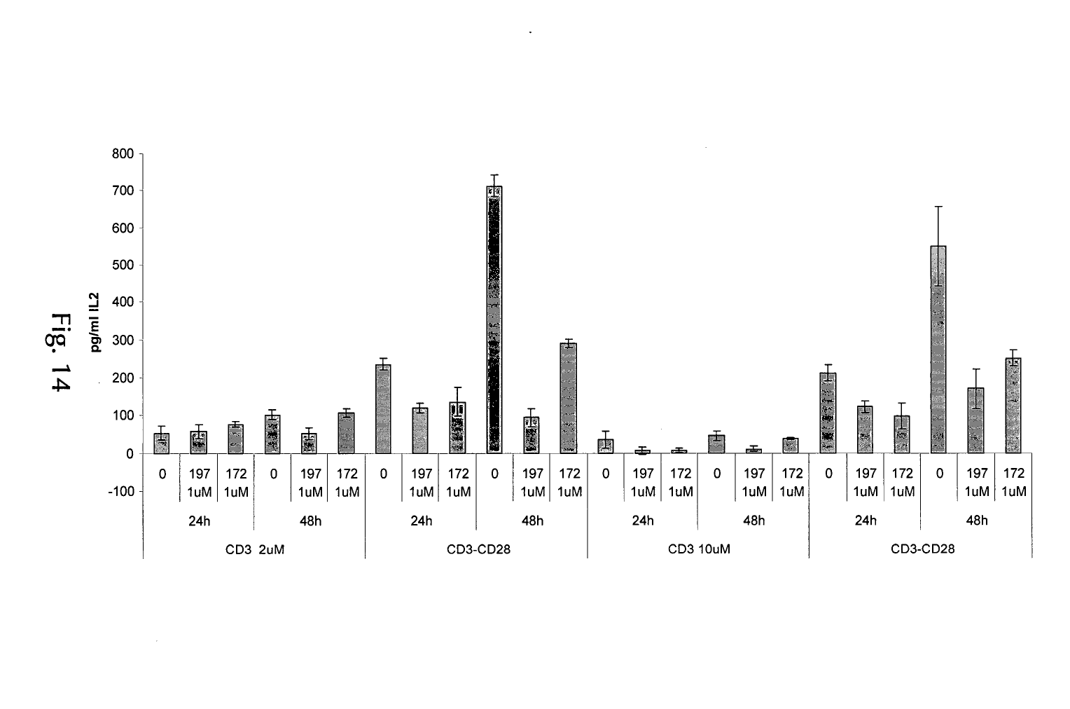

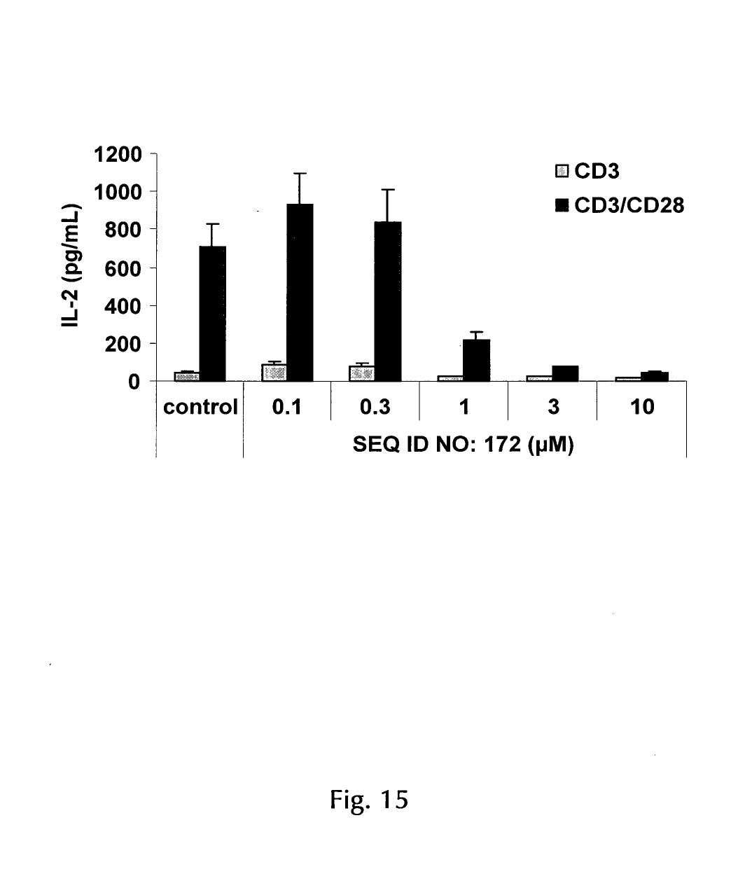

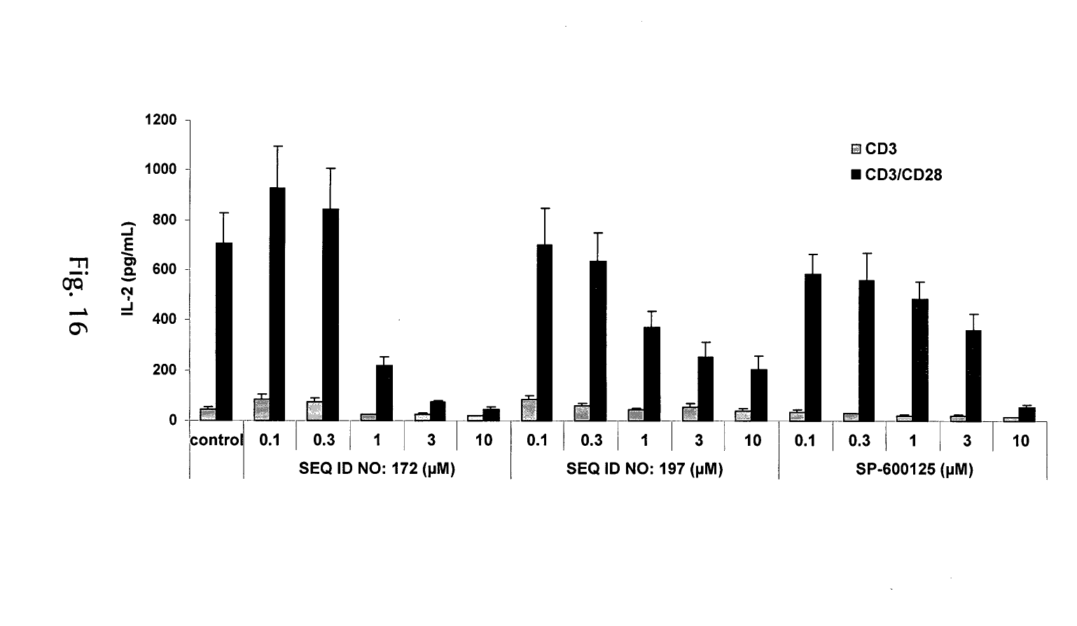

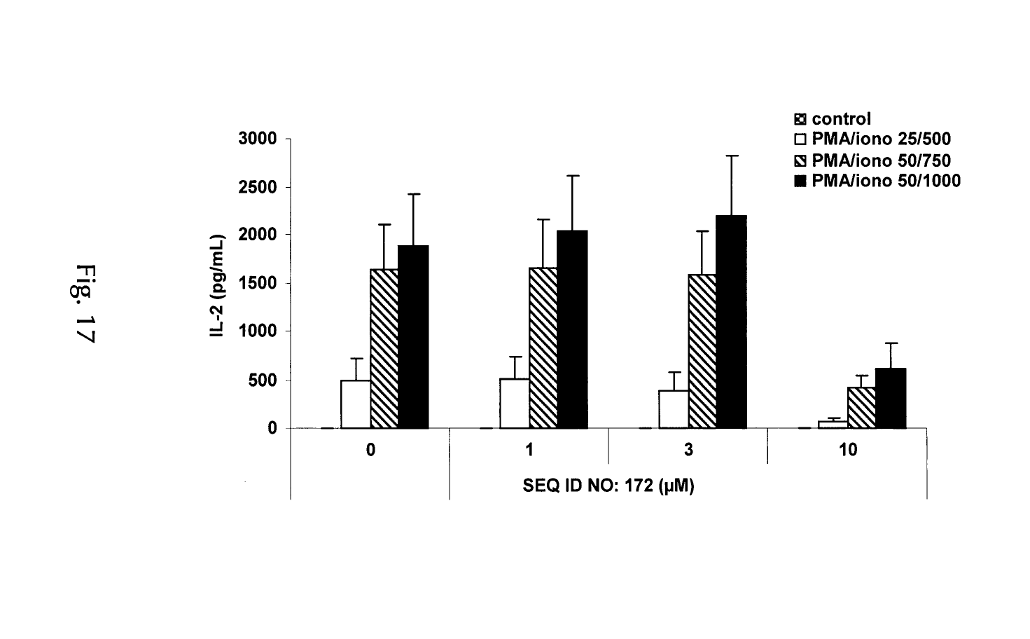

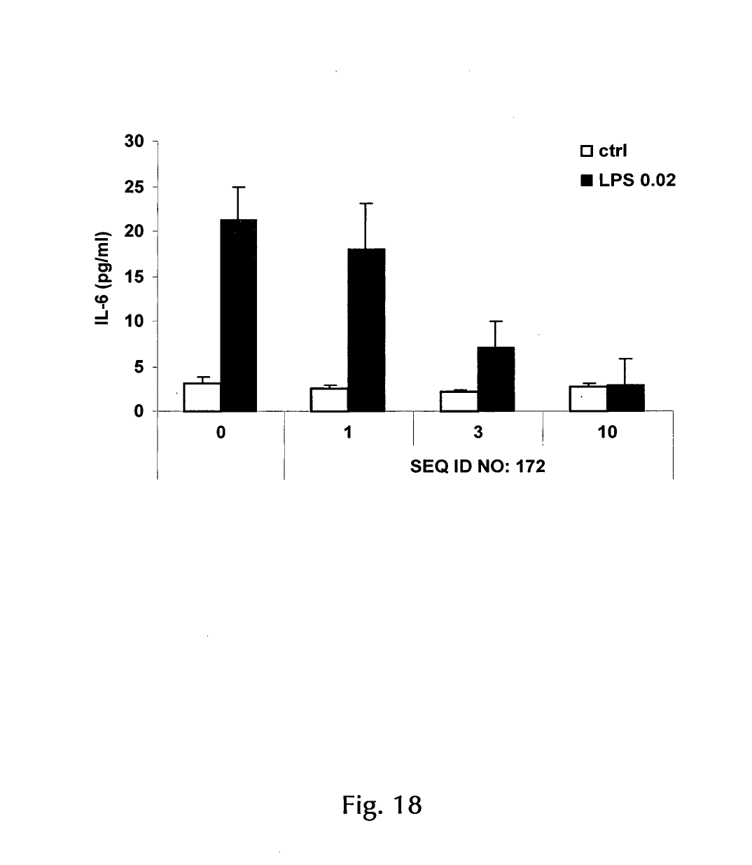

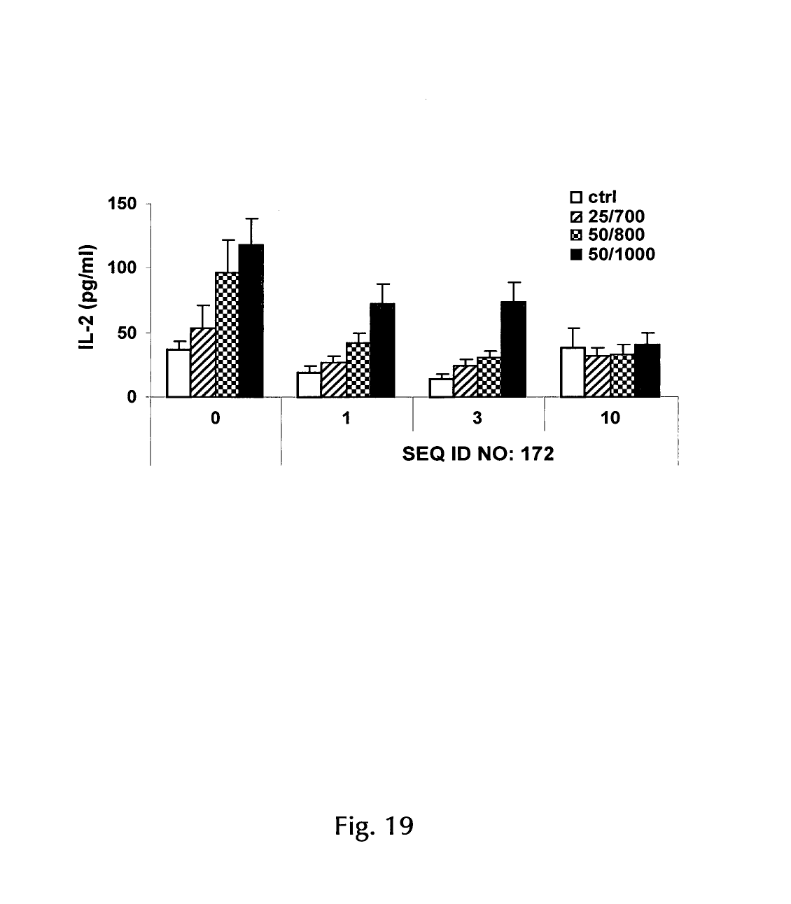

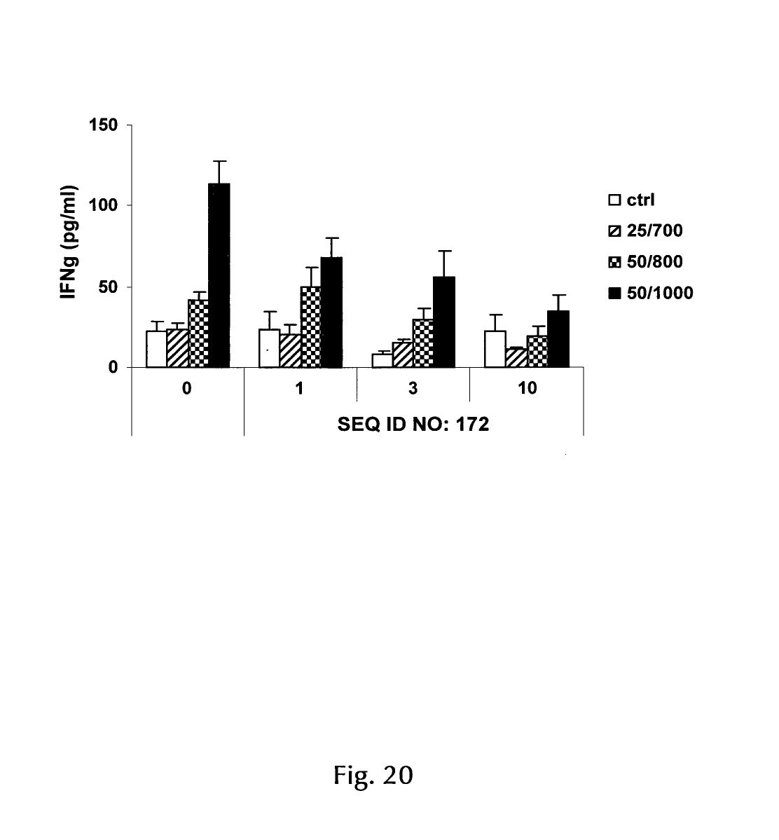

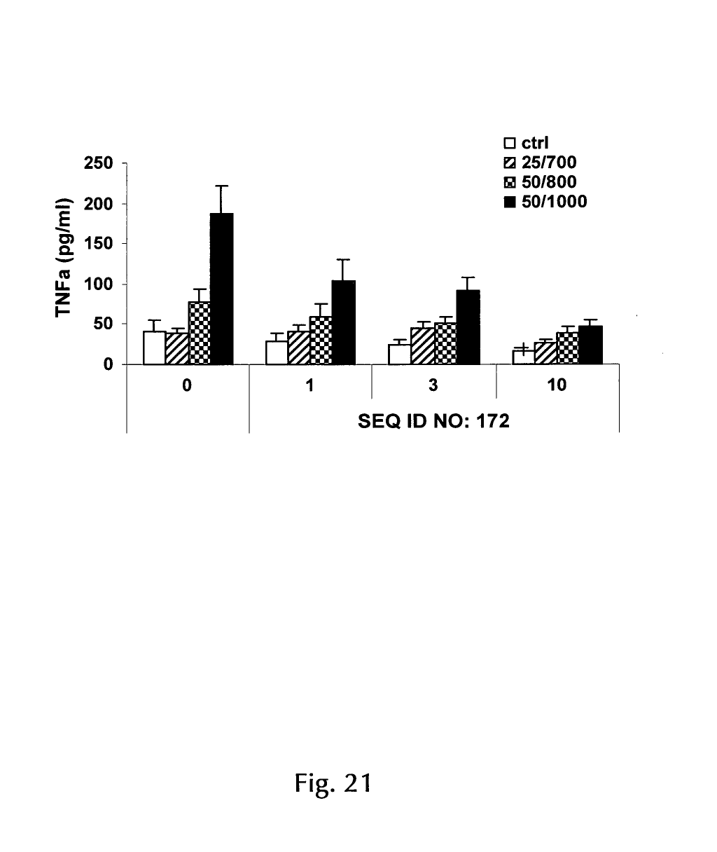

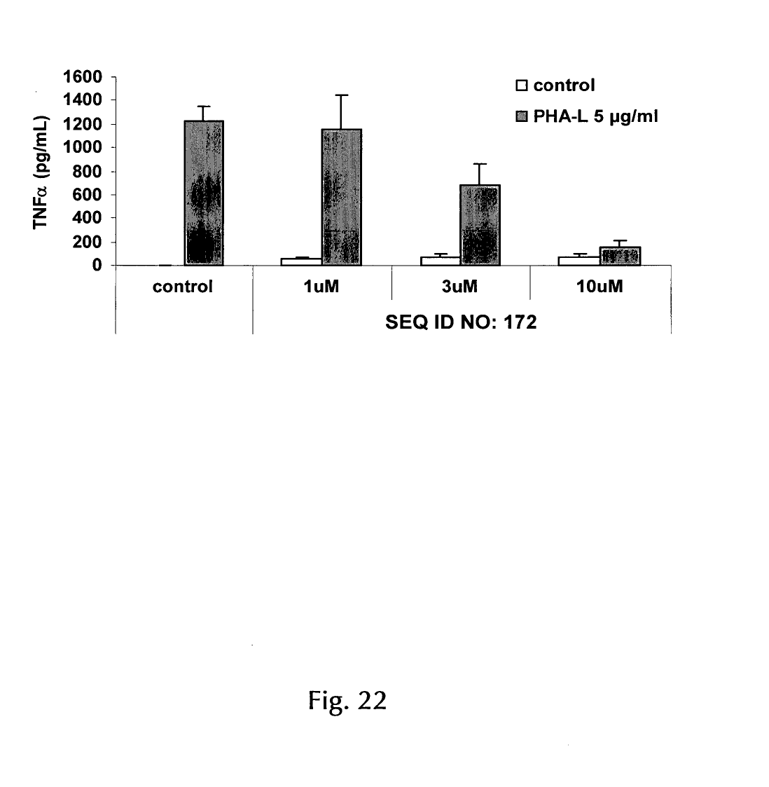

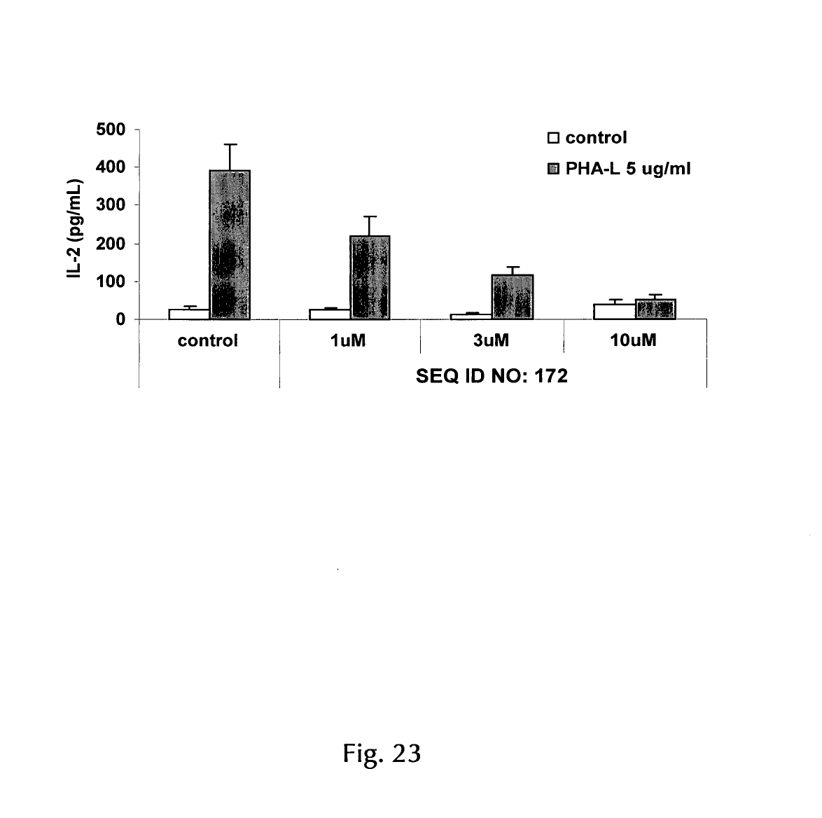

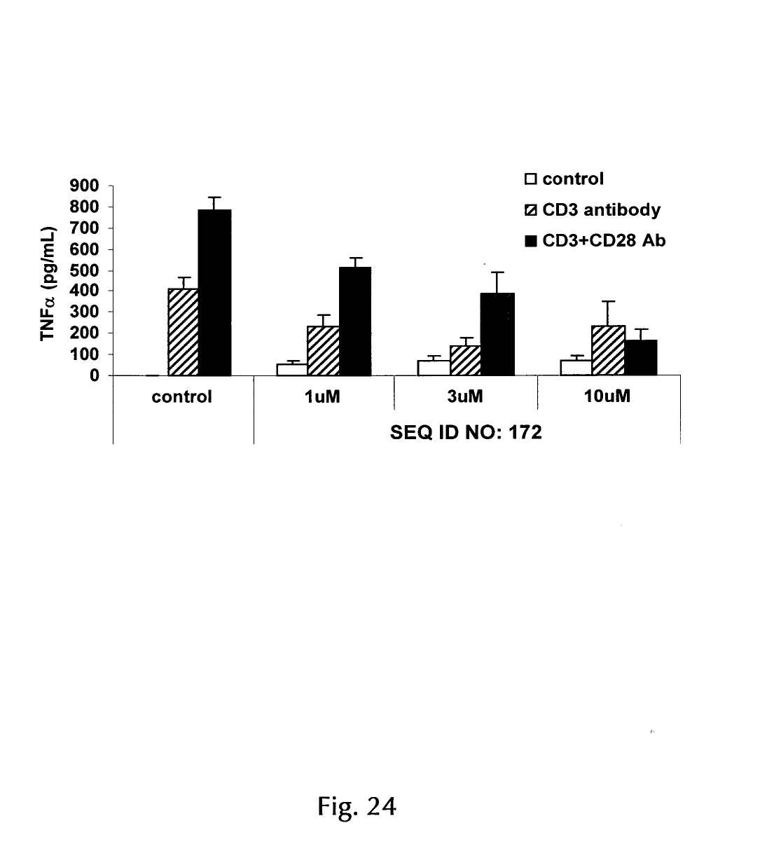

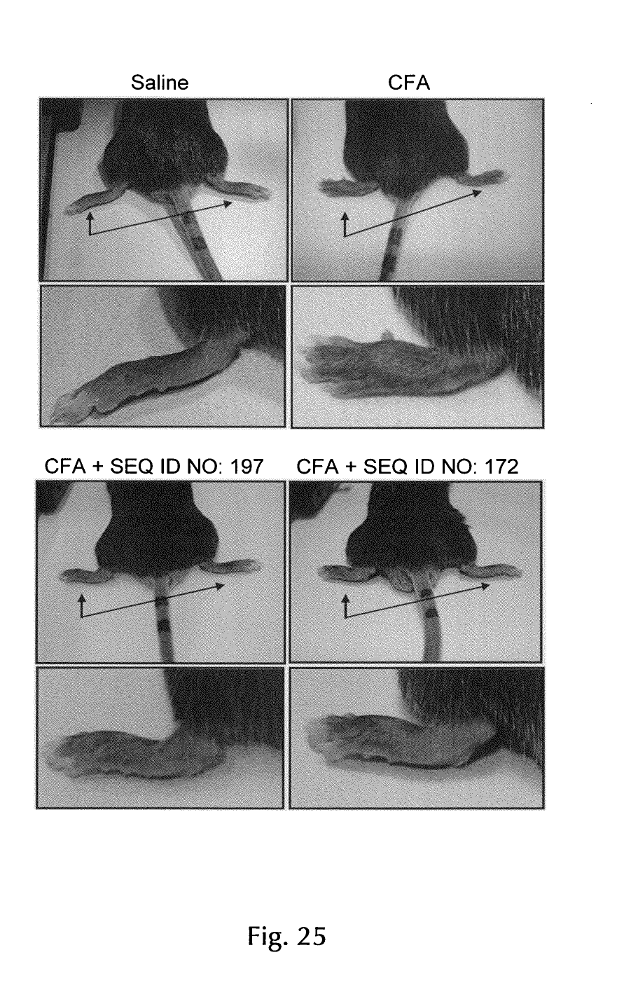

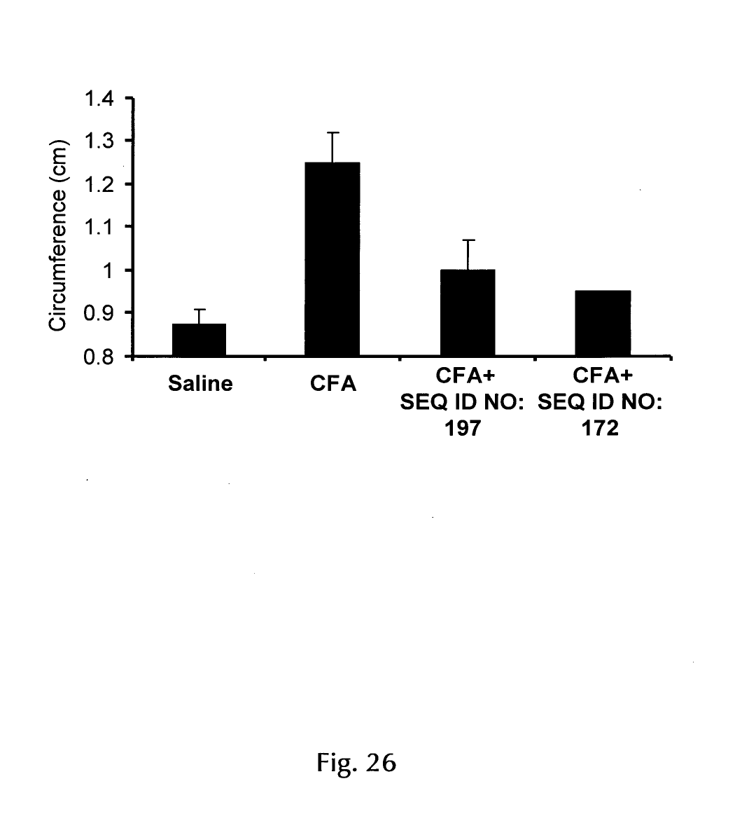

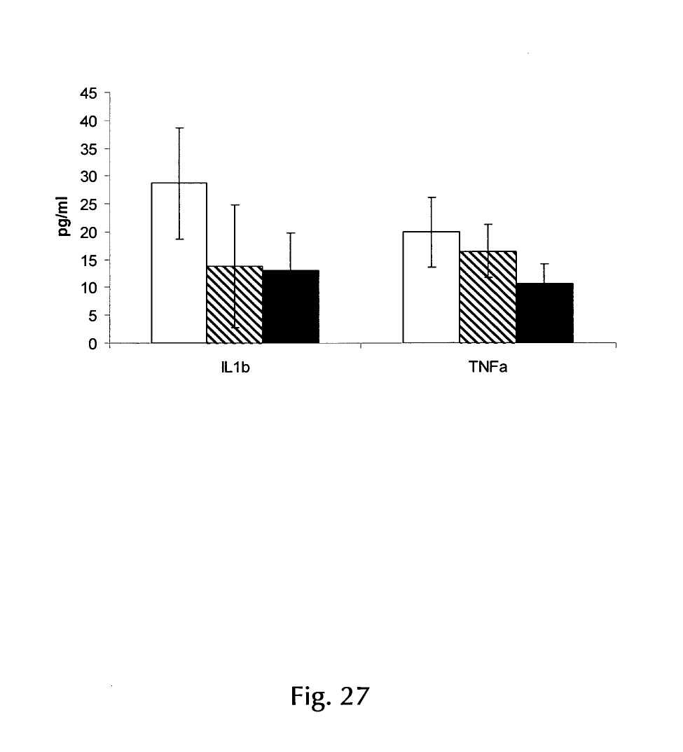

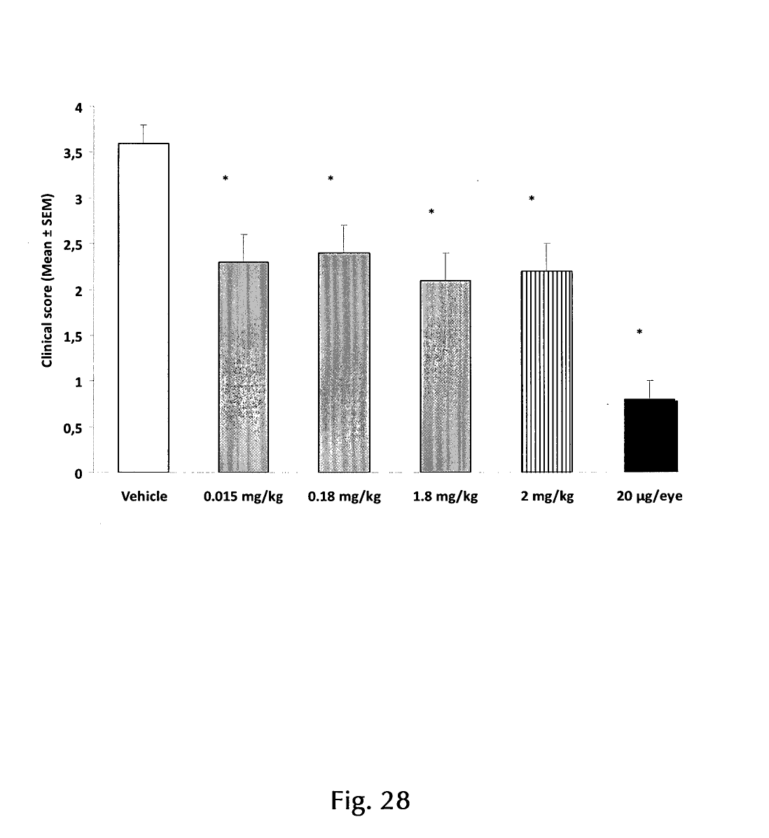

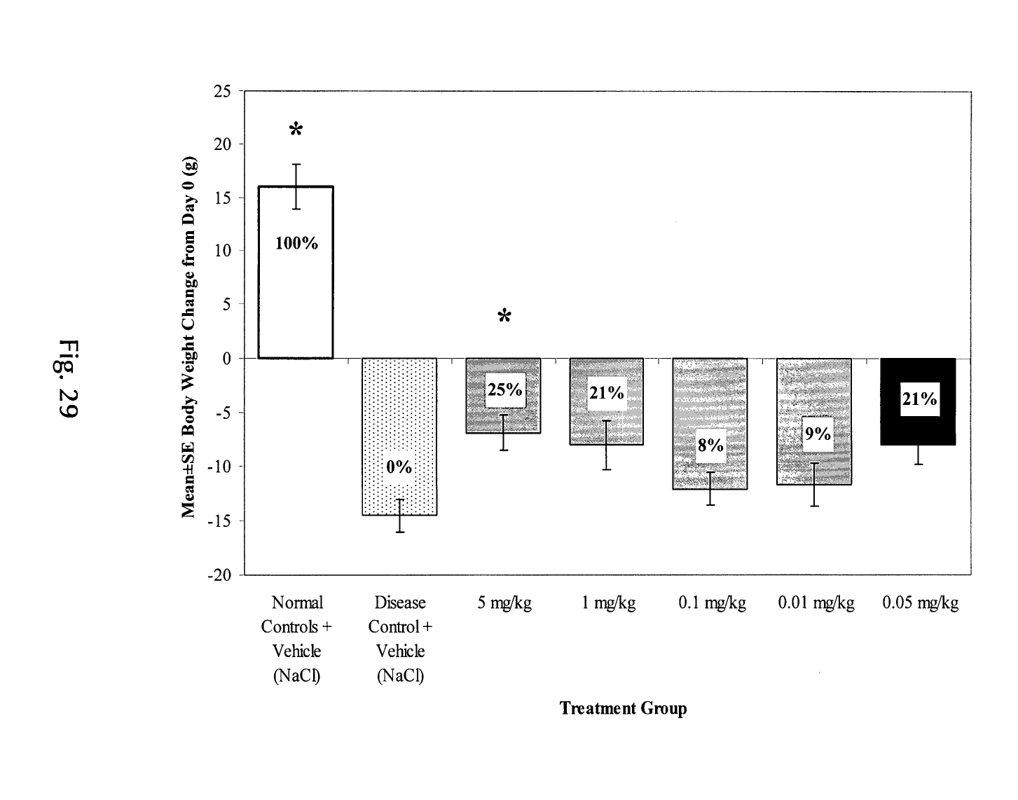

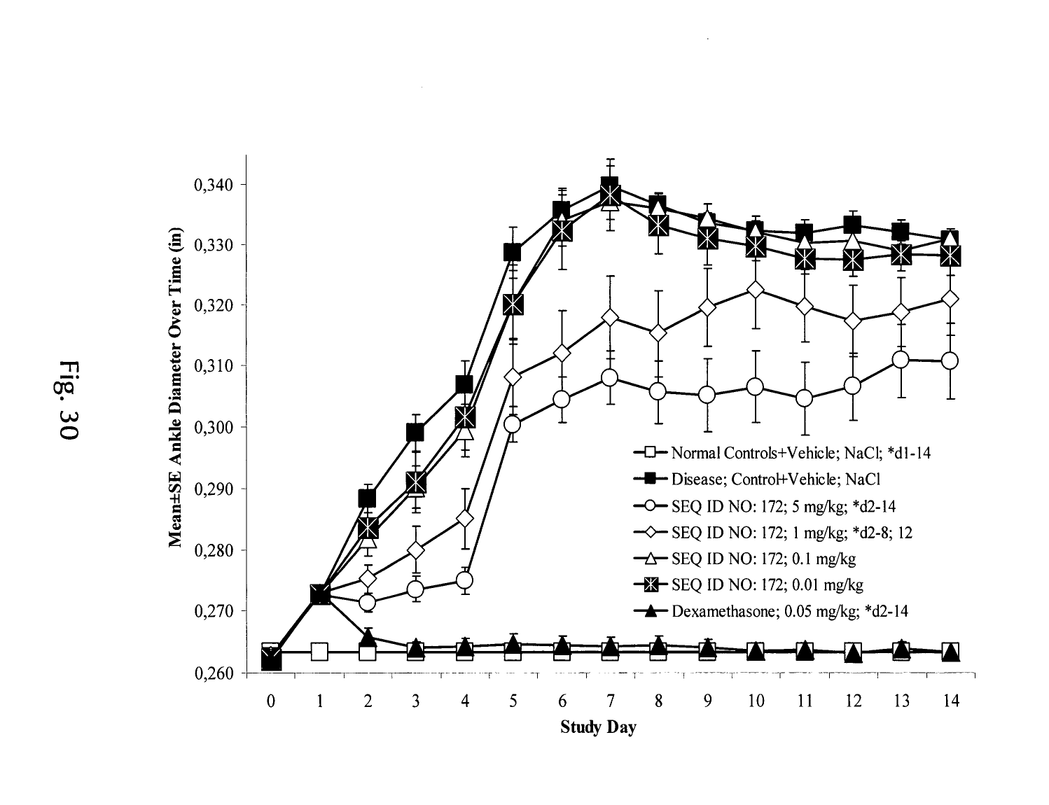

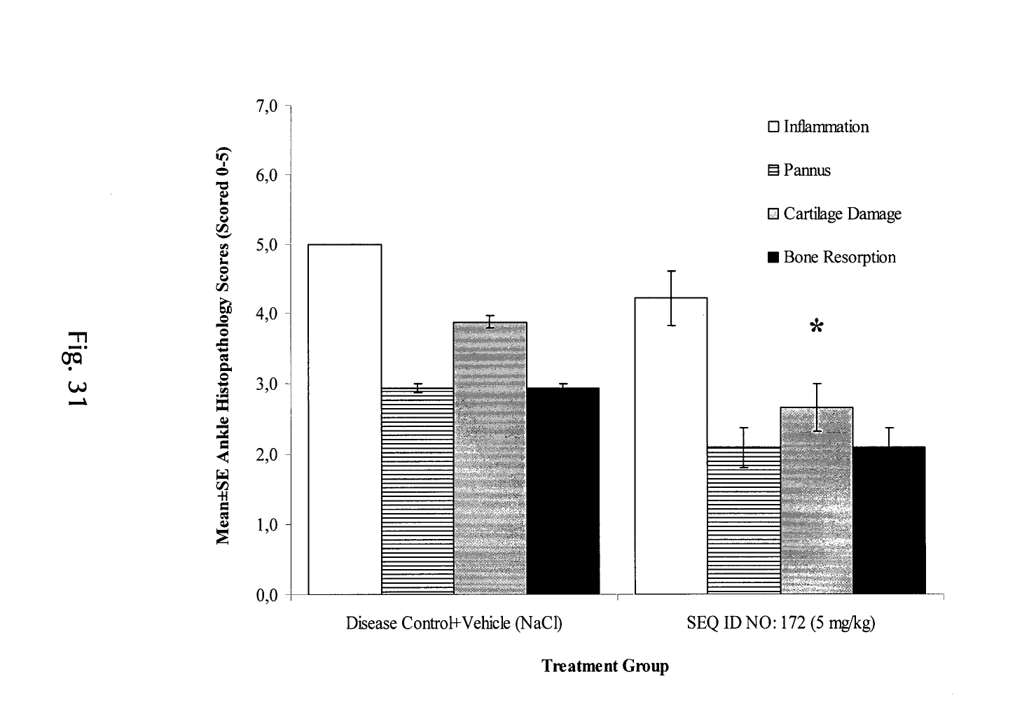

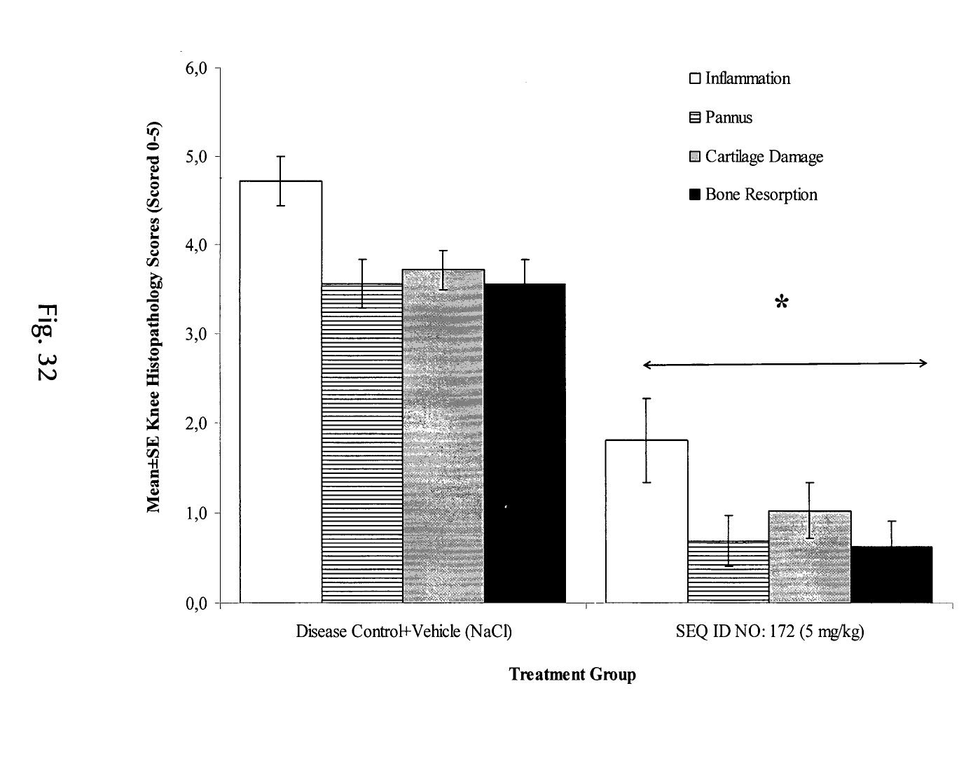

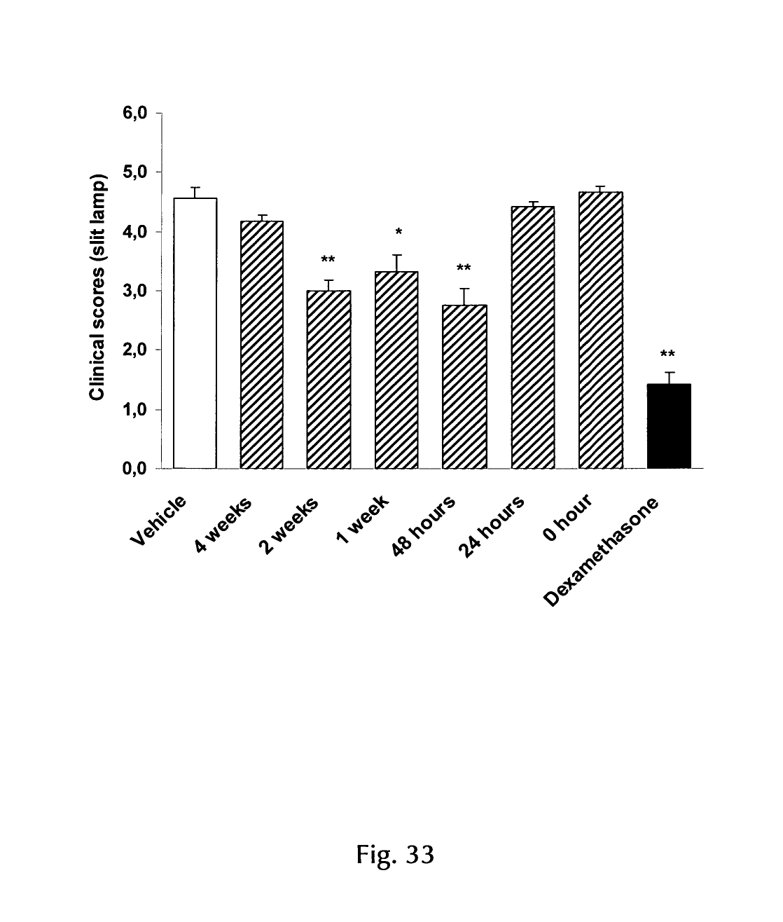

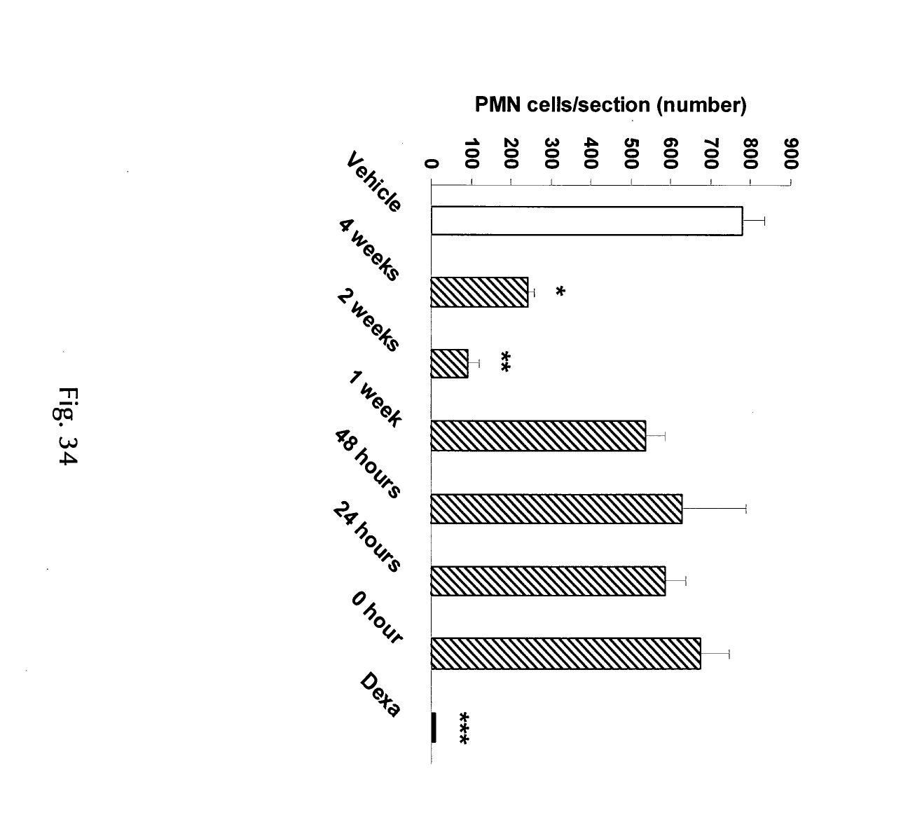

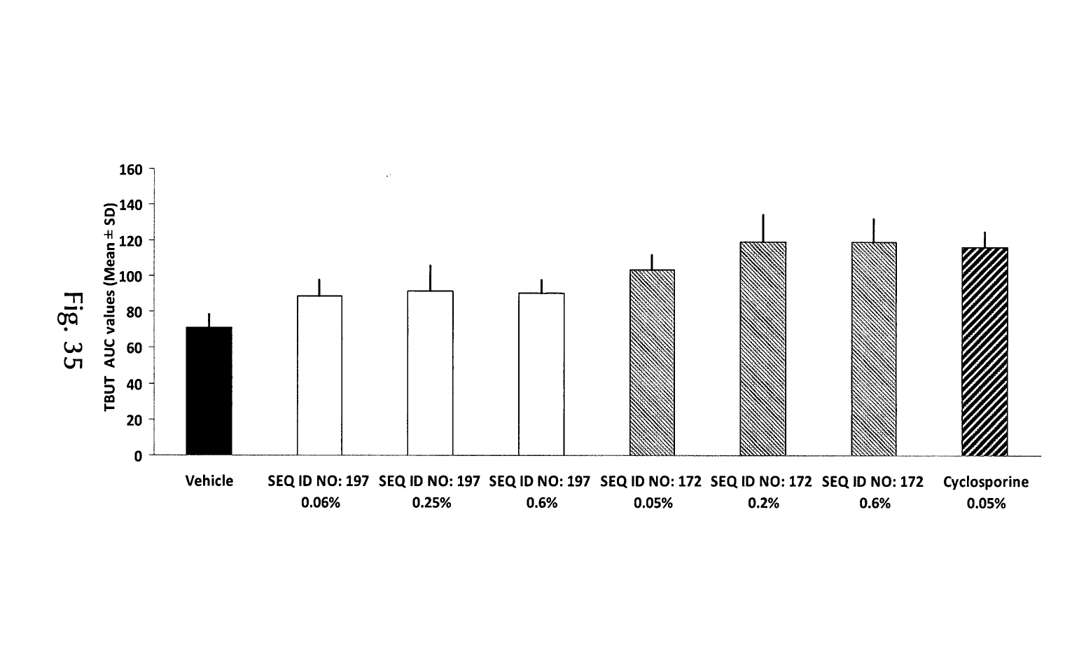

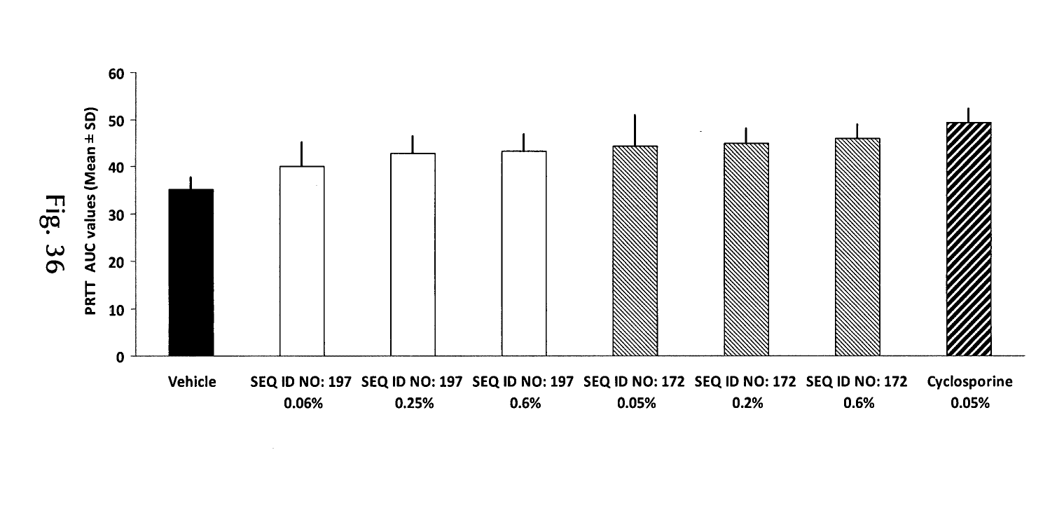

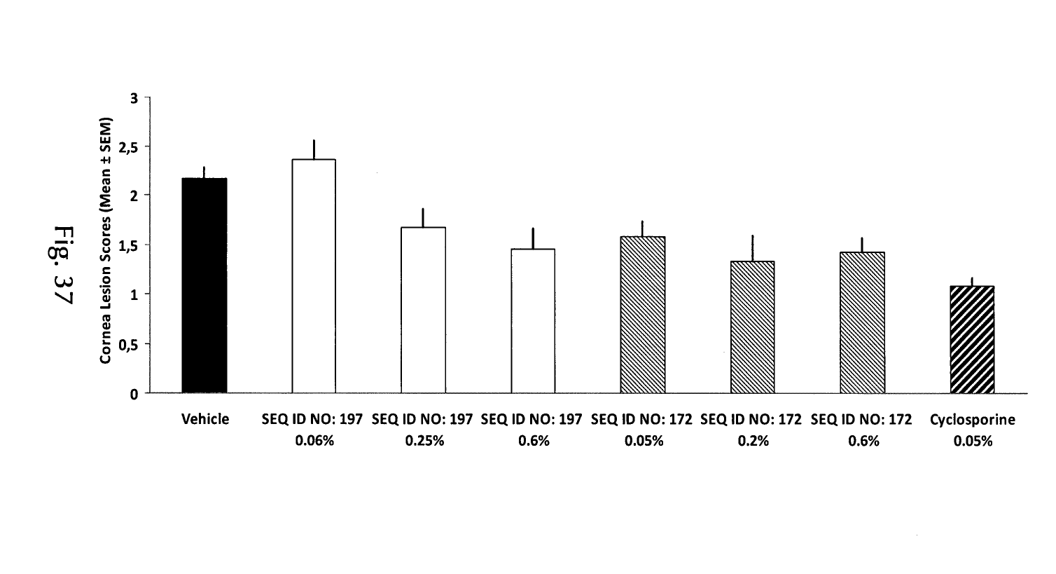

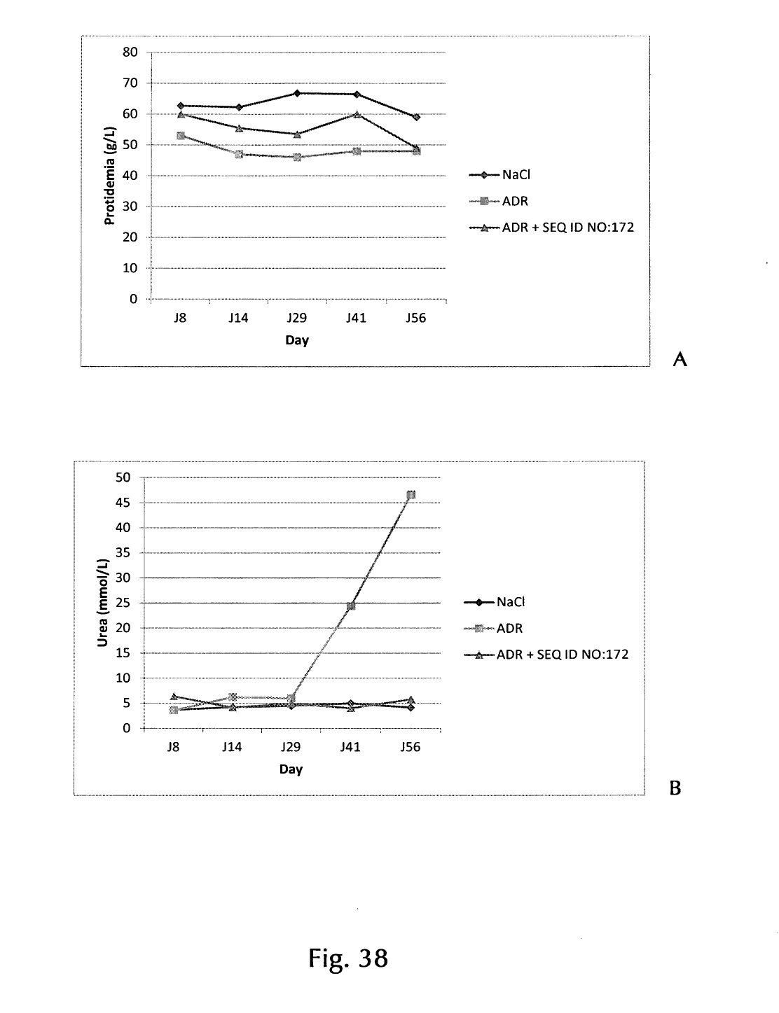

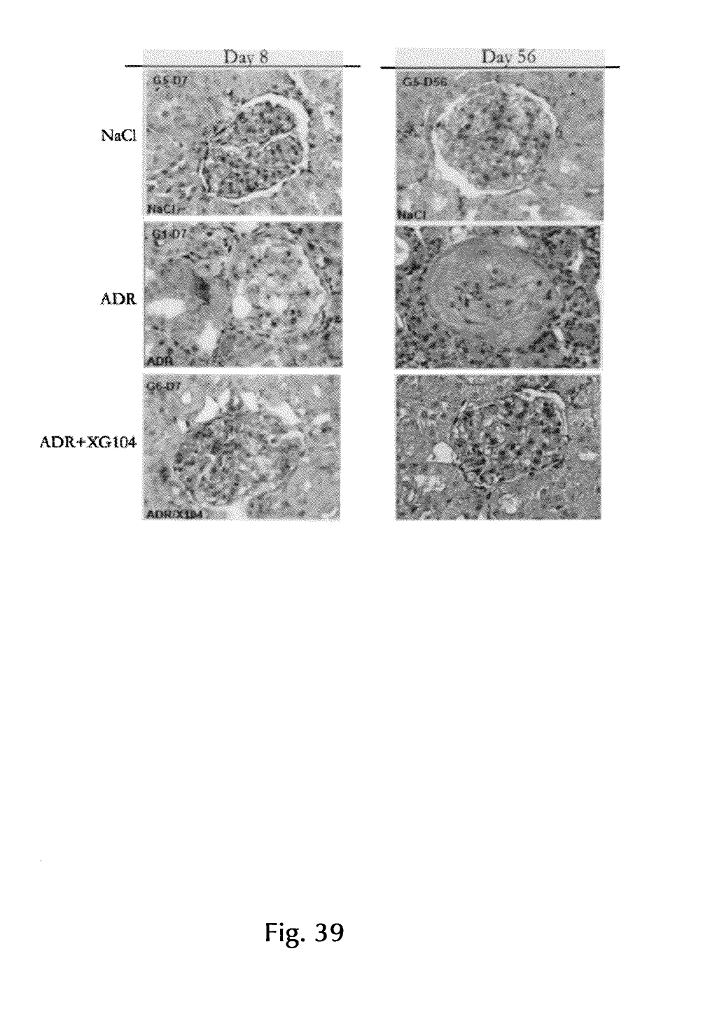

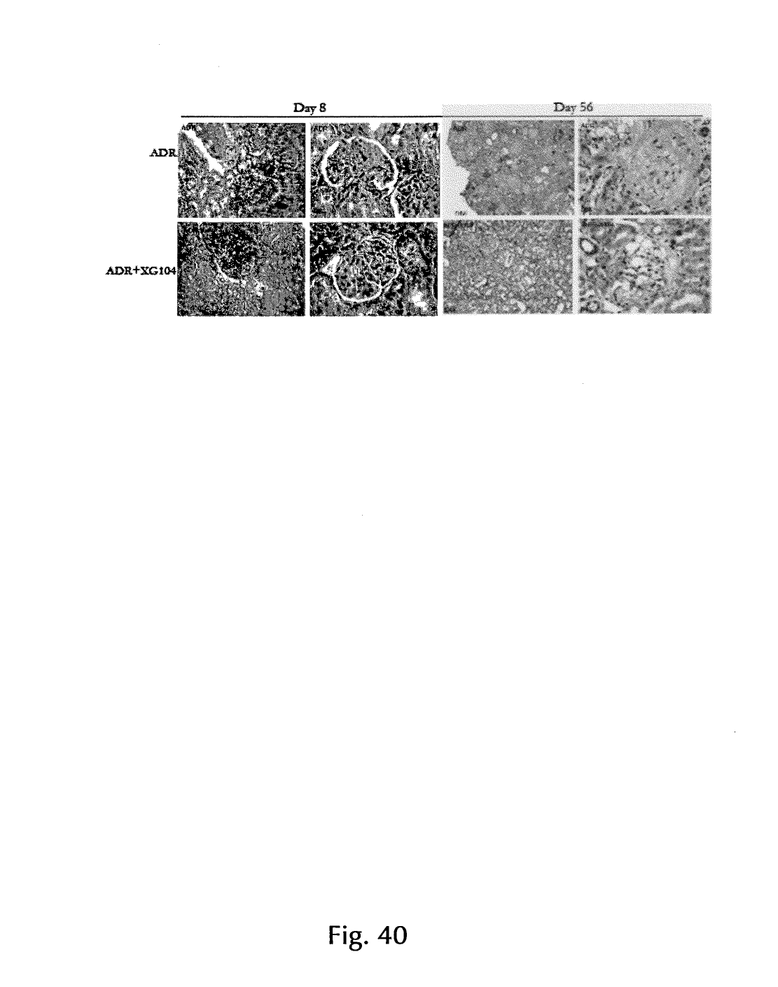

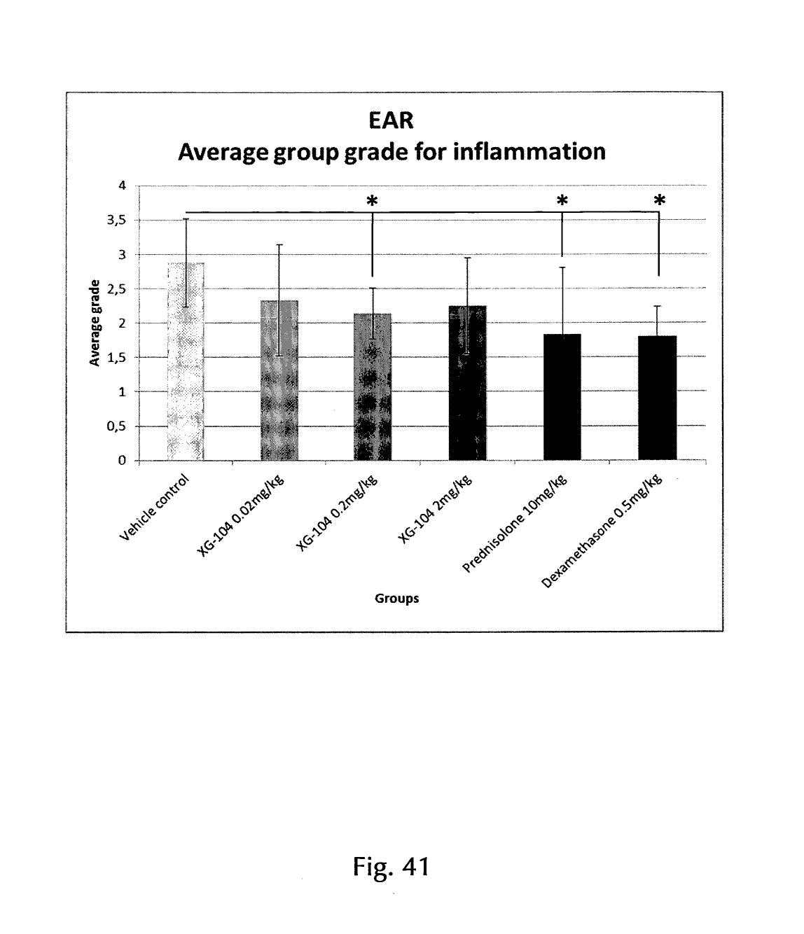

FIG. 3: Illustration of the inhibitory efficacy of several JNK inhibitors according to the present invention, which are fusion proteins of a JNK inhibitory (poly-)peptide sequence and a transporter sequence. The inhibitory efficacy was determined by means of in vitro AlphaScreen assay (Amplified Luminescence Proximity Homogeneous-Screen Assay).
FIG. 3A: Inhibition of JNK1 by SEQ ID NOs: 194, 195, 172, 200, 46, 173, 174, 175, 176, 177, 178, 179, 180, 181 and 197.
FIG. 3B: Inhibition of JNK2 by SEQ ID NOs: 194, 195, 172, 200, 46, 173, 174, 175, 176, 177, 178, 179, 180, 181 and 197.
FIG. 3C: Inhibition of JNK3 by SEQ ID NOs: 194, 195, 172, 200, 46, 173, 174, 175, 176, 177, 178, 179, 180, 181 and 197.
FIG. 3D: Inhibition of JNK1 by SEQ ID NOs: 194, 195, 172, 200, 46, 182, 183, 184, 185, 186, 187, 188, 189, 190 and 197.
FIG. 3E: Inhibition of JNK2 by SEQ ID NOs: 194, 195, 172, 200, 46, 182, 183, 184, 185, 186, 187, 188, 189, 190 and 197.
FIG. 3F: Inhibition of JNK3 by SEQ ID NOs: 194, 195, 172, 200, 46, 182, 183, 184, 185, 186, 187, 188, 189, 190 and 197.

FIG. 4: Table illustrating the inhibitory efficacy of several JNK inhibitors according to the present invention, which are fusion proteins of a JNK inhibitory (poly-)peptide sequence and a transporter sequence. Given are the IC50 values in the nM range, the respective standard error of the mean (SEM) and the number of experiments performed (n).

FIG. 5: Stability of JNK inhibitors with SEQ ID NOs: 172, 196 and 197 in 50% human serum. The JNK inhibitor with SEQ ID NO: 196 was totally degraded into amino acids residues within 6 hours (A). The JNK inhibitor with SEQ ID NO: 172 was completely degraded only after 14 days (B). The JNK inhibitor with SEQ ID NO: 197 was stable at least up to 30 days (B).

FIG. 6: shows internalization experiments using TAT derived transporter constructs with D-amino acid/L-amino acid pattern as denoted in SEQ ID NO: 30. The transporter sequences analyzed correspond to SEQ ID NOs: 52-94 plus SEQ ID NOs: 45, 47, 46, 43 and 99 (FIG. 6a) and SEQ ID NOs: 100-147 (FIG. 6b). As can be seen, all transporters with the consensus sequence rXXXrXXXr (SEQ ID NO: 31) showed a higher internalization capability than the L-TAT transporter (SEQ ID NO: 43). Hela cells were incubated 24 hours in 96 well plate with 10 mM of the respective transporters. The cells were then washed twice with an acidic buffer (0.2M Glycin, 0.15M NaCl, pH 3.0) and twice with PBS. Cells were broken by the addition of RIPA lysis buffer. The relative amount of internalized peptide was then determined by reading the fluorescence intensity (Fusion Alpha plate reader; PerkinElmer) of each extract followed by background subtraction.

FIG. 7 The JNK inhibitor with the sequence of SEQ ID NO: 172 blocks LPS-induced cytokine and chemokine release in THP1-PMA-differentiated macrophages. FIG. 7A: TNF release (THP1pma 6 h 3 ng/ml LPS); FIG. 7B: TNF-α release (THP1pma 6 h 10 ng/ml LPS); FIG. 7C: IL 6 release (THP1pma 6 h 10 ng/ml LPS); FIG. 7D: MCP1 release (THP1pma 6 h 3 ng/ml LPS).

FIG. 8 The JNK inhibitor of SEQ ID NO: 172 blocks LPS-induced IL6 release in THP1 differentiated macrophages with higher potency than D-TAT-IB1 (SEQ ID NO: 197), dTAT (SEQ ID NO: 45) and SP 600125. LPS was added for 6 h (10 ng/ml).

FIG. 9 The JNK inhibitor of SEQ ID NO: 172 blocks LPS-induced TNFα release in THP1 differentiated macrophages with higher potency than D-TAT-IB1 (SEQ ID NO: 197), dTAT (SEQ ID NO: 45) and SP 600125. LPS was added for 6 h (10 ng/ml).

FIG. 10 The JNK inhibitor of SEQ ID NO: 172 blocks LPS-induced IL-6 release in PMA differentiated macrophages with higher potency than D-TAT-IB1 (SEQ ID NO: 197) and L-TAT-IB1 (SEQ ID NO: 196). LPS was added for 6 h.

FIG. 11 The JNK inhibitor of SEQ ID NO: 172 blocks LPS-induced TNFα release in PMA differentiated macrophages with higher potency than D-TAT-IB1 (SEQ ID NO: 197) and L-TAT-IB1 (SEQ ID NO: 196).

FIG. 12 The JNK inhibitor of SEQ ID NO: 172 blocks LPS-induced TNFα release in Primary Rat Whole Blood Cells at 3 ng/ml. Given are the results for the control, 1 μM of SEQ ID NO: 172, 3 μM of SEQ ID NO: 172, and 10 μM of SEQ ID NO: 172 at different levels of LPS (ng/ml).

FIG. 13 The JNK inhibitor of SEQ ID NO: 172 blocks IL-2 secretion by primary human T-cells in response to PMA/Ionomycin.

FIG. 14 The JNK inhibitor of SEQ ID NO: 172 blocks IL-2 secretion by primary human T-cells in response to CD3/CD28 stimulation. The JNK inhibitors used are indicated by their SEQ ID NO: 172 and 197.

FIG. 15 Dose-dependent inhibition by JNK inhibitor with SEQ ID NO: 172 of CD3/CD28-induced IL-2 release in primary rat lymph-nodes purified T cells. Control rat were sacrificed and lymph-nodes were harvested. T cells further were purified (using magnetic negative selection) and plated into 96-well plates at 200.000 cells/well. Cells were treated with anti-rat CD3 and anti-rat CD28 antibodies (2 μg/mL). JNK inhibitor with SEQ ID NO: 172 was added to the cultures 1 h before CD3/CD28 treatment and IL-2 release was assessed in supernatant 24 h after treatment.

FIG. 16 Dose-dependent inhibition of CD3/CD28-induced IL-2 release in primary rat lymph nodes purified T cells: Comparison of several JNK inhibitors, namely SEQ ID NOs: 172, 197 and SP600125.

FIG. 17 Dose dependent inhibition of IL-2 release in rat whole blood stimulated with PMA+ionomycin. JNK inhibitor with SEQ ID NO: 172 was added at three different concentrations, namely 1, 3 and 10 μM 1 h before stimulation with PMA+ionomycin. Three doses of activators were added (25/500 ng/mL, 50/750 ng/mL and 50/1000 ng/mL) for 4 h. IL-2 release was assessed in supernatant. JNK inhibitor with SEQ ID NO: 172 at 10 μM did efficiently reduce PMA-iono-induced IL-2 release at the three tested activator concentrations.

FIG. 18 JNK inhibition and IL-6 release in human whole blood. The JNK inhibitor with SEQ ID NO: 172 was added at three different concentrations, namely 1, 3 and 10 μM 1 h before whole blood stimulation with LPS (0.02 ng/mL) for 4 hours. The JNK inhibitor with SEQ ID NO: 172 did reduce the LPS-induced IL-6 release in a dose-dependent manner.

FIG. 19 JNK inhibition and IL-2 release in human whole blood. The JNK inhibitor with SEQ ID NO: 172 was added at three different concentrations, namely 1, 3 and 10 μM 1 h before whole blood stimulation with PMA+ionomycin (25/700 ng/mL, 50/800 ng/mL and 50/1000 ng/mL) for 4 hours. The JNK inhibitor with SEQ ID NO: 172 did reduce the PMA+ionomycin-induced IL-2 release in a dose-dependent manner.

FIG. 20 JNK inhibition and IFN-γ release in human whole blood. The JNK inhibitor with SEQ ID NO: 172 was added at three different concentrations, namely 1, 3 and 10 μM 1 h before whole blood stimulation with PMA+ionomycin (25/700 ng/mL, 50/800 ng/mL and 50/1000 ng/mL) for 4 hours. The JNK inhibitor with SEQ ID NO: 172 did reduce the PMA+ionomycin-induced IFN-γ release in a dose-dependent manner.

FIG. 21 JNK inhibition and TNF-α release in human whole blood. The JNK inhibitor with SEQ ID NO: 172 was added at three different concentrations, namely 1, 3 and 10 μM 1 h before whole blood stimulation with PMA+ionomycin (25/700 ng/mL, 50/800 ng/ml and 50/1000 ng/mL) for 4 hours. The JNK inhibitor with SEQ ID NO: 172 did reduce the PMA+ionomycin-induced TNF-α release in a dose-dependent manner.

FIG. 22 JNK inhibition and TNF-α release in human whole blood. The JNK inhibitor with SEQ ID NO: 172 was added at three different concentrations, namely 1, 3 and 10 μM 1 h before whole blood stimulation with PHA-L (5 μg/mL) for 3 days. The JNK inhibitor with SEQ ID NO: 172 did reduce the PHA-L-induced TNF-α release in a dose-dependent manner.

FIG. 23 JNK inhibition and IL-2 release in human whole blood. The JNK inhibitor with SEQ ID NO: 172 was added at three different concentrations, namely 1, 3 and 10 μM 1 h before whole blood stimulation with PHA-L (5 μg/mL) for 3 days. The JNK inhibitor with SEQ ID NO: 172 did reduce the PHA-L-induced IL-2 release in a dose-dependent manner.

FIG. 24 JNK inhibition and TNF-α release in human whole blood. The JNK inhibitor with SEQ ID NO: 172 was added at three different concentrations, namely 1, 3 and 10 μM 1 h before whole blood stimulation with CD3+/−CD28 antibodies (2 μg/mL) for 3 days. The JNK inhibitor with SEQ ID NO: 172 did reduce the CD3/CD28-induced TNF-α release in a dose-dependent manner.

FIG. 25 Photographic illustration of in vivo anti-inflammatory properties of the JNK inhibitors with SEQ ID NO: 197 (10 μg/kg) and SEQ ID NO: 172 (10 μg/kg) after CFA (complete Freund's adjuvant) induced paw swelling. Paw swelling was induced in the left hind paw, the right hind paw was not treated.

FIG. 26 Graphical representation of in vivo anti-inflammatory properties of the JNK inhibitors with SEQ ID NO: 197 (10 μg/kg, n=4) and SEQ ID NO: 172 (10 μg/kg, n=3) after CFA (complete Freund's adjuvant) induced paw swelling. Indicated is the measured circumference of the left hind paw after treatment.

FIG. 27 Graphical representation of in vivo anti-inflammatory properties of the JNK inhibitors with SEQ ID NO: 197 (10 μg/kg) and SEQ ID NO: 172 (10 μg/kg) after CFA (complete Freund's adjuvant) induced paw swelling. Indicated is the measured in vivo cytokine release one hour after CFA induced paw swelling.

FIG. 28 Clinical evaluation of administration of different amounts of the JNK inhibitor according to SEQ ID NO: 172 in albino rats after intravenous administration (endotoxin-induced uveitis model, EIU). Form left to right: Vehicle, 0.015 mg/kg (i.v.) of SEQ ID NO: 172; 0.18 mg/kg (i.v.) of SEQ ID NO: 172; 1.8 mg/kg (i.v.) of SEQ ID NO: 172, 2 mg/kg (i.v.) of SEQ ID NO: 197 and 20 μg dexamethasone (administered directly by subconjunctival injection to the eye). Indicated is the clinical score (mean and the SEM).

FIG. 29 Responsive effects of the JNK inhibitor of SEQ ID NO: 172 after daily intravenous administration in 14 day rat chronic established Type II collagen arthritis (RTTC/SOL-1). Shown is the body weight change from day 0 to day 14. From left to right: Normal control+Vehicle (NaCl), Disease Control+Vehicle (NaCl), 5 mg/kg (i.v.) of SEQ ID NO: 172; 1 mg/kg (i.v.) of SEQ ID NO: 172; 0.1 mg/kg (i.v.) of SEQ ID NO: 172, 0.01 mg/kg (i.v.) of SEQ ID NO: 172, 0.05 mg/kg (i.v.) of dexamethasone. Indicated is the clinical score (mean and the SEM). n=4/normal group, n=8/treatment group; *p≦0.05 1-way ANOVA to disease control+Vehicle (NaCl)

FIG. 30 Responsive effects of the JNK inhibitor of SEQ ID NO: 172 after daily intravenous administration in 14 day rat chronic established Type II collagen arthritis (RTTC/SOL-1). Shown is the ankle diameter (in) over time. n=4/normal group, n=8/treatment group; *p 50.05 2-way RM ANOVA to disease control+Vehicle (NaCl).

FIG. 31 Responsive effects of the JNK inhibitor of SEQ ID NO: 172 after daily intravenous administration in 14 day rat chronic established Type II collagen arthritis (RTTC/SOL-1). Illustrated are the ankle histopathology scores regarding inflammation, pannus, cartilage damage and bone resorption. n=8 in the treatment group. *p≦0.05 Mann-Whitney U test to disease control+Vehicle (NaCl).

FIG. 32 Responsive effects of the JNK inhibitor of SEQ ID NO: 172 after daily intravenous administration in 14 day rat chronic established Type II collagen arthritis (RTTC/SOL-1). Illustrated are the knee histopathology scores regarding inflammation, pannus, cartilage damage and bone resorption. n=8 in the treatment group. *p≦0.05 Mann-Whitney U test to disease control+Vehicle (NaCl).

FIG. 33 Clinical scoring by slit lamp 24 hours after EIU induction and administration of JNK inhibitor according to SEQ ID NO: 172 (1 mg/kg i.v.) at different times prior to EIU induction. From left to right: Vehicle (0 hours); SEQ ID NO: 172 4 weeks prior to EIU induction; SEQ ID NO: 172 2 weeks prior to EIU induction; SEQ ID NO: 172 1 week prior to EIU induction; SEQ ID NO: 172 48 hours prior to EIU induction; SEQ ID NO: 172 24 hours prior to EIU induction; SEQ ID NO: 172 0 hours prior to EIU induction; Dexamethasone (2 mg/kg i.v.) 0 hours prior to EIU induction. Mean±SEM. *p<0.05 versus vehicle, **p<0.01 versus vehicle.

FIG. 34 Number of PMN cells per section quantified 24 hours after EIU induction and administration of JNK inhibitor according to SEQ ID NO: 172 (1 mg/kg i.v.) at different times prior to EIU induction. From left to right: Vehicle (0 hours); SEQ ID NO: 172 4 weeks prior to EIU induction; SEQ ID NO: 172 2 weeks prior to EIU induction; SEQ ID NO: 172 1 week prior to EIU induction; SEQ ID NO: 172 48 hours prior to EIU induction; SEQ ID NO: 172 24 hours prior to EIU induction; SEQ ID NO: 172 0 hours prior to EIU induction; Dexamethasone (2 mg/kg i.v.) 0 hours prior to EIU induction. Mean±SEM. *p<0.05 versus vehicle, **p<0.01 versus vehicle.

FIG. 35 shows the mean calculated TBUT AUC values for animals with scopolamine-induced dry eye syndrome. Shown are the results for animals treated with vehicle, 3 different concentrations of an all-D-retro-inverso JNK-inhibitor (poly-)peptide with the sequence of SEQ ID NO: 197, 3 different concentrations of a JNK-inhibitor (poly-)peptide with the sequence of SEQ ID NO: 172, and the results for animals treated with cyclosporine.

FIG. 36 shows the mean calculated PRTT AUCs for animals with scopolamine induced Dry Eye (Day 7-21). Shown are the results for animals treated with vehicle, 3 different concentrations of an all-D-retro-inverso JNK-inhibitor (poly-)peptide with the sequence of SEQ ID NO: 197, 3 different concentrations of a JNK-inhibitor (poly-)peptide with the sequence of SEQ ID NO: 172, and the results for animals treated with cyclosporine.

FIG. 37 shows the mean histological Cornea Lesion Scores for animals with scopolamine induced dry eye syndrome. Shown are the results for animals treated with vehicle, 3 different concentrations of an all-D-retro-inverso JNK-inhibitor (poly-)peptide with the sequence of SEQ ID NO: 197, 3 different concentrations of a JNK-inhibitor (poly-)peptide with the sequence of SEQ ID NO: 172, and the results for animals treated with cyclosporine.

FIG. 38 shows the renal function assessed by protidemia (A) and urea level (B) of rats in an Adriamycin (ADR)-induced nephropathy model on Days 8, 14, 29, 41 and 56 after ADR administration. Groups No. 1 (“ADR”) and No. 2 (“ADR+JNK inhibitor SEQ Id NO: 172”) have been treated on Day 0 with ADR to induce necropathy, whereas group No. 3 (“NaCl”) received 0.9% NaCL. Moreover, group No. 2 (“ADR+JNK inhibitor SEQ Id NO: 172”) has been treated on Day 0 with the JNK inhibitor SEQ Id NO: 172, whereas groups No. 1 and 3 received vehicle (0.9% NaCl).

FIG. 39 shows kidney sections of the rats in the Adriamycin (ADR)-induced nephropathy model stained with periodic acid-Schiff (PAS) (original magnification ×40). For the sections shown in the left column, rats were sacrificed at Day 8 following ADR administration, whereas for the sections shown in the left column, rats were sacrificed at Day 56. ADR has been administered only to the groups “ADR” and “ADR+XG104”, whereas the group “NaCl” received 0.9% NaCL only. The group “ADR+XG104” has been treated on Day 0 with the JNK inhibitor SEQ Id NO: 172 (i.e. “XG104” refers to the JNK inhibitor SEQ Id NO: 172), whereas the other groups (“ADR” and “NaCl”) received vehicle (0.9% NaCl).

FIG. 40 shows the kidney fibrosis in ADR nephropathy evaluated with Masson's trichrome (blue) on Days 8 (left four panels) and 56 (right four panels) following ADR administration for the group “ADR” (upper panel), which has been treated with ADR and vehicle at Day 0 and for the group “ADR+XG104” (lower panel), which has been treated with ADR and the JNK inhibitor SEQ Id NO: 172 at Day 0. The original magnification ×10 is depicted in the left panels for the respective day and the original magnification ×40 is depicted in the right panels for the respective day.

FIG. 41 shows the average group grade for inflammation of the ear in an iquimod-induced psoriasis-model in mice after six consecutive days of iquimod application. The “average grade” refers to the microscopic histopathology end-points (cf. Example 14). Three doses (0.02, 0.2 and 2 mg/kg) of the JNK inhibitor of SEQ Id NO: 172 have been tested (groups “XG-104 0.02 mg/kg, XG-104 0.2 mg/kg, and XG-104 2 mg/kg, respectively). Prednisolone and dexamethasone served as positive controls. The groups XG-104 0.2 mg/kg, prednisolone and dexamethasone showed significant differences from the vehicle control group.

### JNK INHIBITORS

In a first aspect the present invention relates to a JNK inhibitor, which comprises an inhibitory (poly-)peptide sequence according to the following general formula:

(SEQ ID NO: 1)
X1-X2-X3-R-X4-X5-X6-L-X7-L-X8,

wherein X1 is an amino acid selected from amino acids R, P, Q and r,
wherein X2 is an amino acid selected from amino acids R, P, G and r,
wherein X3 is an amino acid selected from amino acids K, R, k and r,
wherein X4 is an amino acid selected from amino acids P and K,
wherein X5 is an amino acid selected from amino acids T, a, s, q, k or is absent,
wherein X6 is an amino acid selected from amino acids T, D and A,
wherein X7 is an amino acid selected from amino acids N, n, r and K; and
wherein X8 is an amino acid selected from F, f and w,
with the proviso that at least one, at least two, at least three, at least four, at least five or six of the amino acids selected from the group consisting of X1, X2, X3, X5, X7 and X8 is/are a D-amino acid(s), preferably with the proviso that at least one, at least two, at least three or four of the amino acids selected from the group consisting of X3, X5, X7 and X8 is/are a D-amino acid(s),
   for use in a method for treatment of the human or animal body by therapy, in particular for the treatment of the diseases/disorders disclosed herein.

The inhibitory (poly-)peptide sequence of the JNK inhibitor according to the present invention comprises L-amino acids and in most embodiments D-amino acids. Unless specified otherwise, L-amino acid residues are indicated herein in capital letters, while D amino acid residues are indicated in small letters. Glycine may be indicated in capital or small letters (since there is no D- or L-glycine). The amino acid sequences disclosed herein are always given from N- to C-terminus (left to right) unless specified otherwise. The given amino acid sequence may be modified or unmodified at the C- and/or N-terminus, e.g. acetylation at the C-terminus and/or amidation or modification with cysteamide at the N-terminus. Such conceivable, but optional modifications at the C- and/or N-terminus of the amino acid sequences disclosed herein are—for sake of clarity—not specifically indicated.

The JNK inhibitors of the present invention are (poly-)peptide inhibitors of the c-Jun N-terminal kinase (JNK). Said inhibitors inhibit the kinase activity of c-Jun N-terminal kinase (JNK), i.e. prevent or reduce the extent of phosphorylation of JNK substrates, such as c-Jun, ATF2 and/or Elk-1 by e.g. blocking the JNK activity. A person skilled in the art will understand that the term “inhibitor”, as used herein, does not comprise compounds which irreversibly destroy the c-Jun N-terminal kinase (JNK) molecule and/or kinase activity. Accordingly, the JNK inhibitory activity of the inhibitors of the present invention typically refers to compounds which bind in a competitive or non-competitive manner to JNK. Furthermore, the term “inhibiting JNK activity” as used herein, refers to the inhibition of the kinase activity of c-Jun N-terminal kinase (JNK).

Furthermore, as used herein, a JNK inhibitor comprises at least one functional unit of a polymer of amino acids, i.e. a (poly-)peptide sequence. Moreover, this at least one functional polymer of amino acids provides for inhibition of JNK activity. The amino acid monomers of said inhibitory (poly-)peptide sequence are usually linked to each other via peptide bonds, but (chemical) modifications of said peptide bond(s) or of side chain residues may be tolerable, provided the inhibitory activity (inhibition of JNK activity) is not totally lost, i.e. the resulting chemical entity still qualifies as JNK inhibitor as functionally defined herein. The term “(poly-)peptide” shall not be construed as limiting the length of the (poly-)peptide unit. Preferably, the inhibitory (poly-)peptide sequence of the JNK inhibitors of the present invention is less than 500, 490, 480, 470, 460, 450, 440, 430, 420, 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 95, 90, 85, 80, 75, 70, 65, 60, 55, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, or less than 12 amino acids long. Preferably, the inhibitory (poly-)peptide sequence does not have less than 10 amino acid residues, more preferably not less than 11 amino acid residues.

Furthermore, a “JNK inhibitor” of the present invention inhibits JNK activity, e.g. exhibits with regard to the inhibition of human JNK mediated phosphorylation of a c-Jun substrate (SEQ ID NO: 198) an IC 50 value of:
a) less than 3000 nM, more preferably less than 2000 nM, even more preferably less than 1000 nM, even more preferably less than 500 nM, even more preferably less than 250 nM, even more preferably less than 200 nM, even more preferably less than 150 nM, most preferably less than 100 nM with regard to inhibition of human JNK1,
b) less than 3000 nM, more preferably less than 2000 nM, even more preferably less than 1000 nM, even more preferably less than 500 nM, even more preferably less than 250 nM, even more preferably less than 200 nM, even more preferably less than 150 nM, most preferably less than 100 nM with regard to inhibition of human JNK2, and/or
c) less than 3000 nM, more preferably less than 2000 nM, even more preferably less than 1000 nM, even more preferably less than 500 nM, even more preferably less than 250 nM, even more preferably less than 200 nM, even more preferably less than 150 nM, most preferably less than 100 nM with regard to inhibition of human JNK3.

For some applications, it is preferred that the inhibitor inhibits human JNK2 and/or human JNK3 according to the above definition, but not JNK1 according to the above definition.

Whether JNK activity is inhibited or not, may easily be assessed by a person skilled in the art. There are several methods known in the art. One example is a radioactive kinase assay or a non-radioactive kinase assay (e.g. Alpha screen test; see for example Guenat et al. J Biomol Screen, 2006; 11: pages 1015-1026).

A JNK inhibitor according to the present invention may thus for example comprise an inhibitory (poly-)peptide sequence according to any of SEQ ID NOs: 2 to 27 (see table 1).

TABLE 1
Examples for inhibitory (poly-)peptide sequences
of JNK-inhibitors according to the present
invention

| Amino acid sequence | SEQ ID NO: |
|---|---|
| rPKRPTTLNLF | 2 |
| RPkRPTTLNLF | 3 |
| RPKRPaTLNLF | 4 |
| RPKRPTTLnLF | 5 |
| RPKRPTTLrLF | 6 |
| RPKRPTTLNLf | 7 |
| RPkRPaTLNLf | 8 |
| RPkRPTTLNLf | 9 |
| RPkRPTTLrLf | 10 |
| RRrRPTTLNLf | 11 |
| QRrRPTTLNLf | 12 |
| RPkRPTTLNLw | 13 |
| RPkRPTDLNLf | 14 |
| RRrRPTTLrLw | 15 |
| QRrRPTTLrLw | 16 |
| RRrRPTDLrLw | 17 |
| QRrRPTDLrLw | 18 |
| RRrRPaTLNLf | 19 |
| QRrRPaTLNLf | 20 |
| RrKRPaTLNLf | 21 |
| RPkRPsTLNLf | 22 |
| RPkRPqTLNLf | 23 |
| RPkRPkTLNLf | 24 |
| rGKRKALKLf | 25 |
| rGKRKALrLf | 26 |
| RRrRKALrLf | 27 |

The JNK inhibitor according to the present invention may also be a JNK inhibitor (variant) which comprises an inhibitory (poly-)peptide sequence sharing at least 50%, more preferably at least 55%, more preferably at least 60%, more preferably at least 65%, more preferably at least 70%, more preferably at least 75%, more preferably at least 80%, more preferably at least 85%, most preferably at least 90%, more preferably at least 95% sequence identity with a sequence selected from SEQ ID NOs: 1-27, in particular with SEQ ID NO: 8,

with the proviso that with regard to the respective sequence selected from SEQ ID NOs: 1-27, such inhibitory (poly-)peptide sequence sharing sequence identity
a) maintains the L-arginine (R) residue on position 4,
b) maintains the two L-leucine (L) residues at position 8 and 10 (positions 7 and 9 with regard to SEQ ID NOs: 25-27),
c) exhibits one, two, three, four, five or six D-amino acid(s) at the respective positions corresponding to the amino acids selected from the group consisting of X1, X2, X3, X5, X7 and X8 of SEQ ID NO: 1 and respective positions in SEQ ID NOs: 2-27, more preferably exhibits one, two, three or four D-amino acid(s) at the positions corresponding to the amino acids selected from the group consisting of X3, X5, X7 and X8 of SEQ ID NO: 1 and respective positions in SEQ ID NOs: 2-27, and
d) still inhibits JNK activity (i.e. is a JNK inhibitor as defined herein).

Certainly, variants disclosed herein (in particular JNK inhibitor variants comprising an inhibitory (poly-)peptide sequence sharing—within the above definition—a certain degree of sequence identity with a sequence selected from SEQ ID NOs: 1-27), share preferably less than 100% sequence identity with the respective reference sequence.

In view of said definition and for sake of clarity the residues which may preferably not be altered variants of JNK inhibitors comprising SEQ ID NOs: 1-27 (see a) and b) in the above definition) are underlined in table 1.

The non-identical amino acids are preferably the result of conservative amino acid substitutions.

Conservative amino acid substitutions, as used herein, may include amino acid residues within a group which have sufficiently similar physicochemical properties, so that a substitution between members of the group will preserve the biological activity of the molecule (see e.g. Grantham, R. (1974), Science 185, 862-864). Particularly, conservative amino acid substitutions are preferably substitutions in which the amino acids originate from the same class of amino acids (e.g. basic amino acids, acidic amino acids, polar amino acids, amino acids with aliphatic side chains, amino acids with positively or negatively charged side chains, amino acids with aromatic groups in the side chains, amino acids the side chains of which can enter into hydrogen bridges, e.g. side chains which have a hydroxyl function, etc.). Conservative substitutions are in the present case for example substituting a basic amino acid residue (Lys, Arg, His) for another basic amino acid residue (Lys, Arg, His), substituting an aliphatic amino acid residue (Gly, Ala, Val, Leu, Ile) for another aliphatic amino acid residue, substituting an aromatic amino acid residue (Phe, Tyr, Trp) for another aromatic amino acid residue, substituting threonine by serine or leucine by isoleucine. Further conservative amino acid exchanges will be known to the person skilled in the art. The isomer form should preferably be maintained, e.g. K is preferably substituted for R or H, while k is preferably substituted for r and h.

Further possible substitutions within the above definition for JNK inhibitor variants are for example:
a) one, two or more of X1, X2, X3, X4, X5, X6, X7 and/or X8 of SEQ ID NO: 1 or the corresponding positions within the respective sequence selected from SEQ ID NOs: 2-27 are substituted for A or a,
b) X1 or X8 of SEQ ID NO: 1 or the corresponding position within the respective sequence selected from SEQ ID NOs: 2-27 is deleted;
c) X5 of SEQ ID NO: 1 or the corresponding position within the respective sequence selected from SEQ ID NOs: 2-27 is E, Y, L, V, F or K;
d) X5 of SEQ ID NO: 1 or the corresponding position within the respective sequence selected from SEQ ID NOs: 2-27 is E, L, V, F or K; or
e) one, two or three of X1, X2, X3 of SEQ ID NO: 1 or the corresponding positions within the respective sequence selected from SEQ ID NOs: 2-27 are neutral amino acids.

As used herein, the term “% sequence identity”, has to be understood as follows: Two sequences to be compared are aligned to give a maximum correlation between the sequences. This may include inserting “gaps” in either one or both sequences, to enhance the degree of alignment. A % identity may then be determined over the whole length of each of the sequences being compared (so-called global alignment), that is particularly suitable for sequences of the same or similar length, or over shorter, defined lengths (so-called local alignment), that is more suitable for sequences of unequal length. In the above context, an amino acid sequence having a “sequence identity” of at least, for example, 95% to a query amino acid sequence, is intended to mean that the sequence of the subject amino acid sequence is identical to the query sequence except that the subject amino acid sequence may include up to five amino acid alterations per each 100 amino acids of the query amino acid sequence. In other words, to obtain an amino acid sequence having a sequence of at least 95% identity to a query amino acid sequence, up to 5% (5 of 100) of the amino acid residues in the subject sequence may be inserted or substituted with another amino acid or deleted. For purposes of determining sequence identity, the substitution of an L-amino acid for a D-amino acid (and vice versa) is considered to yield a non-identical residue, even if it is merely the D- (or L-isomer) of the very same amino acid.

Methods for comparing the identity and homology of two or more sequences are well known in the art. The percentage to which two sequences are identical can for example be determined by using a mathematical algorithm. A preferred, but not limiting, example of a mathematical algorithm which can be used is the algorithm of Karlin et al. (1993), PNAS USA, 90:5873-5877. Such an algorithm is integrated in the BLAST family of programs, e.g. BLAST or NBLAST program (see also Altschul et al., 1990, J. Mol. Biol. 215, 403-410 or Altschul et al. (1997), Nucleic Acids Res, 25:3389-3402), accessible through the home page of the NCBI at world wide web site ncbi.nlm.nih.gov) and FASTA (Pearson (1990), Methods Enzymol. 183, 63-98; Pearson and Lipman (1988), Proc. Natl. Acad. Sci. U.S.A 85, 2444-2448.). Sequences which are identical to other sequences to a certain extent can be identified by these programmes. Furthermore, programs available in the Wisconsin Sequence Analysis Package, version 9.1 (Devereux et al, 1984, Nucleic Acids Res., 387-395), for example the programs BESTFIT and GAP, may be used to determine the % identity between two polypeptide sequences. BESTFIT uses the “local homology” algorithm of (Smith and Waterman (1981), J. Mol. Biol. 147, 195-197.) and finds the best single region of similarity between two sequences.

Certainly, the JNK inhibitor according to the present invention may comprise—in addition to the inhibitory (poly-)peptide sequence mentioned above—additional sequences or sequence elements, domains, labels (e.g. fluorescent or radioactive labels), epitopes etc., as long as the ability to inhibit JNK activity as defined herein is not lost. For example, the JNK inhibitor according to the present invention may also comprise a transporter sequence. A “transporter sequence” as used herein, is a (poly-)peptide sequence providing for translocation of the molecule it is attached to across biological membranes. Accordingly, a JNK inhibitor according to the present invention comprising a transporter sequence is preferably capable of translocating (e.g. the conjugated cargo compound) across biological membranes. Thus, such JNK inhibitors of the present invention may more readily enter into a cell, a cellular subcompartment and/or into the nucleus of a cell.

Said transporter sequence may be joined for example (e.g. directly) N-terminally or (e.g. directly) C-terminally to the inhibitory (poly-)peptide sequence of the JNK inhibitor, preferably by a covalent linkage. The transporter sequence and the inhibitory (poly-)peptide sequence may also be spaced apart, e.g. may be separated by intermediate or linker sequences. It is also contemplated that the transporter sequence may be positioned entirely elsewhere in the JNK inhibitor molecule than the inhibitory (poly-)peptide sequence, in particular if the JNK inhibitor is a more complex molecule (e.g. comprising several domains, is a multimeric conjugate etc.). It is also contemplated that the transporter sequence and the inhibitory (poly-)peptide sequence may overlap. However, the JNK inhibitory activity of the JNK inhibitory portion needs to be maintained. Examples for such overlapping instances are given further below.

Transporter sequences for use with the JNK inhibitor of the present invention may be selected from, without being limited thereto, transporter sequences derived from HIV TAT (HIV), e.g. native proteins such as e.g. the TAT protein (e.g. as described in U.S. Pat. Nos. 5,804,604 and 5,674,980, each of these references being incorporated herein by reference), HSV VP22 (Herpes simplex) (described in e.g. WO 97/05265; Elliott and O'Hare, Cell 88: 223-233 (1997)), non-viral proteins (Jackson et al, Proc. Natl. Acad. Sci. USA 89: 10691-10695 (1992)), transporter sequences derived from Antennapedia, particularly from Drosophila antennapedia (e.g. the antennapedia carrier sequence thereof), FGF, lactoferrin, etc. or derived from basic peptides, e.g. peptides having a length of at least 5 or at least 10 or at least 15 amino acids, e.g. 5 to 15 amino acids, preferably 10 to 12 amino acids, Such transporter sequences preferably comprise at least 50%, more preferably at least 80%, more preferably 85% or even 90% basic amino acids, such as e.g. arginine, lysine and/or histidine, or may be selected from e.g. arginine rich peptide sequences, such as RRRRRRRRR (R₉; SEQ ID NO: 152), RRRRRRRR (R₈; SEQ ID NO: 153), RRRRRRR (R₇; SEQ ID NO: 154), RRRRRR (R₆, SEQ ID NO: 155), RRRRR (R₅, SEQ ID NO: 156) etc., from VP22, from PTD-4 proteins or peptides, from RGD-K16, from PEPT1/2 or PEPT1/2 proteins or peptides, from SynB3 or SynB3 proteins or peptides, from PC inhibitors, from P21 derived proteins or peptides, or from JNKI proteins or peptides.

Examples of transporter sequences for use in the JNK inhibitor of the present invention are in particular, without being limited thereto, basic transporter sequences derived from the HIV-1 TAT protein. Preferably, the basic transporter sequence of the HIV-1 TAT protein may include sequences from the human immunodeficiency virus HIV-1 TAT protein, e.g. as described in, e.g., U.S. Pat. Nos. 5,804,604 and 5,674,980, each incorporated herein by reference. In this context, the full-length HIV-1 TAT protein has 86 amino acid residues encoded by two exons of the HIV TAT gene. TAT amino acids 1-72 are encoded by exon 1, whereas amino acids 73-86 are encoded by exon 2. The full-length TAT protein is characterized by a basic region which contains two lysines and six arginines (amino acids 49-57) and a cysteine-rich region which contains seven cysteine residues (amino acids 22-37). The basic region (i.e., amino acids 49-57) was thought to be important for nuclear localization. Ruben, S. et al., J. Virol. 63: 1-8 (1989); Hauber, J. et al., J. Virol. 63 1181-1187 (1989). The cysteine-rich region mediates the formation of metal-linked dimers in vitro (Frankel, A. D. et al, Science 240: 70-73 (1988); Frankel, A. D. et al., Proc. Natl. Acad. Sci USA 85: 6297-6300 (1988)) and is essential for its activity as a transactivator (Garcia, J. A. et al., EMBO J. 7: 3143 (1988); Sadaie, M. R. et al., J. Virol. 63:1 (1989)). As in other regulatory proteins, the N-terminal region may be involved in protection against intracellular proteases (Bachmair, A. et al, Cell 56: 1019-1032 (1989)). Preferred TAT transporter sequences for use in the JNK inhibitor of the present invention are preferably characterized by the presence of the TAT basic region amino acid sequence (amino acids 49-57 of naturally-occurring TAT protein); the absence of the TAT cysteine-rich region amino acid sequence (amino acids 22-36 of naturally-occurring TAT protein) and the absence of the TAT exon 2-encoded carboxy-terminal domain (amino acids 73-86 of naturally-occurring TAT protein). More preferably, the transporter sequence in the JNK inhibitor of the present invention may be selected from an amino acid sequence containing TAT residues 48-57 or 49 to 57 or variants thereof.

Preferably, the transporter sequence in a given JNK inhibitor of the present invention also exhibits D-amino acids, for example in order to improve stability towards proteases. Particularly preferred are transporter sequences which exhibit a specific order of alternating D- and L-amino acids. Such order of alternating D- and L-amino acids (the motif) may follow—without being limited thereto—the pattern of any one of SEQ ID NOs: 28-30:

(SEQ ID NO: 28)
dₗLLLₓdₘLLLydₙ;
(SEQ ID NO: 29)
dLLLd(LLLd)ₐ;
and/or
(SEQ ID NO: 30)
dLLLdLLLd;

wherein: d is a D-amino acid;
L is a L-amino acid;
a is 0-3, preferably 0-2, more preferably 0, 1, 2 or 3, even more preferably 0, 1, or 2 and most preferably 1;
l, m and n are independently from each other 1 or 2, preferably 1;
x and y are independently from each other 0, 1 or 2, preferably 1.

Said order of D- and L-amino acids (motif) becomes relevant when the transporter sequence is synthesized, i.e. while the amino acid sequence (i.e. the type of side chain residues) remains unaltered, the respective isomers alternate. For example, a known transporter sequence derived from HIV TAT is RKKRRQRRR (SEQ ID NO: 43). Applying the D-/L amino acid order of SEQ ID NO: 30 thereto would yield rKKRrQRRr (SEQ ID NO: 46).

In a particular embodiment the transporter sequence of the JNK inhibitor of the present invention may comprise at least one sequence according to rXXXrXXXr (SEQ ID NO: 31), wherein:
r represents an D-enantiomeric arginine;
X is any L-amino acid (including glycine);
   and wherein each X may be selected individually and independently of any other X within SEQ ID NO: 31. Preferably at least 4 out of said 6 X L-amino acids within SEQ ID NO: 31 are K or R. In another embodiment the JNK inhibitor according to the present invention comprises the transporter sequence rX₁X₂X₃rX₄X₅X₆r (SEQ ID NO: 32), wherein X₁ is K, X₂ is K, X₃ is R and X₄, X₅, and X₆ are any L-amino acid (including glycine) selected independently from each other. Similarly, the transporter sequence of the JNK inhibitor according to the present invention may comprise the sequence rX₁X₂X₃rX₄X₅X₆r (SEQ ID NO: 33), wherein X₄ is Q, X₅ is R, X₆ is R and X₁, X₂, and X₃ are any L-amino acid (including glycine) selected independently from each other. The inventive JNK inhibitor may also comprise the sequence rX₁X₂X₃rX₄X₅X₆r (SEQ ID NO: 34), wherein one, two, three, four, five or six X amino acid residues are chosen from the group consisting of: X₁, is K, X₂ is K, X₃ is R, X₄ is Q, X₅ is R, X₆ is R, while the remaining X amino acid residues not selected from above group may be any L-amino acid (including glycine) and are selected independently from each other. X₁ is then preferably Y and/or X₄ is preferably K or R.

Examples of transporter sequences for use in the inventive JNK inhibitor molecule may be selected, without being limited thereto, from sequences as given in table 2 below, (SEQ ID NOs: 31-170) or from any fragment or variant or chemically modified derivative thereof (preferably it retains the function of translocating across a biological membrane).

TABLE 2
Examples for transporter (poly-)peptide sequences for use in the JNK-inhibitors
according to the present invention

| SEQUENCE/PEPTIDE | SEQ ID | | |
|---|---|---|---|
| NAME | NO | AA | SEQUENCE |
| r3 (generic) | 31 | 9 | rXXXrXXXr |
| r3 (generic; right half) | 32 | 9 | rKKRrX₄X₅X₆r |
| r3 (generic; left half) | 33 | 9 | rX₁X₂X₃rQRRr |
| r3 (generic; individual) | 34 | 9 | rX₁X₂X₃rX₄X₅X₆r |
| TAT (1-86) | 35 | 86 | MEPVDPRLEP WKHPGSQPKT ACTNCYCKKC CFHCQVCFIT |
| | | | KALGISYGRK KRRQRRRPPQ GSQTHQVSLS KQPTSQSRGD |
| | | | PTGPKE |
| TAT (37-72) | 36 | 36 | CFITKALGIS YGRKKRRQRR RPPQGSQTHQ VSLSKQ |
| TAT (37-58) | 37 | 22 | CFITKALGIS YGRKKRRQRR RP |
| TAT (38-58)GGC | 38 | 24 | FITKALGISY GRKKRRQRRR PGGC |
| TAT CGG(47-58) | 39 | 15 | CGGYGRKKRR QRRRP |
| TAT (47-58)GGC | 40 | 15 | YGRKKRRQRR RPGGC |
| TAT (1-72) Mut | 41 | 56 | MEPVDPRLEP WKHPGSQPKT AFITKALGIS YGRKKRRQRR |
| Cys/Ala 72 | | | RPPQGSQTHQ VSLSKQ |
| L-TAT (s1a) | 42 | 10 | GRKKRRQRRR |
| | | | (NH₂-GRKKRRQRRR-COOH) |
| L-TAT (s1b) | 43 | 9 | RKKRRQRRR |
| | | | (NH₂-GRKKRRQRRR-COOH) |
| L-TAT (s1c) | 44 | 11 | YDRKKRRQRRR |
| D-TAT | 45 | 9 | rrrqrrkkr |
| r₃-L-TAT | 46 | 9 | rKKRrQRRr |
| r₃-L-TATi | 47 | 9 | rRRQrRKKr |
| βA-r₃-L-TAT | 48 | 9 | βA-rKKRrQRRr (βA: beta alanine) |
| βA-r₃-L-TATi | 49 | 9 | βA-rRRQrRKKr (βA: beta alanine) |
| FITC-βA-r₃-L-TAT | 50 | 9 | FITC-βA-rKKRrQRRr (βA: beta alanine) |
| FITC-βA-r₃-L-TATi | 51 | 9 | FITC-βA-rRRQrRKKr (βA: beta alanine) |
| TAT(s2-1) | 52 | 9 | rAKRrQRRr |
| TAT(s2-2) | 53 | 9 | rKARrQRRr |
| TAT(s2-3) | 54 | 9 | rKKArQRRr |
| TAT(s2-4) | 55 | 9 | rKKRrARRr |
| TAT(s2-5) | 56 | 9 | rKKRrQARr |
| TAT(s2-6) | 57 | 9 | rKKRrQRAr |
| TAT(s2-7) | 58 | 9 | rDKRrQRRr |
| TAT(s2-8) | 59 | 9 | rKDRrQRRr |
| TAT(s2-9) | 60 | 9 | rKKDrQRRr |
| TAT(s2-10) | 61 | 9 | rKKRrDRRr |
| TAT(s2-11) | 62 | 9 | rKKRrQDRr |
| TAT(s2-12) | 63 | 9 | rKKRrQRDr |
| TAT(s2-13) | 64 | 9 | rEKRrQRRr |
| TAT(s2-14) | 65 | 9 | rKERrQRRr |
| TAT(s2-15) | 66 | 9 | rKKErQRRr |
| TAT(s2-16) | 67 | 9 | rKKRrERRr |
| TAT(s2-17) | 68 | 9 | rKKRrQERr |
| TAT(s2-18) | 69 | 9 | rKKRrQREr |
| TAT(s2-19) | 70 | 9 | rFKRrQRRr |
| TAT(s2-20) | 71 | 9 | rKFRrQRRr |
| TAT(s2-21) | 72 | 9 | rKKFrQRRr |
| TAT(s2-22) | 73 | 9 | rKKRrFRRr |
| TAT(s2-23) | 74 | 9 | rKKRrQFRr |
| TAT(s2-24) | 75 | 9 | rKKRrQRFr |
| TAT(s2-25) | 76 | 9 | rRKRrQRRr |
| TAT(s2-26) | 77 | 9 | rKRRrQRRr |
| TAT(s2-27) | 78 | 9 | rKKKrQRRr |
| TAT(s2-28) | 79 | 9 | rKKRrRRRr |
| TAT(s2-29) | 80 | 9 | rKKRrQKRr |
| TAT(s2-30) | 81 | 9 | rKKRrQRKr |
| TAT(s2-31) | 82 | 9 | rHKRrQRRr |
| TAT(s2-32) | 83 | 9 | rKHRrQRRr |
| TAT(s2-33) | 84 | 9 | rKKHrQRRr |
| TAT(s2-34) | 85 | 9 | rKKRrHRRr |
| TAT(s2-35) | 86 | 9 | rKKRrQHRr |
| TAT(s2-36) | 87 | 9 | rKKRrQRHr |
| TAT(s2-37) | 88 | 9 | rIKRrQRRr |
| TAT(s2-38) | 89 | 9 | rKIRrQRRr |
| TAT(s2-39) | 90 | 9 | rKKIrQRRr |
| TAT(s2-40) | 91 | 9 | rKKRrIRRr |
| TAT(s2-41) | 92 | 9 | rKKRrQIRr |
| TAT(s2-42) | 93 | 9 | rKKRrQRIr |
| TAT(s2-43) | 94 | 9 | rLKRrQRRr |
| TAT(s2-44) | 95 | 9 | rKLRrQRRr |
| TAT(s2-45) | 96 | 9 | rKKLrQRRr |
| TAT(s2-46) | 97 | 9 | rKKRrLRRr |
| TAT(s2-47) | 98 | 9 | rKKRrQLRr |
| TAT(s2-48) | 99 | 9 | rKKRrQRLr |
| TAT(s2-49) | 100 | 9 | rMKRrQRRr |
| TAT(s2-50) | 101 | 9 | rKMRrQRRr |
| TAT(s2-51) | 102 | 9 | rKKMrQRRr |
| TAT(s2-52) | 103 | 9 | rKKRrMRRr |
| TAT(s2-53) | 104 | 9 | rKKRrQMRr |
| TAT(s2-54) | 105 | 9 | rKKRrQRMr |
| TAT(s2-55) | 106 | 9 | rNKRrQRRr |
| TAT(s2-56) | 107 | 9 | rKNRrQRRr |
| TAT(s2-57) | 108 | 9 | rKKNrQRRr |
| TAT(s2-58) | 109 | 9 | rKKRrNRRr |
| TAT(s2-59) | 110 | 9 | rKKRrQNRr |
| TAT(s2-60) | 111 | 9 | rKKRrQRNr |
| TAT(s2-61) | 112 | 9 | rQKRrQRRr |
| TAT(s2-62) | 113 | 9 | rKQRrQRRr |
| TAT(s2-63) | 114 | 9 | rKKQrQRRr |
| TAT(s2-64) | 115 | 9 | rKKRrKRRr |
| TAT(s2-65) | 116 | 9 | rKKRrQQRr |
| TAT(s2-66) | 117 | 9 | rKKRrQRQr |
| TAT(s2-67) | 118 | 9 | rSKRrQRRr |
| TAT(s2-68) | 119 | 9 | rKSRrQRRr |
| TAT(s2-69) | 120 | 9 | rKKSrQRRr |
| TAT(s2-70) | 121 | 9 | rKKRrSRRr |
| TAT(s2-71) | 122 | 9 | rKKRrQSRr |
| TAT(s2-72) | 123 | 9 | rKKRrQRSr |
| TAT(s2-73) | 124 | 9 | rTKRrQRRr |
| TAT(s2-74) | 125 | 9 | rKTRrQRRr |
| TAT(s2-75) | 126 | 9 | rKKTrQRRr |
| TAT(s2-76) | 127 | 9 | rKKRrTRRr |
| TAT(s2-77) | 128 | 9 | rKKRrQTRr |
| TAT(s2-78) | 129 | 9 | rKKRrQRTr |
| TAT(s2-79) | 130 | 9 | rVKRrQRRr |
| TAT(s2-80) | 131 | 9 | rKVRrQRRr |
| TAT(s2-81) | 132 | 9 | rKKVrQRRr |
| TAT(s2-82) | 133 | 9 | rKKRrVRRr |
| TAT(s2-83) | 134 | 9 | rKKRrQVRr |
| TAT(s2-84) | 135 | 9 | rKKRrQRVr |
| TAT(s2-85) | 136 | 9 | rWKRrQRRr |
| TAT(s2-86) | 137 | 9 | rKWRrQRRr |
| TAT(s2-87) | 138 | 9 | rKKWrQRRr |
| TAT(s2-88) | 139 | 9 | rKKRrWRRr |
| TAT(s2-89) | 140 | 9 | rKKRrQWRr |
| TAT(s2-90) | 141 | 9 | rKKRrQRWr |
| TAT(s2-91) | 142 | 9 | rYKRrQRRr |
| TAT(s2-92) | 143 | 9 | rKYRrQRRr |
| TAT(s2-93) | 144 | 9 | rKKYrQRRr |
| TAT(s2-94) | 145 | 9 | rKKRrYRRr |
| TAT(s2-95) | 146 | 9 | rKKRrQYRr |
| TAT(s2-96) | 147 | 9 | rKKRrQRYr |
| TAT(s2-97) | 148 | 8 | rKKRrQRr |
| TAT(s2-98) | 149 | 9 | rKKRrQRrK |
| TAT(s2-99) | 150 | 9 | rKKRrQRrR |
| r₃R₆ | 151 | 9 | rRRRrRRRr |
| L-R₉ | 152 | 9 | RRRRRRRRR |
| L-R₈ | 153 | 8 | RRRRRRRR |
| L-R₇ | 154 | 7 | RRRRRRR |
| L-R₆ | 155 | 6 | RRRRRR |
| L-R₅ | 156 | 5 | RRRRR |
| r₉ | 157 | 9 | rrrrrrrrr |
| r₅R₄ (D/L) | 158 | 9 | rRrRrRrRr |
| r₅R₄ (DD/LL) | 159 | 9 | rrRRrrRRr |
| PTD-4 | 160 | 11 | YARAAARQARA |
| PTD-4 (variant 1) | 161 | 11 | WARAAARQARA |
| PTD-4 (variant 2) | 162 | 11 | WARAQRAAARA |
| L-P1 Penetratin | 163 | 16 | RQVKVWFQNRRMKWKK |
| D-P1 Penetratin | 164 | 16 | KKWKMRRNQFWVKVQR |
| JNKI, bestfit | 165 | 17 | WKRAAARKARAMSLNLF |
| JNKI, bestfit (variant 1) | 166 | 17 | WKRAAARAARAMSLNLF |
| MDCK transcytose | 167 | 9 | RYRGDLGRR |
| sequence | | | |
| YKGL | 168 | 4 | YKGL |
| P1 | 169 | 4 | RRTK |
| P66 | 170 | 4 | RRPK |

As mentioned above, transporter sequences may also be selected from fragments or variants of the above sequences of table 2 (with the proviso that such fragment or variant retain preferably the function to provide for translocation across biological membranes). In this specific context, variants and/or fragments of those transporter sequences preferably comprise a peptide sequence sharing at least 10%, at least 20%, at least 30%, at least 40%, at least 50%, at least 60%, at least 70%, at least 80% or at least 85%, preferably at least 90%, more preferably at least 95% and most preferably at least 99% sequence identity over the whole length of the sequence with such a transporter sequence as defined in Table 2. In this specific context, a “fragment” of a transporter sequence as defined in Table 2, is preferably to be understood as a truncated sequence thereof, i.e. an amino acid sequence, which is N-terminally, C-terminally and/or intrasequentially truncated compared to the amino acid sequence of the original sequence.

Furthermore, a “variant” of a transporter sequence or its fragment as defined above, is preferably to be understood as a sequence wherein the amino acid sequence of the variant differs from the original transporter sequence or a fragment thereof as defined herein in one or more mutation(s), such as one or more substituted, (or, if necessary, inserted and/or deleted) amino acid(s). Preferably, variants of such a transporter sequence as defined above have the same biological function or specific activity compared to the respective original sequence, i.e. provide for transport, e.g. into cells or the nucleus. In this context, a variant of such a transporter sequence as defined above may for example comprise about 1 to 50, 1 to 20, more preferably 1 to 10 and most preferably 1 to 5, 4, 3, 2 or 1 amino acid alterations. Variants of such a transporter sequence as defined above may preferably comprise conservative amino acid substitutions. The concept of conservative amino acid substitutions is known in the art and has already been set out above for the JNK inhibitory (poly-)peptide sequence and applies here accordingly.

The length of a transporter sequence incorporated in the JNK inhibitor of the present invention may vary. It is contemplated that in some embodiments the transporter sequence of the JNK inhibitor according to the present invention is less than 150, less than 140, less than 130, less than 120, less than 110, less than 100, less than 90, less than 80, less than 70, less than 60, less than 50, less than 40, less than 30, less than 20, and/or less than 10 amino acids in length.

Whether a specific transporter sequence is still functional in the context of the JNK inhibitor according to the present invention may readily be determined by a person skilled in the art. For instance, the JNK inhibitor comprising a transporter domain may be fused to a label, e.g. a fluorescent protein such as GFP, a radioactive label, an enzyme, a fluorophore, an epitope etc. which can be readily detected in a cell. Then, the JNK inhibitor comprising the transporter sequence and the label is transfected into a cell or added to a culture supernatant and permeation of cell membranes can be monitored by using biophysical and biochemical standard methods (for example flow cytometry, (immuno)fluorescence microscopy etc.).

Specific examples of JNK inhibitors according to the present invention comprising a transporter sequence are given in table 3:

TABLE 3
Examples for JNK inhibitors comprising an
inhibitory (poly-)peptide sequence and a
transporter sequence

| Amino acid sequence | AA | SEQ ID NO: |
|---|---|---|
| rKKRrQRRrRPkRPTTLNLf | 20 | 171 |
| rKKRrQRRrRPkRPaTLNLf | 20 | 172 |
| rKKRrQRRrRPkRPTTLrLf | 20 | 173 |
| rKKRrQRRrRPTTLNLf | 17 | 174 |
| rKKRrQRrRPTTLNLf | 16 | 175 |
| rKKRrQRRrRPkRPTTLNLw | 20 | 176 |
| rKKRrQRRrRPkRPTDLNLf | 20 | 177 |
| rKKRrQRRrRPTTLrLw | 17 | 178 |
| rKKRrQRrRPTTLrLw | 16 | 179 |
| rKKRrQRRrRPTDLrLw | 17 | 180 |
| rKKRrQRrRPTDLrLw | 16 | 181 |
| rKKRrQRRrRPaTLNLf | 17 | 182 |
| rKKRrQRrRPaTLNLf | 16 | 183 |
| rKKRrQRrKRPaTLNLf | 17 | 184 |
| rKKRrQRRrRPkRPsTLNLf | 20 | 185 |
| rKKRrQRRrRPkRPqTLNLf | 20 | 186 |
| rKKRrQRRrRPkRPkTLNLf | 20 | 187 |
| rKKRrQRRrGKRKALKLf | 18 | 188 |
| rKKRrQRRrGKRKALrLf | 18 | 189 |
| rKKRrQRRrRKALrLf | 16 | 190 |

As mentioned above, in a particular embodiment of the present invention the transporter sequence and the inhibitory (poly-)peptide sequence may overlap. In other words, the N-terminus of the transporter sequence may overlap with the C-terminus of the inhibitory (poly-)peptide sequence or the C-terminus of the transporter sequence may overlap with the N-terminus of the inhibitory (poly-)peptide sequence. The latter embodiment is particularly preferred. Preferably, the transporter sequence overlaps by one, two or three amino acid residues with the inhibitory (poly-)peptide sequence. In such scenario, a given transporter sequence may overlap with SEQ ID NO:1 or the respective variants thereof at position 1 (X1), position 1 and 2 (X1, X2), positions 1, 2 and 3 (X1, X2, X3).

SEQ ID NOs: 174, 175, 178, 179, 180, 181, 182, 183, 184, 188, 189 and 190 are examples for JNK inhibitors according to the present invention, wherein transporter sequence and the inhibitory (poly-)peptide sequence overlap, e.g.

(SEQ ID NO: 174)
rKKRrQRRrPTTLNLf

is an overlap of SEQ ID NO: 46 (underlined) and SEQ ID NO: 11 (italics).

The JNK inhibitor according to the present invention may also be selected from JNK inhibitors, which are a variant of any one of the JNK inhibitors according to SEQ ID NOs: 171-190. Preferably, such variant shares at least 50%, more preferably at least 55%, more preferably at least 60%, more preferably at least 65%, more preferably at least 70%, more preferably at least 75%, more preferably at least 80%, more preferably at least 85%, more preferably at least 90%, most preferably at least 95% sequence identity with the sequence of SEQ ID NOs: 171-190, in particular with SEQ ID NO: 172,

with the proviso that with respect to the inhibitory (poly-)peptide sequence within said sequences of SEQ ID NOs: 171-190 (see for reference inhibitory (poly-)peptide sequence of SEQ ID NO: 1 and specific examples of SEQ ID NOs: 2-27)) such sequence sharing sequence identity
a) maintains the L-arginine (R) residue on position 4 within the inhibitory (poly-)peptide sequence,
b) maintains the two L-leucine (L) residues at position 8 and 10 (positions 7 and 9 with regard to SEQ ID NOs: 25-27) within the inhibitory (poly-)peptide sequence,
c) exhibits at least one, at least two, at least three, at least four, at least five or six D-amino acid(s) at the respective positions corresponding to the amino acids selected from the group consisting of X1, X2, X3, X5, X7 and or X8 of SEQ ID NO: 1 and respective positions in SEQ ID NOs: 2-27, more preferably exhibits at least one, at least two, at least three or four D-amino acid(s) at the positions corresponding to the amino acids selected from the group consisting of X3, X5, X7 and X8 of SEQ ID NO: 1 and respective positions in SEQ ID NOs: 2-27, and
d) inhibits JNK activity (i.e. is a JNK inhibitor as defined herein).

In view of said definition and for sake of clarity the residues which may preferably not be altered in variants of JNK inhibitors comprising SEQ ID NOs: 171-190 (see a) and b) in the above definition) are underlined in table 3.

The non-identical amino acids in the variants of JNK inhibitors comprising SEQ ID NOs: 171-190 are preferably the result of conservative amino acid substitutions (see above). Certainly, the further possible substitutions mentioned above are also contemplated for variants of JNK inhibitors comprising SEQ ID NOs: 171-190. Likewise, the present invention certainly also contemplates variants of any one of the JNK inhibitors according to SEQ ID NOs: 171-190, which deviate from the original sequence not or not exclusively in the inhibitory (poly-)peptide sequence, but exhibits variant residues in the transporter sequence. For variants and fragments of transporter sequences, the respective disclosure herein is pertinent.

As mentioned previously, the transporter sequence and the JNK inhibitory (poly)-peptide sequence of the JNK inhibitors according to the present invention need not necessarily be directly linked to each other. They may also be linked by e.g. an intermediate or linking (poly-)peptide sequences. Preferred intermediate or linking sequences separating the inhibitory (poly-)peptide sequences and other (functional) sequences such as transporter sequences consist of short peptide sequences of less than 10 amino acids in length, like a hexamer, a pentamer, a tetramer, a tripeptide or a dipeptide or a single amino acid residue. Particularly preferred intermediate sequence are one, two or more copies of di-proline, di-glycine, di-arginine and/or di-lysine, all either in L-amino acid form only, or in D-amino acid form only, or with mixed D- and L-amino acids. Alternatively, other known peptide spacer or linker sequences may be employed as well.

A particularly preferred JNK inhibitor according to the present invention comprises SEQ ID NO: 8 (or a sequence sharing sequence identity with SEQ ID NO: 8 with the scope and limitations defined further above) and a transporter sequence. The transporter sequence is preferably selected from any one of SEQ ID Nos: 31-170 or variants thereof as defined herein, even more preferably from any one of SEQ ID NOs: 31-34 and 46-151. A particularly preferred embodiment of a JNK inhibitor according to the present invention is a JNK inhibitor comprising SEQ ID NO: 8 and SEQ ID NO: 46 (or sequences sharing respective sequence identity thereto within the scope and limitations defined above). A preferred example is a JNK inhibitor comprising the sequence of SEQ ID NO: 172 or respective variants thereof varying in the transporter sequence and/or the inhibitory (poly-)peptide sequence as defined herein.

In a further aspect, the present invention relates to a JNK inhibitor comprising
a) an inhibitory (poly-)peptide comprising a sequence from the group of sequences consisting of RPTTLNLF (SEQ ID NO: 191), KRPTTLNLF (SEQ ID NO: 192), RRPTTLNLF and/or RPKRPTTLNLF (SEQ ID NO: 193), and
b) a transporter sequence, preferably a transporter sequence selected from the transporter sequences disclosed in table 2 or variants/fragments thereof, even more preferably selected from SEQ ID NOs: 31-34 and 46-151 or respective variants or fragments thereof.

The transporter sequence and the inhibitory (poly-)peptide sequence may overlap. Preferred transporter sequences for said embodiment of the invention are particularly the transporter sequence of SEQ ID NO: 46, preferably (covalently) linked (e.g. directly) to the N-terminus of the inhibitory (poly-)peptide sequence.

A JNK inhibitor of the present invention may also be a JNK inhibitor comprising or consisting of the sequence GRKKRRQRRRPPKRPTTLNLFPQVPRSQD (SEQ ID NO: 194), or the sequence GRKKRRQRRRPTTLNLFPQVPRSQD (SEQ ID NO: 195).

In a further aspect, the present invention relates to a (poly-)peptide comprising a transporter sequence selected from the group of sequences consisting of rKKRrQRr (SEQ ID NO: 148), rKKRrQRrK (SEQ ID NO: 149), and/or rKKRrQRrR (SEQ ID NO: 150).

As used herein, “comprising” a sequence or a given SEQ ID NO as disclosed herein usually implies that (at least) one copy of said sequence is present, eg. in the JNK inhibitor molecule. For example, one inhibitory (poly-)peptide sequence will usually suffice to achieve sufficient inhibition of JNK activity. However, it is contemplated according to the invention to use two or more copies of the respective sequence (e.g. two or more copies of an inhibitory (poly-)peptide sequence of different or same type and/or two or more copies of a transporter sequence of different or the same type) may also employed for the inventive (poly)peptide, as long as the overall ability of the resulting molecule to inhibit JNK activity is not abolished (i.e. the respective molecule is still a JNK inhibitor as defined herein).

The inventive JNK inhibitors may be obtained or produced by methods well-known in the art, e.g. by chemical synthesis via solid-phase peptide synthesis using Fmoc (9-fluorenylmethyloxycarbonyl) strategy, i.e. by successive rounds of Fmoc deprotection and Fmoc-amino acid coupling cycles. A commercial service offering such peptide synthesis is provided by many companies, for example the company PolyPeptide (Straβbourg, France).

The JNK inhibitors for use according to the present invention may optionally be further modified, in particular at the amino acid residues of the inhibitory (poly-peptide) sequence. Possible modifications may for example be selected from one or more of items (i) to (xiii) of the group consisting of:
(i) radioactive labels, i.e. radioactive phosphorylation or a radioactive label with sulphur, hydrogen, carbon, nitrogen, etc.;
(ii) colored dyes (e.g. digoxygenin, etc.);
(iii) fluorescent groups (e.g. fluorescein, etc.);
(iv) chemiluminescent groups;
(v) groups for immobilization on a solid phase (e.g. His-tag, biotin, strep-tag, flag-tag, antibodies, epitopes, etc.);
(vi) pegylation,
(vii) glycosylation,
(viii) hesylation,
(ix) protease cleavage sites (e.g. for controlled release of the JNK inhibitor)
(x) peptide backbone modifications (e.g. (ΨCH₂—NH) bonds)
(xi) protection of amino acid side chain residues,
(xii) protection of N- and/or C-terminus (e.g. N-terminal amidation or C-terminal acetylation)
(xiii) a combination of elements of two or more of the elements mentioned under (i) to (xii).

Particularly preferred are modifications selected from (i) to (xi) and combinations of elements of two or more of the elements mentioned under (i) to (xi). In this context, the present invention relates in a further aspect to a JNK inhibitor as disclosed herein modified with modifications selected from (i) to (xi) or modified with a combination of two or more of the elements mentioned under (i) to (xi), and a pharmaceutical composition (see below) comprising such modified JNK inhibitor.

### Pharmaceutical Compositions

The JNK inhibitors as defined according to the invention can be formulated in a pharmaceutical composition, which may be applied in the prevention or treatment of any of the diseases as defined herein. Typically, such a pharmaceutical composition used according to the present invention includes as an active component a JNK inhibitor as defined herein, in particular a JNK inhibitor comprising or consisting of an inhibitory (poly-)peptide sequence according to SEQ ID NO: 1, as defined herein. Preferably, the active compound is a JNK inhibitor comprising or consisting of an inhibitory (poly-)peptide sequence according to any one, of SEQ ID NOs: 2-27, optionally in (covalent) conjugation (via or without a linker sequence) with any suitable transporter sequence; if a transporter sequence is attached, any of the sequences according to any one of SEQ ID NOs: 171-190 may be selected.

The inventors of the present invention additionally found that the JNK-inhibitors as defined herein, in particular if fused to a transporter sequence; exhibit a particularly pronounced uptake rate into cells involved in the diseases of the present invention. Therefore, the amount of a JNK-inhibitor inhibitor in the pharmaceutical composition to be administered to a subject, may—without being limited thereto—be employed on the basis of a low dose within that composition. Thus, the dose to be administered may be much lower than for peptide drugs known in the art, such as DTS-108 (Florence Meyer-Losic et al., Clin Cancer Res., 2008, 2145-53). Thereby, for example a reduction of potential side reactions and a reduction in costs is achieved by the inventive (poly)peptides.

Preferably, the dose (per kg body weight), e.g. to be administered on a daily basis to the subject, is in the range of up to about 10 mmol/kg, preferably up to about 1 mmol/kg, more preferably up to about 100 μmol/kg, even more preferably up to about 10 μmol/kg, even more preferably up to about 1 μmol/kg, even more preferably up to about 100 nmol/kg, most preferably up to about 50 nmol/kg.

Thus, the dose range, e.g. the dose to be administered on a daily basis, may preferably be from about 1 pmol/kg to about 1 mmol/kg, from about 10 pmol/kg to about 0.1 mmol/kg, from about 10 pmol/kg to about 0.01 mmol/kg, from about 50 pmol/kg to about 1 μmol/kg, from about 100 pmol/kg to about 500 nmol/kg, from about 200 pmol/kg to about 300 nmol/kg, from about 300 pmol/kg to about 100 nmol/kg, from about 500 pmol/kg to about 50 nmol/kg, from about 750 pmol/kg to about 30 nmol/kg, from about 250 pmol/kg to about 5 nmol/kg, from about 1 nmol/kg to about 10 nmol/kg, or a combination of any two of said values.

In this context, prescription of treatment, e.g. decisions on dosage etc. when using the above pharmaceutical composition is typically within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in REMINGTON'S PHARMACEUTICAL SCIENCES, 16th edition, Osol, A. (ed), 1980. Accordingly, a “safe and effective amount” for components of the pharmaceutical compositions as used according to the present invention means an amount of each or all of these components, that is sufficient to significantly induce a positive modification of diseases or disorders strongly related to JNK signalling as defined herein. At the same time, however, a “safe and effective amount” is small enough to avoid serious side-effects, that is to say to permit a sensible relationship between advantage and risk. The determination of these limits typically lies within the scope of sensible medical judgment. A “safe and effective amount” of such a component will vary in connection with the particular condition to be treated and also with the age and physical condition of the patient to be treated, the severity of the condition, the duration of the treatment, the nature of the accompanying therapy, of the particular pharmaceutically acceptable carrier used, and similar factors, within the knowledge and experience of the accompanying doctor. The pharmaceutical compositions according to the invention can be used according to the invention for human and also for veterinary medical purposes.

The pharmaceutical composition as used according to the present invention may furthermore comprise, in addition to one or more of the JNK inhibitors, a (compatible) pharmaceutically acceptable carrier, excipient, buffer, stabilizer or other materials well known to those skilled in the art.

In this context, the expression “(compatible) pharmaceutically acceptable carrier” preferably includes the liquid or non-liquid basis of the composition. The term “compatible” means that the constituents of the pharmaceutical composition as used herein are capable of being mixed with the pharmaceutically active component as defined above and with one another component in such a manner that no interaction occurs which would substantially reduce the pharmaceutical effectiveness of the composition under usual use conditions. Pharmaceutically acceptable carriers must, of course, have sufficiently high purity and sufficiently low toxicity to make them suitable for administration to a person to be treated.

If the pharmaceutical composition as used herein is provided in liquid form, the pharmaceutically acceptable carrier will typically comprise one or more (compatible) pharmaceutically acceptable liquid carriers. The composition may comprise as (compatible) pharmaceutically acceptable liquid carriers e.g. pyrogen-free water; isotonic saline or buffered (aqueous) solutions, e.g. phosphate, citrate etc. buffered solutions, vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid, etc. Particularly for injection of the pharmaceutical composition as used herein, a buffer, preferably an aqueous buffer, may be used.

If the pharmaceutical composition as used herein is provided in solid form, the pharmaceutically acceptable carrier will typically comprise one or more (compatible) pharmaceutically acceptable solid carriers. The composition may comprise as (compatible) pharmaceutically acceptable solid carriers e.g. one or more compatible solid or liquid fillers or diluents or encapsulating compounds may be used as well, which are suitable for administration to a person. Some examples of such (compatible) pharmaceutically acceptable solid carriers are e.g. sugars, such as, for example, lactose, glucose and sucrose; starches, such as, for example, corn starch or potato starch; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulphate, etc.

The precise nature of the (compatible) pharmaceutically acceptable carrier or other material may depend on the route of administration. The choice of a (compatible) pharmaceutically acceptable carrier may thus be determined in principle by the manner in which the pharmaceutical composition as used according to the invention is administered. The pharmaceutical composition as used according to the invention can be administered, for example, systemically. Routes for administration include, for example, parenteral routes (e.g. via injection), such as intravenous, intramuscular, subcutaneous, intradermal, or transdermal routes, etc., enteral routes, such as oral, or rectal routes, etc., topical routes, such as nasal, or intranasal routes, etc., or other routes, such as epidermal routes or patch delivery. Also contemplated (in particular for eye related diseases) are instillation, intravitreal, and subconjunctival administration. Likewise, administration may occur intratympanical, for example, whenever ear related diseases are treated.

The suitable amount of the pharmaceutical composition to be used can be determined by routine experiments with animal models. Such models include, without implying any limitation, rabbit, sheep, mouse, rat, dog and non-human primate models. Preferred unit dose forms for injection include sterile solutions of water, physiological saline or mixtures thereof. The pH of such solutions should be adjusted to about 7.4. Suitable carriers for injection include hydrogels, devices for controlled or delayed release, polylactic acid and collagen matrices. Suitable pharmaceutically acceptable carriers for topical application include those, which are suitable for use in lotions, creams, gels and the like. If the compound is to be administered per orally, tablets, capsules and the like are the preferred unit dose form. The pharmaceutically acceptable carriers for the preparation of unit dose forms, which can be used for oral administration are well known in the prior art. The choice thereof will depend on secondary considerations such as taste, costs and storability, which are not critical for the purposes of the present invention, and can be made without difficulty by a person skilled in the art.

Pharmaceutical compositions for oral administration may be in tablet, capsule, powder or liquid form. A tablet may include a solid carrier as defined above, such as gelatin, and optionally an adjuvant. Liquid pharmaceutical compositions for oral administration generally may include a liquid carrier as defined above, such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required. Whether it is a polypeptide, peptide, or nucleic acid molecule, other pharmaceutically useful compound according to the present invention that is to be given to an individual, administration is preferably in a “prophylactically effective amount or a “therapeutically effective amount” (as the case may be), this being sufficient to show benefit to the individual. The actual amount administered, and rate and time-course of administration, will depend on the nature and severity of what is being treated.

Treatment of a disease as defined herein typically includes administration of a pharmaceutical composition as defined above. The JNK inhibitors of the present invention will modulate the JNK activity in the subject. The term “modulate” includes in particular the suppression of phosphorylation of c-jun, ATF2 or NFAT4 in any of the above diseases, for example, by using at least one JNK inhibitor comprising or consisting of an inhibitory (poly)peptide sequence according to any of sequences of SEQ ID NOs: 2 to 27, potentially comprising an additional transporter sequence, as a competitive inhibitor of the natural c-jun, ATF2 and NFAT4 binding site in a cell. The term “modulate” also includes suppression of hetero- and homomeric complexes of transcription factors made up of, without being limited thereto, c-jun, ATF2, or NFAT4 and their related partners, such as for example the AP-1 complex that is made up of c-jun, AFT2 and c-fos.

Treatment of a subject with the pharmaceutical composition as disclosed above may be typically accomplished by administering (in vivo) an (“therapeutically effective”) amount of said pharmaceutical composition to a subject, wherein the subject may be e.g. a human subject or an animal. The animal is preferably a non-human mammal, e.g., a non-human primate, mouse, rat, dog, cat, cow, horse or pig. The term “therapeutically effective” means that the active component of the pharmaceutical composition is of sufficient quantity to ameliorate the diseases and disorders as discussed herein.

### Diseases and Disorders

The present invention is directed to specific uses (or methods of use) of the above disclosed JNK inhibitors or pharmaceutical compositions containing the same in a method for treatment of the human or animal body by therapy, in particular of the human body. As mentioned above JNK signalling is involved in a multitude of diverse disease states and disorder and inhibition of said signalling has proposed and successfully tested for many of these. The inventors of the present invention found that the JNK inhibitors disclosed herein are effective JNK inhibitors for the treatment of the diseases as disclosed in the following.

Treatment of a human or animal body by therapy, as used herein, refers to any kind of therapeutic treatment of a respective subject. It includes for example prevention of onset of the disease or symptoms (prophylaxis), i.e. typically prior to manifestation of the disease in the patient. The term also includes the “mere” treatment of symptoms of a given disease, i.e. the treatment will ameliorate pathogenesis by reducing disease-associated symptoms, without necessarily curing the underlying cause of the disease and symptoms. Certainly, curing the underlying cause of the disease is also encompassed by the term. The term also encompasses a treatment which delays or even stops progression of the respective disease.

In one embodiment the JNK inhibitors according to the present invention may be administered for example prophylactically prior to potential onset of a foreseeable disorder, e.g. prior to a planned surgical intervention or planned exposure to stressful stimuli. A surgical intervention could for example bear the risk of inflammation of the respective wound or neighbouring tissue. Exposure to stressful stimuli like radiation could lead to apoptosis of affected tissue and cells. In such scenario, the JNK inhibitors according to the present invention may, for example, be administered at least once up to about 4 weeks in advance. The JNK inhibitors may for example be administered at least 24 hours, at least 48 hours, at least 1 week, at least 2 weeks or 4 weeks in advance.

The diseases and disorders to be treated with the JNK inhibitors as disclosed herein may be acute or chronic.

While the JNK inhibitors of the present invention may be used in general for the treatment of diseases of various organs, such as diseases of the eye, diseases of the bone, neural diseases, neuronal diseases, neurodegenerative diseases, diseases of the skin, immune and/or autoimmune diseases, diseases of the eye, diseases of the mouth, inflammatory diseases, metabolic diseases, cardiovascular diseases, proliferative diseases (in particular cancers and tumors), diseases of the ear, diseases of the intestine, diseases of the respiratory system (e.g. lung diseases), infectious diseases, and various other diseases, the present invention specifically refers to the following diseases:

Among the disease to be treated by the inventive molecules, skin diseases, in particular inflammatory skin diseases, more specifically skin diseases selected from the group consisting of eczema, Psoriasis, dermatitis, acne, mouth ulcers, erythema, Lichen plan, sarcoidosis, vascularitis and adult linear IgA disease, are to be mentioned. Dermatitis encompasses e.g. atopic dermatitis or contact dermatitis.

(Anti-inflammatory) treatment upon tissue or organ transplantation, is treatable by the inventive molecules in particular upon heart, kidney, and skin (tissue), lung, pancreas, liver, blood cells (e.g. any kind of blood cell, such as platelets, white blood cells, red blood cells), bone marrow, cornea, accidental severed limbs (fingers, hand, foot, face, nose etc.), bones of whatever type, cardiac valve, blood vessels, segments of the intestine or the intestine as such. Such a treatment is e.g. considered appropriate whenever e.g. a graft vs. host or host vs graft reaction occurs upon organ/tissue transplantation. The use of the inventive molecules may also be employed whenever transplantation surgery is carried, in particular in case of skin (or, pancreas, liver, lung, heart, kidney) graft vs. host or host vs. skin (or, pancreas, liver, lung, heart, kidney) graft reaction.

Among neurodegenerative diseases, in particular those associated with chronic inflammation, tauopathies and amyloidoses and prion diseases are addressed by the inventive molecules. Other such neurodegenerative disease refer to the various forms of dementia, e.g. frontotemporal dementia and dementia with lewy bodies, schizophrenia and bipolar disorder, spinocerebellar ataxia, spinocerebellar atrophy, multiple system atrophy, motor neuron disease, corticobasal degeneration, progressive supranuclear palsy or hereditary spastic paraparesis. Another field of indication is pain (e.g. neuropathic, incident, breakthrough, psychogenic, phantom, chronic or acute forms of pain). Another field of use is the treatment of bladder diseases, in particular for treating loss of bladder function (e.g. urinary incontinence, overactive bladder, interstitial cystitis or bladder cancer) or stomatitis.

The inventive molecules are used for the treatment of fibrotic diseases or fibrosis as well, in particular lung, heart, liver, bone marrow, mediastinum, retroperitoneum, skin, intestine, joint, and shoulder fibrosis.

While inflammatory diseases of the mouth and the jaw/mandible are treatable in general by the inventive molecules, gingivitis, osteonecrosis (e.g. of the jaw bone), peri-implantitis, pulpitis, and periodontitis are particularly suitable for the use of these inventive molecules for therapeutic purposes.

In addition, polypes are effectively treatable by using the inventive molecules.

Also inflammatory or non-inflammatory pathophysiologies of the kidney are effectively treated by using the inventive molecules. In particular, the disease is selected from the group consisting of glomerulonephritis in general, in particular membrano-proliferative glomerulonephritis, mesangio-proliferative glomerulonephritis, rapidly progressive glomerulonephritis, nephrophathies in general, in particular membranous nephropathy or diabetic nephropathy, nephritis in general, in particular lupus nephritis, pyelonephritis, interstitial nephritis, tubulointerstitial nephritis, chronic nephritis or acute nephritis, and minimal change disease and focal segmental glomerulosclerosis.

Among the diseases or disorders which are effectively treated by the inventive molecules, a larger number of diseases or disorders may be linked to inflammatory processes, but do not necessarily have to be associated with such inflammatory processes. The following diseases or disorders are specifically disclosed in this regard as being treatable by the use of the inventive molecules: Addison's disease, Agammaglobulinemia, Alopecia areata, Amytrophic lateral sclerosis, Antiphospholipid syndrome, Atopic allergy, Autoimmune aplastic anemia, Autoimmune cardiomyopathy, Autoimmune enteropathy, Autoimmune hemolytic anemia, Autoimmune inner ear, disease, Autoimmune lymphoproliferative syndrome, Autoimmune polyendocrine syndrome, Autoimmune progesterone dermatitis, Idiopathic thrombocytopenic purpura, Autoimmune urticaria, Balo concentric sclerosis, Bullous pemphigoid, Castleman's disease, Cicatricial pemphigoid, Cold agglutinin disease, Complement component 2 deficiency associated disease, Cushing's syndrome, Dagos disease, Adiposis dolorosa, Eosinophilic pneumonia, Epidermolysis bullosa acquisita, Hemolytic disease of the newborn, Cryoglobulinemia, Evans syndrome, Fibrodysplasia ossificans progressive, Gastrointestinal pemphigoid, Goodpasture's syndrome, Hashimoto's encephalopathy, Gestational pemphigoid, Hughes-stovin syndrome, Hypogammaglobulinemia, Lambert-eaton myasthenic syndrome, Lichen sclerosus, Morphea, Pityriasis lichenoides et varioliformis acuta, Myasthenia gravis, Narcolepsy, Neuromyotonia, Opsoclonus myoclonus syndrome, Paraneoplastic cerebellar degeneration, Paroxysmal nocturnal hemoglobinuria, Parry-romberg syndrome, Pernicious anemia, POEMS syndrome, Pyoderma gangrenosum, Pure red cell aplasia, Raynaud's phenomenon, Restless legs syndrome, Retroperitoneal fibrosis, Autoimmune polyendocrine syndrome type 2, Stiff person syndrome, Susac's syndrome, Febrile neutrophilic dermatosis, Sydenham's chorea, Thrombocytopenia, and vitiligo.

While any kind of inflammatory eye disease may be treated by the use of the inventive molecules, the following eye-related diseases are specifically disclosed: inflammation after corneal surgery, non-infective keratitis, chorioretinal inflammation, and sympathetic ophthalmia.

A further class of inflammatory-associated diseases to be treated by the use of the inventive molecules is the following: acute disseminated encephalomyelitis, antisynthetase syndrome, autoimmune hepatitis, autoimmune peripheral neuropathy, pancreatitis, in particular autoimmune pancreatitis, Bickerstaff's encephalitis, Blau syndrome, Coeliac disease, Chagas disease, chronic inflammatory demyelinating polyneuropathy, osteomyelitis, in particular chronic recurrent multifocal osteomyelitis, Churg-Strauss syndrome, Cogan syndrome, giant-cell arteritis, CREST syndrome, vasculitis, in particular cutaneous small-vessel vasculitis or urticarial vasculitis, dermatitis, in particular dermatitis herpetiformis, dermatomyositis, systemic scleroderma, Dressler's syndrome, drug-induced lupus erythematosus, discoid lupus erythematosus, enthesitis, eosinophilic fasciitis, gastroenteritis, in particular, eosinophilic gastroenteritis, erythema nodosum, idiopathic pulmonary fibrosis, gastritis, Grave's disease, Guillain-barré syndrome, Hashimoto's thyroiditis, Henoch-Schonlein purpura, Hidradenitis suppurativa, idiopathic inflammatory demyelinating diseases, myositis, in particular inclusion body myositis, cystitis, Kawasaki disease, Lichen planus, lupoid hepatitis, Majeed syndrome, Ménière's disease, Microscopic polyangiitis, mixed connective tissue disease, myelitis, in particular neuromyelitis, e.g. neuromyelitis optica, thyroiditis, in particular Ord's thyroiditis, rheumatism, in particular palindromic rheumatism, Parsonage-Turner syndrome, perivenous encephalomyelitis, polyarteritis nodosa, polymyalgia rheumatica, polymyositis, cirrhosis, in particular primary biliary cirrhosis, cholangitis, in particular primary sclerosing cholangitis, progressive inflammatory neuropathy, Rasmussen's encephalitis, chondritis, in particular polychondritis, e.g. relapsing polychondritis, reactive arthritis (Reiter disease), rheumatic fever, sarcoidosis, Schnitzler syndrome, serum sickness, spondylitis, in particular ankylosing spondylitis, spondyloarthropathy, Takayasu's arteritis, Tolosa-Hunt syndrome, transverse myelitis, and granulomatosis, in particular Wegener's granulomatosis.

In the most preferred embodiment of the present invention, the inventive molecules are used for the treatment of the following diseases or disorders: psoriasis, dry eye disease, persistent or acute inflammatory diseases damaging the retina of the eye (retinopathy), in particular diabetic retinopathy or retinopathies caused by other diseases, age-related macular degeneration (AMD), in particular the wet or the dry form of age-related macular degeneration, retinopathy of prematurity (ROP), persistent or acute inflammatory diseases of the mouth, in particular peri-implantitis, pulpitis, periodontitis, anti-inflammatory treatment upon tissue or organ transplantation, in particular upon heart, kidney, and skin (tissue) transplantation, graft rejection upon heart, kidney or skin (tissue) transplantation, inflammatory brain diseases, in particular for the treatment of Alzheimer's disease, metabolic disorders, glomerulonephritis, and arthrosis/arthritis, in particular reactive arthritis, rheumatoid arthrosis, juvenile idiopathic arthritis, and psoriatic arthritis.

The “dry” form of advanced AMD, results from atrophy of the retinal pigment epithelial layer below the retina, which causes vision loss through loss of photoreceptors (rods and cones) in the central part of the eye. Neovascular, the “wet” form of advanced AMD, causes vision loss due to abnormal blood vessel growth (choroidal neovascularization) in the choriocapillaris, through Bruch's membrane, ultimately leading to blood and protein leakage below the macula. Bleeding, leaking, and scarring from these blood vessels eventually cause irreversible damage to the photoreceptors and rapid vision loss, if left untreated. The inventive molecules are suitable for treating both forms of AMD.

Retinopathy of prematurity (ROP), previously known as retrolental fibroplasia (RLF), is a disease of the eye affecting prematurely-born babies generally having received intensive neonatal care. It is thought to be caused by disorganized growth of retinal blood vessels which may result in scarring and retinal detachment. ROP can be mild and may resolve spontaneously, but it may lead to blindness in serious cases. As such, all preterm babies are at risk for ROP, and very low birth weight is an additional risk factor. Both oxygen toxicity and relative hypoxia can contribute to the development of ROP. The inventive molecules are suitable for treating ROP.

Furthermore, the inventive molecules are particularly suitable to treat all forms of retinopathy, in particular diabetes mellitus induced retinopathy, arterial hypertension induced hypertensive retinopathy, radiation induced retinopathy (due to exposure to ionizing radiation), sun-induced solar retinopathy (exposure to sunlight), trauma-induced retinopathy (e.g. Purtscher's retinopathy) and hyperviscosity-related retinopathy as seen in disorders which cause paraproteinemia).

The JNK inhibitors of the present invention may also be used for the treatment of metabolic disorders, for example for the treatment of diabetes (type 1 or type 2, in particular type 1), Fabry disease, Gaucher disease, hypothermia, hyperthermia hypoxia, lipid histiocytosis, lipidoses, metachromatic leukodystrophy, mucopolysaccharidosis, Niemann-Pick disease, obesity, and Wolman's disease. More generally, metabolic disorders may be of hereditary form or may be acquired disorders of carbohydrate metabolism, e.g., glycogen storage disease, disorders of amino acid metabolism, e.g., phenylketonuria, maple syrup urine disease, glutaric acidemia type 1, Urea Cycle Disorder or Urea Cycle Defects, e.g., Carbamoyl phosphate synthetase I deficiency, disorders of organic acid metabolism (organic acidurias), e.g., alcaptonuria, disorders of fatty acid oxidation and mitochondrial metabolism, e.g., Medium-chain acyl-coenzyme A dehydrogenase deficiency (often shortened to MCADD.), disorders of porphyrin metabolism, e.g. acute intermittent porphyria, disorders of purine or pyrimidine metabolism, e.g., Lesch-Nyhan syndrome, Disorders of steroid metabolism, e.g., lipoid congenital adrenal hyperplasia, or congenital adrenal hyperplasia, disorders of mitochondrial function, e.g., Kearns-Sayre syndrome, disorders of peroxisomal function. e.g., Zellweger syndrome, or Lysosomal storage disorders, e.g., Gaucher's disease or Niemann Pick disease.

A person skilled in the art will readily realize that the above mentioned disease states and disorders may belong to more than one of the above mentioned disease classes. For example, bronchial carcinoma is certainly not only a proliferative disease but would also belong in the group of diseases of the respiratory system including lung diseases. Thus, the above mentioned classification of individual diseases is not considered to be limiting or concluding but is considered to of exemplary nature only. It does not preclude that individual disease states recited in one class are factually also suitable examples for the application of the JNK inhibitors of the present invention as treatment in another class of disease states. A person skilled in the art will readily be capable of assigning the different disease states and disorders to matching classifications.

Finally, as mentioned above, the present invention contemplates the use of a JNK inhibitor as defined herein for the treatment of various diseases states and disorders. The present invention does not contemplate to use the JNK inhibitors as defined herein for immunizing non-human animals, e.g. for the production of monoclonal antibodies. Such methods are herein not considered to be methods for treatment of the animal body by therapy.

All references cited herein are herewith incorporated by reference.

### EXAMPLES

In the following, particular examples illustrating various embodiments and aspects of the invention are presented. However, the present invention shall not to be limited in scope by the specific embodiments described herein. Indeed, various modifications of the invention in addition to those described herein will become readily apparent to those skilled in the art from the foregoing description, accompanying figures and the examples below. All such modifications fall within the scope of the appended claims.

### Example 1

### Synthesis of JNK Inhibitor SEQ ID NO: 172

As illustrative example, synthesis of the JNK inhibitor with SEQ ID NO: 172 is set out below. A person skilled in the art will know that said synthesis may also be used for and easily adapted to the synthesis of any other JNK inhibitor according to the present invention.

The JNK inhibitor with SEQ ID NO: 172 was manufactured by solid-phase peptide synthesis using the Fmoc (9-fluorenylmethyloxycarbonyl) strategy. The linker between the peptide and the resin was the Rink amide linker (p-[Fmoc-2,3-dimethoxybenzyl]-phenoxyacetic acid). The peptide was synthesized by successive Fmoc deprotection and Fmoc-amino acid coupling cycles. At the end of the synthesis, the completed peptide was cleaved by trifluoroacetic acid (TFA) directly to yield the crude C-terminal amide, which was then purified by preparative reverse phase HPLC. The purified fractions were pooled in a homogeneous batch that is treated by ion exchange chromatography to obtain its acetate salt. The peptide was then freeze-dried.

### 1.1 Solid Phase Synthesis of the Peptide

Except when noted, the manufacturing took place at room temperature (22° C.±7° C.) in an air-filtered environment. The scale of synthesis was 0.7 mmoles of the starting amino acid on the resin, for an expected yield of about 1 g of purified peptide. Synthesis was performed manually in a 30-50 mL reactor equipped with a fritted disk with mechanical stirring and/or nitrogen bubbling.

### 1.2 Preparation of the Resin

The p-methylbenzhydrylamide resin (MBHA-resin) was first washed with dichloromethane/dimethylformamide/diisoproplyethylamine under nitrogen. The washed resin was then coupled to the Rink amide linker (p-[Fmox-2,4-dimethoxybenzyl]-phenoxyacetic acid) in PyBOB (benzotriazole-1-yl-oxy-tris-pyrrolidino-phosphonium hexafluorophosphate)/diisopropylethylamine/1-hydroxybenzotriazole to yield Fmoc-Rink amide-MBHA resin.

### 1.3 Coupling of Amino Acids

Amino acids were coupled to the resin using the following cycle: The Fmoc-Rink amide-MBHA resin was deprotected by washing it in 35% (v/v) piperidine/dimethylformamide, followed by dimethylformamide. The deprotection reaction took approximately 16 minutes. Fmoc-protected amino acids (e.g., 2 eq of amino acid and HOBt (1-hydroxybenzotriazole) in dimethylformamide/dichloromethane (50/50) were added to the resin followed by addition of 2 eq of the coupling agent diisopropylcarbodiimide (DIC). The coupling reaction took from one hour to overnight depending upon the respective amino acid being added. Volumes were calculated on a basis of 0.5 mL/100 mg of peptide-resin and adjusted after each cycle. After coupling, the resin was washed 3 times with DMF. Completeness of coupling was tested by the ninhydrin test (or Kaiser test 1) on primary amines and the chloranyl test 2 on secondary amines. On some occasions, the chloranyl test may be associated with a ninhydrin test as a security control. In case the coupling test indicated incompleteness of reaction, coupling was repeated with a lower excess (0.5-1 eq) of amino acid, PYBOP, HOBT in dimethylformamide/dichloromethane and diisopropylethylamine. Functionality of the resin was measured and generally 0.6-0.2 meq/g, depending on the original loading of the resin. After the last amino acid has been coupled, the peptide-resin was deprotected as usual and then washed 5 times with DCM before drying in an oven under vacuum at 30° C. After the peptide-resin had dried, the yield of the solid-phase synthesis was calculated as the ratio of the weight increase of the peptide resin compared to the theoretical weight increase calculated from the initial loading of the resin. The yield may be close to 100%.

### 1.4 Cleavage And Deprotection

The peptide was cleaved from the resin in a mixture of trifluoroacetic acid/1,2-ethanedthiol/thioanisole/water/phenol (88/2.2/4.4/4.4/7 v/v), also called TFA/K reagent, for 4 hours at room temperature. The reaction volume was 1 mL/100 mg of peptide resin. During addition of the resin to the reagent, the mixture temperature was regulated to stay below 30° C.

1.5 Extraction of the Peptide from the Resin:

The peptide was extracted from the resin by filtration through a fritted disc. After concentration on a rotavapor to ⅓ of its volume, the peptide was precipitated by cold t-butyl methyl ether and filtered. The crude peptide was then dried under vacuum at 30° C.

### 1.6 Preparative HPLC Purification:

The crude peptide was then purified by reverse-phase HPLC to a purity of ≧95%. The purified fractions were concentrated on a rotavaporator and freeze-dried.

### 1.7 Ion Exchange Chromatography

The concentrated freeze-dried pools of purified peptide with the sequence of SEQ ID NO: 172 was dissolved in water and purified by ion exchange chromatography on Dowex acetate, 50-100 mesh resin.

The required starting reagents for the synthesis were:

| | CAS Registry | | Molecular |
|---|---|---|---|
| | Number | Chemical Name | Weight |
| Fmoc-Rink amide linker | 145069-56-3 | p-[Fmoc-2,4-dimethoxybenzyl]- | 539.6 |
| | | phenoxyacetic acid | |
| Fmoc-D-Ala-OH, H₂O | 79990-15-1 | N-alpha-Fmoc-D-alanine | 311.3 |
| Fmoc-Arg(Pbf)-OH | 154445-77-9 | N-alpha-Fmoc-N [2,2,4,6,7- | 648.8 |
| | | pentamethyldihydrobenzofuran-5- | |
| | | sulfonyl]-arginine | |
| Fmoc-D-Arg(Pbf)-OH | 187618-60-6 | N-alpha-Fmoc-N [2,2,4,6,7- | 648.8 |
| | | pentamethyldihydrobenzofuran-5- | |
| | | sulfonyl]-D-arginine | |
| Fmoc-Asn(Trt)-OH | 132388-59-1 | N-alpha-Fmoc-N--trityl-asparagine | 596.7 |
| Fmoc-Gln(Trt)-OH | 132327-80-1 | N-alpha-Fmoc-N--trityl-glutamine | 610.7 |
| Fmoc-Leu-OH | 35661-60-0 | N-alpha-Fmoc-leucine | 353.4 |
| Fmoc-Lys(Boc)-OH | 71989-26-9 | N-alpha-Fmoc-N-Boc-lysine | 468.5 |
| Fmoc-D-Lys(Boc)-OH | 143824-78-6 | N-alpha-Fmoc-N-Boc-D-lysine | 468.5 |
| Fmoc-D-Phe-OH | 86123-10-6 | N-alpha-Fmoc-D-phenylalanine | 387.4 |
| Fmoc-Pro-OH | 71989-31-6 | N-alpha-Fmoc-proline | 337.4 |
| Fmoc-Thr(tBu)-OH | 71989-35-0 | N-alpha-Fmoc-O-t-butyl-threonine | 397.5 |

Other JNK inhibitors of the present invention may be prepared in similar manner.

### Example 2

### Inhibitory Efficacy of Selected JNK Inhibitors According to the Present Invention

In the following a standard operating procedure will be set forth describing how the Inhibitory efficacy of JNK inhibitors according to the present invention was measured. The method allows to measure in vitro, in a non radioactive standardized assay, the ability of a candidate compound to decrease the phosphorylation of the c-Jun specific substrate by JNK. Moreover, it will be illustrated how to determine the inhibitory effect (IC50) and the Ki of a chosen compound for JNK. The method is suitable to verify whether a candidate compound does or does not inhibit JNK activity. and a person skilled in the art will certainly understand how to adapt the below methods for his specific purposes and needs.

### 2.1 Material

### AlphaScreen Reagent and Plate:

His-JNK1 (ref 14-327, Upstate, 10 μg in 100 μl: concentration: 2.2 μM) 5 nM final
His-JNK2 (ref 14-329, Upstate, 10 μg in 100 μl: concentration: 2 μM) 5 nM final
His-JNK3 (ref 14-501, Upstate, 10 μg in 100 μl: concentration: 1.88 μM) 5 nM final
Anti-Phospho-cJun (ref 06-828, Upstate, lot DAM1503356, concentration: 44.5 μM) 10 nM final
Biotin-cJun (29-67):
sequence: Biotin—SNPKILKQSMTLNLADPVGSLKPHLRAKNSDLLTSPDVG (SEQ ID NO: 198), lot 100509 (mw 4382.11, P 99.28%) dissolved in H₂O, concentration: 10 mM) 30 nM final
ATP (ref AS001A, Invitrogen, lot 50860B, concentration 100 mM)) 5 μM final
SAD beads (ref 6760617M, PerkinElmer, lot 540-460-A, concentration 5 mg/ml) 20 μg/ml final
AprotA beads (ref 6760617M, PerkinElmer, lot 540-460-A, concentration 5 mg/ml) 20 μg/ml final
Optiplate 384 well white plate (ref 6007299, PerkinElmer, lot 654280/2008)
96 well plate for peptide dilution (ref 82.1581, Sarstedt)
TopSeals-A (ref 6005185, Perkin Elmer, Lot 65673)
Bioluminescent energy transfer reading
The bioluminescent energy transfer was read on the Fusion Alpha Plate reader (Perkin Elmer).

### Pipette:

An electronic EDP3 pipette 20-300 (Ref 17007243; Rainin) was used to fill in the plate with the Enzyme-Antibody mix, the Substrate-ATP mix and the Beads.
A PIPETMAN® Ultra multichannel 8×20 (Ref 21040; Gilson) was used to fill in the plate with the inhibitory compounds.

### Buffer and Solutions

Kinase Buffer: 20 mM Tris-base pH 7.4, 10 mM MgCl₂, 1 mM DTT, 100 μM Na₃VO₄, 0.01% Tween, (1% DMSO)
Stop Buffer: 20 mM Tris-base pH 7.4, 200 mM NaCl, 80 mM EDTA-K (pH de 8 with KOH instead of NaOH), 0.3% BSA
JNK dilution Kinase buffer: 50 mM Tris-base pH 7.4, 150 mM NaCl, 0.1 mM EGTA, 0.03% Brij-35, 270 mM sucrose, 0.1% β-mercaptoethanol.

### 2.2 Method

To assess inhibitory effect of the peptides, a standard AlphaScreen assay (see for example Guenat et al. J Biomol Screen, 2006; 11: pages 1015-1026) was performed. The different components were prepared and subsequently mixed as indicated. The plates were sealed and incubated as following:

| | | |
|---|---|---|
| 5 μl | JNK + Antibody | |
| 5 μl | TP kinase +/− inhibitor | Pre-incubation 30 min |
| 5 μl | Biotin-cJun + ATP | Incubation 60 min at 24° C. |
| 10 μl | Beads SAD + A protA | Incubation 60 min in the dark at |
| | | 24° C. |

To avoid contamination, the mixes were added with the pipette in different corner of the well. After the filling in of the plate with each mix, the plate was tapped (Keep one side fix and let the opposite side tap the table) to let the mix go down the walls of the wells.

The bioluminescent energy transfer was read on the Fusion Alpha Plate reader (Perkin Elmer).

All compounds should at least be tested in triplicate in 3 independent experiments for each isoform of JNK. Possibly concentrations of the compounds to be tested were 0, 0.03 nM, 0.1 nM, 0.3 nM, 1 nM, 3 nM, 10 nM, 30 nM, 100 nM, 300 nM, 1 μM, 3 μM, 10 μM, 30 μM, and 100 μM. Controls were samples either without JNK or without substrate (c-Jun).

### Mix Preparation

### JNK1, JNK2 and JNK3 5 nM

### Biotin-cJun 30 nM

ATP 5 μM; Anti phospho-cJun (S63) 10 nM
Bille SAD/AprotA 20 μg/ml

Antibody [final]=10 nM (anti Phospho dun (S63))

Detection part: [Mix]×5 (5 μl in final volume of 25 μl)

### [Stock]=44.5 μM (ref 06-828, Upstate, Lot DAM1503356)

10 nM→50 nM in Kinase Buffer
JNK1, JNK2 and JNK3 [final]=5 nM
Reaction part: [Mix]×3 (5 μl in final volume of 15 μl)

### [Stock]=2.2 μM for JNK1 (ref 14-327, Upstate, lot D7KN022CU)

2.0 μM for JNK2 (ref 14-329, Upstate, lot 33221CU)
1.88 μM for JNK3 (ref 14-501, Upstate, lot D7CN041 CU)
   5 nM→15 nM in Antibody Buffer

### Inhibitor:

Reaction part: [Mix]×3 (5 μl in final volume of 15 μl)

### [Stock]=10 mM

| | | | |
|---|---|---|---|
| 100 | μM | → | 300 μM in Kinase Buffer |
| 30 | μM | → | 90 μM in Kinase Buffer |
| 10 | μM | → | 30 μM in Kinase Buffer |

. . .

| | | | |
|---|---|---|---|
| 0.03 | nM | → | 0.09 nM in Kinase Buffer |
| And 0 | nM | → | Kinase Buffer |

Two series of 10 times serial dilutions were performed in a 96 well plate, one beginning with 300 μM to 0 nM, the second with 90 μM to 0.03 nM. The peptides are added in the 384 plates with an 8 channels multipipette (ref F14401, Gilson, 8×20).

ATP [final]=5 μM
Reaction part: [Mix]×3 (5 μl in final volume of 15 μl)

### [Stock]=100 mM (ref AS001A, Invitrogen, lot 50860B)

### 5 μM→15 μM in Kinase Buffer

Biotin c-Jun [final]=30 nM
Reaction part: [Mix]×3 (5 μl in final volume of 15 μl)

### [Stock]=10 mM

30 nM→30 nM in ATP Buffer
Beads SAD/A ProtA [final]=20 μg/ml (Light sensitive)
Detection part: [Mix]×2.5 (10 μl in final volume of 25 μl)
[Stock]=5 mg/ml→20 μg/ml 50 μg/ml in STOP Buffer
Mix in the dark room (green Light) or in the darkness.

### Analysis of the IC50 Curves:

The analysis was performed by the GraphPad Prism4 software with the following equation: Sigmoidal dose-response (No constraint).

Y=Bottom+(Top−Bottom)/(1+10̂((Log EC50−X)))

The outliers data were avoided using Grugg's test.

### Comparison of the IC50:

The analysis was performed by the GraphPad Prism4 software with the following test: One way ANOVA test followed by a Tukey's Multiple Comparison Test. P<0.05 was considerate as significant.

The Km of the ATP for JNK and the Km of biotin-cJun specific peptide were determined in the report AlphaScreen standardization assay

The mathematical relation between Ki and IC50 (Ki=IC50/(1+([Substrate]/Km of the substrate)) may be used to calculate the Ki values.

### Example 3

### Internalization Experiments and Analysis

### 3.1 Materials and Methods for Uptake Experiments

a) Cell Line:
The cell line used for this experiment was HL-60 (Ref CCL-240, ATCC, Lot 116523)

b) Culture Medium and Plates
RPMI (Ref 21875-091, Invitrogen, Lot 8296) or DMEM (Ref 41965, Invitrogen, Lot 13481) complemented on May 5, 2008 with:
   10% FBS (Ref A64906-0098, PAA, Lot A15-151): decomplemented at 56° C., 30 min, on Apr. 4, 2008.
   1 mM Sodium Pyruvate (Ref S8636, Sigma, Lot 56K2386)
   Penicillin (100 unit/ml)/Streptomycin (100 μg/ml) (Ref P4333, Sigma, Lot 106K2321)
PBS 10× (Ref 70011, Invitrogen, Lot 8277): diluted to 1× with sterile H₂O
Trypsine-0.05% EDTA (Ref L-11660, PAA, Lot L66007-1194)
6 well culture plates (Ref 140675, Nunc, Lot 102613)
24 well culture plates (Ref 142475, Nunc, Lot 095849)
96 well culture plates (Ref 167008, Nunc, Lot 083310)
96 well plates for protein dosing (Ref 82.1581, Sarstedt)
96 well plates for fluorescence measurement (Ref 6005279, Perkin Elmer)

c) Solutions
Poly-D-lysine coating solution (Sigma P9011 Lot 095K5104): 25 μg/ml final diluted in PBS 1×
Acidic wash buffer: 0.2M Glycin, 0.15M NaCl, pH 3.0
Ripa lysis buffer: 10 mM NaH₂PO₄ pH 7.2, 150 mM NaCl, 1% Triton X-100, 1 mM EDTA pH 8.0, 200 μM Na₃VO₂, 0.1% SDS, 1× protease inhibitor cocktail (Ref 11873580001, Roche, Lot 13732700)

d) Microscopy and Fluorescence Plate Reader
Cells were observed and counted using an inverted microscope (Axiovert 40 CFL; Zeiss; 20×).
The fluorescence was read with the Fusion Alpha Plate reader (Perkin Elmer).

e) Method
FITC marked peptide internalization was studied on suspension cells. Cells were plated into poly-DL-lysine coated dishes at a concentration of 1×10⁶ cells/ml. Plates were then incubated for 24 h at 37° C., 5% CO₂ and 100% relative humidity prior to the addition of a known concentration of peptide. After peptide addition, the cells were incubated 30 min, 1, 6 or 24 h at 37° C., 5% CO₂ and 100% relative humidity. Cells were then washed twice with an acidic buffer (Glycin 0.2 M, NaCl 0.15 M, pH 3.0) in order to remove the cell-surface adsorbed peptide (see Kameyama et al., (2007), Biopolymers, 88, 98-107). The acidic buffer was used as peptides rich in basic amino acids adsorb strongly on the cell surfaces, which often results in overestimation of internalized peptide. The cell wash using an acidic buffer was thus employed to remove the cell-surface adsorbed peptides. The acid wash was carried out in determining cellular uptake of Fab/cell-permeating peptide conjugates, followed by two PBS washes. Cells were broken by the addition of the RIPA lysis buffer. The relative amount of internalized peptide was then determined by fluorescence after background subtraction and protein content normalization.
The steps are thus: 1. Cell culture
   2. Acidic wash and cellular extracts
   3. Analysis of peptide internalization with a fluorescence plate reader

f) Cell Culture and Peptide Treatment

The 6 well culture plates are coated with 3 ml of Poly-D-Lys (Sigma P9011; 25 μg/ml in PBS), the 24 well plates with 600 μl and the 96 well plates with 125 μl and incubated for 4 h at 37° C., CO₂ 5% and 100% relative humidity.

After 4 hours the dishes were washed twice with 3.5 ml PBS, 700 μl or 150 μl PBS for the 6, 24 or 96 well plates, respectively.

The cells were plated into the dishes in 2.4 ml medium (RPMI) at plating densities of 1'000'000 cells/ml for suspension cells. After inoculation, the plates were incubated at 37° C., 5% CO₂ and 100% relative humidity for 24 hours prior to the addition of the peptide. Adherent cells should be at a density of 90-95% the day of treatment and were plated in DMEM:

| | Surface of | | | Nb | Nb |
|---|---|---|---|---|---|
| | culture | | | adherent | suspension |
| well | (cm²) | Medium | | cells | cells |
| 96 well | 0.3 | 100-200 | μl | 8′000- | 100′000 |
| | | | | 30′000 | |
| 24 well | 2 | 500-1000 | μl | 100′000- | 500′000- |
| | | | | 200′000 | 1′000′000 |
| 35 mm (P35)/ | 10 | 2, 4 | ml | 250′000- | 2′400′000 |
| 6 well | | | | 2′100′000 | |
| 60 mm (P60) | 20 | 3, 5 | ml | 15 * 10⁵ | 1′000′000/ml |
| 10 cm (P100) | 60 | 10 | ml | 15-60 * 10⁵ | |

The cells were treated with the desired concentration of FITC labeled peptide (stock solution at a concentration of 10 mM in H₂O).

Following peptide addition, the cells were incubated 0 to 24 hours (e.g. 30 min, 1, 6 or 24 hours) at 37° C., CO₂ 5% and 100% relative humidity.

Acidic Wash and Cellular Extracts:

The extracts were cooled on ice.

Suspension cells (or cells, which don attach well to the dish):

Transfer the cells in <<Falcon 15 ml>>. To recover the maximum of cells, wash the dish with 1 ml of PBS.

Harvest the cells 2 min at 2400 rpm max.

Suspend the cells in 1 ml cold PBS.

Transfer the cells into a coated “Eppendorf tube” (coated with 1 ml of poly D-Lys for 4 hours and washed twice with 1 ml PBS).

Wash three times with 1 ml of cold acidic wash buffer and centrifuge 2 min at 2400 rpm max.

Beware of the spreading of the cells in the “eppendorf”.

Wash twice with 1 ml cold PBS to neutralize.

Add 50 μl of lysis RIPA Buffer.

Incubate 30 min-1 h on ice with agitation.

Adherent cells:

Wash three times with 3 ml, 1 ml or 200 μl (for 6, 24 or 96 well plates, respectively) of cold acidic wash buffer. Beware of the cells who detach from the dish.

Wash twice with 1 ml cold PBS (for 6, 24 or 96 well plates, respectively) to neutralize.

Add 50 μl of lysis RIPA buffer.

Incubate 30 min-1 h on ice with agitation.

Scrap the cells with a cold scrapper. The 24 and 96 well plates were directly centrifuged at 4000 rpm at 4° for 15 min to remove the cellular debris. Then the supernatants (100 or 50 ml respectively for the 24 or 96 well plates) were directly transferred in a dark 96 well plated. The plates were read by a fluorescence plate reader (Fusion Alpha, Perkin Elmer).

Transfer the lysate in a coated “eppendorf” (coated with 1 ml of poly D-Lys for 4 hours and wash twice with 1 ml PBS).

The lysed cells were then centrifuged 30 min at 10000 g at 4° C. to remove the cellular debris.

Remove the supernatant and store it at −80° C. in a coated “Eppendorf tube” (coated with 1 ml of poly D-Lys for 4 hours and washed twice with 1 ml PBS).

Analysis of Peptide Internalization with a Fluorescence Plate Reader:

The content of each protein extract was determined by a standard BCA assay (Kit N^{o} 23225, Pierce), following the instructions of the manufacturer.

The relative fluorescence of each sample is determined after reading 10 μl of each sample in a fluorescence plate reader (Fusion Alpha, Perkin Elmer), background subtraction and normalization by protein concentration.

### 3.2 Uptake Experiments

The time dependent internalization (uptake) of FITC-labeled TAT derived transporter constructs into cells of the HL-60 cell line was carried out as described above using sequences transporter peptides of SEQ ID NOs: 52-96, 43, and 45-47. These sequences are listed below in Table 4.

TABLE 4
Transporter sequence tested in uptake experiments

| SEQ | peptide No: | | | | | | | | | | | |
|---|---|---|---|---|---|---|---|---|---|---|---|---|
| ID | abbreviation | | | | | | | | | | | |
| NO: | in FIG. 6 | | | | | | | | | | | |
| 46 | r3-L-TAT | H2N | dR | K | K | R | dR | Q | R | R | dR | CONH2 |
| 52 | 1 | H2N | dR | A | K | R | dR | Q | R | R | dR | CONH2 |
| 53 | 2 | H2N | dR | K | A | R | dR | Q | R | R | dR | CONH2 |
| 54 | 3 | H2N | dR | K | K | A | dR | Q | R | R | dR | CONH2 |
| 55 | 4 | H2N | dR | K | K | R | dR | A | R | R | dR | CONH2 |
| 56 | 5 | H2N | dR | K | K | R | dR | Q | A | R | dR | CONH2 |
| 57 | 6 | H2N | dR | K | K | R | dR | Q | R | A | dR | CONH2 |
| 58 | 7 | H2N | dR | D | K | R | dR | Q | R | R | dR | CONH2 |
| 59 | 8 | H2N | dR | K | D | R | dR | Q | R | R | dR | CONH2 |
| 60 | 9 | H2N | dR | K | K | D | dR | Q | R | R | dR | CONH2 |
| 61 | 10 | H2N | dR | K | K | R | dR | D | R | R | dR | CONH2 |
| 62 | 11 | H2N | dR | K | K | R | dR | Q | D | R | dR | CONH2 |
| 63 | 12 | H2N | dR | K | K | R | dR | Q | R | D | dR | CONH2 |
| 64 | 13 | H2N | dR | E | K | R | dR | Q | R | R | dR | CONH2 |
| 65 | 14 | H2N | dR | K | E | R | dR | Q | R | R | dR | CONH2 |
| 66 | 15 | H2N | dR | K | K | E | dR | Q | R | R | dR | CONH2 |
| 67 | 16 | H2N | dR | K | K | R | dR | E | R | R | dR | CONH2 |
| 68 | 17 | H2N | dR | K | K | R | dR | Q | E | R | dR | CONH2 |
| 69 | 18 | H2N | dR | K | K | R | dR | Q | R | E | dR | CONH2 |
| 70 | 19 | H2N | dR | F | K | R | dR | Q | R | R | dR | CONH2 |
| 71 | 20 | H2N | dR | K | F | R | dR | Q | R | R | dR | CONH2 |
| 72 | 21 | H2N | dR | K | K | F | dR | Q | R | R | dR | CONH2 |
| 73 | 22 | H2N | dR | K | K | R | dR | F | R | R | dR | CONH2 |
| 74 | 23 | H2N | dR | K | K | R | dR | Q | F | R | dR | CONH2 |
| 75 | 24 | H2N | dR | K | K | R | dR | Q | R | F | dR | CONH2 |
| 76 | 25 | H2N | dR | R | K | R | dR | Q | R | R | dR | CONH2 |
| 77 | 26 | H2N | dR | K | R | R | dR | Q | R | R | dR | CONH2 |
| 78 | 27 | H2N | dR | K | K | K | dR | Q | R | R | dR | CONH2 |
| 79 | 28 | H2N | dR | K | K | R | dR | R | R | R | dR | CONH2 |
| 80 | 29 | H2N | dR | K | K | R | dR | Q | K | R | dR | CONH2 |
| 81 | 30 | H2N | dR | K | K | R | dR | Q | R | K | dR | CONH2 |
| 82 | 31 | H2N | dR | H | K | R | dR | Q | R | R | dR | CONH2 |
| 83 | 32 | H2N | dR | K | H | R | dR | Q | R | R | dR | CONH2 |
| 84 | 33 | H2N | dR | K | K | H | dR | Q | R | R | dR | CONH2 |
| 85 | 34 | H2N | dR | K | K | R | dR | H | R | R | dR | CONH2 |
| 86 | 35 | H2N | dR | K | K | R | dR | Q | H | R | dR | CONH2 |
| 87 | 36 | H2N | dR | K | K | R | dR | Q | R | H | dR | CONH2 |
| 88 | 37 | H2N | dR | I | K | R | dR | Q | R | R | dR | CONH2 |
| 89 | 38 | H2N | dR | K | I | R | dR | Q | R | R | dR | CONH2 |
| 90 | 39 | H2N | dR | K | K | I | dR | Q | R | R | dR | CONH2 |
| 91 | 40 | H2N | dR | K | K | R | dR | I | R | R | dR | CONH2 |
| 92 | 41 | H2N | dR | K | K | R | dR | Q | I | R | dR | CONH2 |
| 93 | 42 | H2N | dR | K | K | R | dR | Q | R | I | dR | CONH2 |
| 94 | 43 | H2N | dR | L | K | R | dR | Q | R | R | dR | CONH2 |
| 45 | 44 (D-TAT) | H2N | dR | dR | dR | dQ | dR | dR | dK | dK | dR | CONH2 |
| 47 | 45 (r3-L-TATi) | H2N | dR | R | R | Q | dR | R | K | K | dR | CONH2 |
| 46 | 46 (r3-L-TAT) | H2N | dR | K | K | R | dR | Q | R | R | dR | CONH2 |
| 43 | 47 (L-TAT) | H2N | R | K | K | R | R | Q | R | R | R | CONH2 |
| 99 | 48 | H2N | dR | K | K | R | dR | Q | R | L | dR | CONH2 |
| 100 | 49 | H2N | dR | M | K | R | dR | Q | R | R | dR | CONH2 |
| 101 | 50 | H2N | dR | K | M | R | dR | Q | R | R | dR | CONH2 |
| 102 | 51 | H2N | dR | K | K | M | dR | Q | R | R | dR | CONH2 |
| 103 | 52 | H2N | dR | K | K | R | dR | M | R | R | dR | CONH2 |
| 104 | 53 | H2N | dR | K | K | R | dR | Q | M | R | dR | CONH2 |
| 105 | 54 | H2N | dR | K | K | R | dR | Q | R | M | dR | CONH2 |
| 106 | 55 | H2N | dR | N | K | R | dR | Q | R | R | dR | CONH2 |
| 107 | 56 | H2N | dR | K | N | R | dR | Q | R | R | dR | CONH2 |
| 108 | 57 | H2N | dR | K | K | N | dR | Q | R | R | dR | CONH2 |
| 109 | 58 | H2N | dR | K | K | R | dR | N | R | R | dR | CONH2 |
| 110 | 59 | H2N | dR | K | K | R | dR | Q | N | R | dR | CONH2 |
| 111 | 60 | H2N | dR | K | K | R | dR | Q | R | N | dR | CONH2 |
| 112 | 61 | H2N | dR | Q | K | R | dR | Q | R | R | dR | CONH2 |
| 113 | 62 | H2N | dR | K | Q | R | dR | Q | R | R | dR | CONH2 |
| 114 | 63 | H2N | dR | K | K | Q | dR | Q | R | R | dR | CONH2 |
| 115 | 64 | H2N | dR | K | K | R | dR | K | R | R | dR | CONH2 |
| 116 | 65 | H2N | dR | K | K | R | dR | Q | Q | R | dR | CONH2 |
| 117 | 66 | H2N | dR | K | K | R | dR | Q | R | Q | dR | CONH2 |
| 118 | 67 | H2N | dR | S | K | R | dR | Q | R | R | dR | CONH2 |
| 119 | 68 | H2N | dR | K | S | R | dR | Q | R | R | dR | CONH2 |
| 120 | 69 | H2N | dR | K | K | S | dR | Q | R | R | dR | CONH2 |
| 121 | 70 | H2N | dR | K | K | R | dR | S | R | R | dR | CONH2 |
| 122 | 71 | H2N | dR | K | K | R | dR | Q | S | R | dR | CONH2 |
| 123 | 72 | H2N | dR | K | K | R | dR | Q | R | S | dR | CONH2 |
| 124 | 73 | H2N | dR | T | K | R | dR | Q | R | R | dR | CONH2 |
| 125 | 74 | H2N | dR | K | T | R | dR | Q | R | R | dR | CONH2 |
| 126 | 75 | H2N | dR | K | K | T | dR | Q | R | R | dR | CONH2 |
| 127 | 76 | H2N | dR | K | K | R | dR | T | R | R | dR | CONH2 |
| 128 | 77 | H2N | dR | K | K | R | dR | Q | T | R | dR | CONH2 |
| 129 | 78 | H2N | dR | K | K | R | dR | Q | R | T | dR | CONH2 |
| 130 | 79 | H2N | dR | V | K | R | dR | Q | R | R | dR | CONH2 |
| 131 | 80 | H2N | dR | K | V | R | dR | Q | R | R | dR | CONH2 |
| 132 | 81 | H2N | dR | K | K | V | dR | Q | R | R | dR | CONH2 |
| 133 | 82 | H2N | dR | K | K | R | dR | V | R | R | dR | CONH2 |
| 134 | 83 | H2N | dR | K | K | R | dR | Q | V | R | dR | CONH2 |
| 135 | 84 | H2N | dR | K | K | R | dR | Q | R | V | dR | CONH2 |
| 136 | 85 | H2N | dR | W | K | R | dR | Q | R | R | dR | CONH2 |
| 137 | 86 | H2N | dR | K | W | R | dR | Q | R | R | dR | CONH2 |
| 138 | 87 | H2N | dR | K | K | W | dR | Q | R | R | dR | CONH2 |
| 139 | 88 | H2N | dR | K | K | R | dR | W | R | R | dR | CONH2 |
| 140 | 89 | H2N | dR | K | K | R | dR | Q | W | R | dR | CONH2 |
| 141 | 90 | H2N | dR | K | K | R | dR | Q | R | W | dR | CONH2 |
| 142 | 91 | H2N | dR | Y | K | R | dR | Q | R | R | dR | CONH2 |
| 143 | 92 | H2N | dR | K | Y | R | dR | Q | R | R | dR | CONH2 |
| 144 | 93 | H2N | dR | K | K | Y | dR | Q | R | R | dR | CONH2 |
| 145 | 94 | H2N | dR | K | K | R | dR | Y | R | R | dR | CONH2 |
| 146 | 95 | H2N | dR | K | K | R | dR | Q | Y | R | dR | CONH2 |
| 147 | 96 | H2N | dR | K | K | R | dR | Q | R | Y | dR | CONH2 |

In the above table D amino acids are indicated by a small “d” prior to the respective amino acid residue (e.g. dR=D-Arg).

For a few sequences synthesis failed in the first approach due to technical reasons. These sequences are abbreviated in FIG. 6 as 1, 2, 3, 4, 5, 6, 7, 8, 43, 52, 53, 54, 55, 56, 57, 85, 86, 87, 88, 89, and 90. All the remaining sequences were used in the internalization experiments.

The results are shown in FIG. 6.

As can be seen in FIG. 6, after 24 hours of incubation, all transporters with the consensus sequence rXXXrXXXr (SEQ ID NO: 31) showed a higher internalization capability than the L-TAT transporter (SEQ ID NO: 43). Hela cells were incubated 24 hours in 96 well plate with 10 mM of the r3-L-TAT-derived transporters. The cells were then washed twice with an acidic buffer (0.2M Glycin, 0.15M NaCl, pH 3.0) and twice with PBS. Cells were broken by the addition of RIPA lysis buffer. The relative amount of internalized peptide was then determined by reading the fluorescence intensity (Fusion Alpha plate reader; PerkinElmer) of each extract followed by background subtraction

As can be seen in FIG. 6, one position appears to be critical for highest transporter activity and for improved kinetics of transport activity: Y in position 2 (peptide N^{o} 91 corresponding to SEQ ID NO: 142).

The conclusion from the results of this experiment is as follows:
After 24 hours incubation, all transporters with the consensus sequence rXXXrXXXr (SEQ ID NO: 31) (see Table 2 for a selection of possible sequences) showed a higher internalization capability than the L-TAT transporter (SEQ ID NO: 43) (FIG. 6). Those results fully validate the consensus sequence rXXXrXXXr (SEQ ID NO: 31).
One position is critical for highest transporter activity and (FIG. 6): Y in position 2 (sequence 91 corresponding to SEQ ID NO: 142).

Accordingly, such TAT derived sequences as shown in Table 4 are preferred, which exhibit an Y in position 2, particularly when the sequence exhibits 9 aa and has the consensus sequence rXXXrXXXr (SEQ ID NO: 31).

### Example 4

### Measurement of Cytokine and Chemokine Release

In the following the procedure will be set forth describing how the released amount of several human cytokines after ligand induced secretion from human cells (Blood, WBC, PBMC, purified primary lymphocytes, cell lines, . . . ) was measured.

The technique used is a Sandwich ELISA, which allows measuring the amount of antigen between two layers of antibodies (i.e. capture and detection antibody). The antigen to be measured must contain at least two antigenic sites capable of binding to antibody, since at least two antibodies act in the sandwich. Either monoclonal or polyclonal antibodies can be used as the capture and detection antibodies in Sandwich ELISA systems. Monoclonal antibodies recognize a single epitope that allows fine detection and quantification of small differences in antigen. A polyclonal is often used as the capture antibody to pull down as much of the antigen as possible. The advantage of Sandwich ELISA is that the sample does not have to be purified before analysis, and the assay can be very sensitive (up to 2 to 5 times more sensitive than direct or indirect).

The method may be used to determine the effect of the JNK inhibitors of the present invention in vitro/cell culture. At non toxic doses, compound efficacy is indicated by the decrease of the cytokine levels (the variation of optical density (absorbance at 450 nm)) as compared to non-treated samples and is monitored by ELISA. Results are express in ng/ml.

### 4.1 Material

96 well plate:
   for collecting the supernatants (Ref 82.1581, Sarstedt)
   for ELISA (F96 maxisorp, Ref 442404, Nunc)
TopSeal-A: 96 well microplate seals (Ref 600585, PerkinElmer).
ELISA reagent
   Coating buffer ELISA: 0.1M NaCarbonate pH 9.5 (=7.13 g NaHCO₃ (ref 71627, Fluka)+1.59 g Na₂CO₃ (ref 71345, Fluka) in 1 litre H2O, pH to 9.5 with NaOH concentrated)
   Wash buffer ELISA: PBS 1×+0.01% Tween20. Prepare 1 litre PBS 1× (PBS10×: ref 70011, GIBCO) and add 100 ul of Tween20 (ref P1379, Sigma) slowly while mixing with magnetic agitator)
   Assay diluent: PBS 1×+10% FBS (Ref A15-151, PAA, decomplemented at 56° C., 30 min).
   DAKO TMB (ref S1599, DAKO): commercial substrate solution
   Stop Solution: 1M H₃PO₄ (4 for 200 ml=177 ml H₂O+23 ml H₃PO₄ 85% (ref 345245, Aldrich).
ELISA Kit (reagent for 20 plates)
   IFN-γ: Human IFN-ELISA set, BD OptEIA™ (ref 555142, DB).
   IL-1β: Human IL-1 ELISA set II, BD OptEIA™ (ref 557953, BD)
   IL-10: Human IL-10 ELISA set II, BD OptEIA™ (ref 555157, DB).
   IL-12: Human IL-12 (p70) ELISA set, BD OptEIA™ (ref 555183, DB).
   IL-15: Human IL-15 ELISA Set, BD OptEIA™ (ref 559268, DB).
   IL-2: Human IL-2 ELISA set, BD OptEIA™ (ref 555190, DB).
   IL-4: Human IL-4 ELISA set, BD OptEIA™ (ref 555194, DB).
   IL-5: Human IL-5 ELISA set, BD OptEIA™ (ref 555202, DB).
   IL-6: Human IL-6 ELISA set I, BD OptEIA™ (ref 555220, DB).
   IL-8: Human IL-8 ELISA set, BD OptEIA™ (ref 555244, DB).
   MCP-1: Human MCP-1 ELISA set, BD OptEIA™ (ref 555179, BD)
   TNF-α: Kit human TNF ELISA set, BD OptEIA™ (ref 555212, DB).
Absorbance reading: The absorbance was read on the Fusion Alpha Plate reader (Perkin Elmer).
Repeating pipettes, digital pipettes or multichannel pipettes.

### 4.2 Method

Preparation of the Samples
The samples are culture medium supernatant from cultured human cells (typically whole blood, WBC, PBMC, Purified subtype of WBC, cancerous cell lines). Remove any particulate material by centrifugation (400 g 5 min 4° C.) and assay immediately or store samples at −20° C. Avoid repeated freeze-thaw cycles.
One hour before using, defrost the samples on ice and centrifuge them. At step 11, dilute the samples in assay diluent directly into the plate (add first assay diluent, then the samples and pipette up and down):

Preparation of Standard
After warming lyophilized standard to room temperature, carefully open vial to avoid loss of material. Reconstitute lyophilized standard with the proposed volume of deionized water to yield a stock standard. Allow the standard to equilibrate for at least 15 minutes before making dilutions. Vortex gently to mix. After reconstitution, immediately aliquot standard stock in polypropylene vials at 50 μl per vial and freeze at −20° C. for up to 6 months. If necessary, store at 2-8° C. for up to 8 hours prior to aliquotting/freezing. Do not leave reconstituted standard at room temperature.
Immediately before use, prepare a ten point standard curve using 2-fold serial dilutions in reagent Diluent. A high standard of 4000 μg/ml is recommended.

Preparation of Detector Mix
One-step incubation of Biotin/SAv reagents. Add required volume of Detection Antibody to Assay Diluent. Within 15 minutes prior to use, add required quantity of Enzyme Reagent, vortex or mix well. For recommended dilutions, see lot-specific Instruction/Analysis Certificate. Discard any remaining Working Detector after use.

Coating with Capture Antibody
1. Coat the wells of a PVC microtiter plate with 100 μL per well of Capture Antibody diluted in Coating Buffer. For recommended antibody coating dilution, see lot-specific Instruction/Analysis Certificate.
2. Cover the plate with an adhesive plastic and incubate overnight at 4° C.
3. Remove the coating solution and wash the plate by filling the wells with 150 μl wash buffer.
4. The solutions or washes are removed by flicking the plate over a sink.
5. Repeat the process two times for a total of three washes.
6. After the last wash, remove any remaining wash buffer by patting the plate on a paper towel.

Blocking
7. Block the remaining protein-binding sites in the coated wells by adding 100 μl reagent Diluent per well.
8. Cover the plate with an adhesive plastic and incubate for 1 h at room temperature.
9. During the incubation, start preparing the standard.

Adding Samples
10. Do one wash as in step 3 with 150 μl of wash buffer. The plates are now ready for sample addition.
11. Add 50 μl of appropriately diluted samples in assay diluent to each well. For accurate quantitative results, always compare signal of unknown samples against those of a standard curve. Standards (triplicates) and blank must be run with each cytokine to ensure accuracy.
12. Cover the plate with an adhesive plastic and incubate for 2 h at room temperature.

Incubation with Detection Antibody and Secondary Antibody
13. Wash the plate four times with 150 μl wash buffer like step 3.
14. Add 50 μl of detector MIX (detection antibody+Secondary Streptavidin-HRP antibody in assay diluent) to each well at recommended dilutions (see lot-specific Instruction/Analysis Certificate).
15. Cover the plate with an adhesive plastic and incubate for 1 h at room temperature light protect.
16. Wash the plate six times with 150 μl wash buffer as in step 3.
17. Add 50 μl DAKO TMB solution to each well, incubate for 15-20 min at room temperature, in the dark, not sealed.
18. Add 50 μl of stop solution to each well. Gently tap the plate to ensure thorough mixing.
19. Mix the plate 5 min at 500 rpm on a plate mixer.
20. Read the optical density at 450 nm. (Program: Cytokine_ELISA on Fusion Alpha Plate reader).

### Data Analysis

Average the triplicate readings for each standard control and each sample. Subtract the average zero standard optical density (O.D). Create a standard curve plotting the log of the cytokine concentration versus the log of the O.D and the best fit line can be determined by regression analysis. If samples have been diluted, the concentration read from the standard curve must be multiplied by the dilution factor. A standard curve should be generated for each set of samples assayed. The outliers data were avoided using Grugg's test. Then the data which weren't in the interval of two times the SD, were discard. The independent experiments are taken into account if the positive control showed data as previously observed. The independent experiments are pooled (N>3).

The data are presented in μg/ml of cytokine release or in %, compared to the induced condition without inhibitor treatment.

### Example 5

### THP1 Differentiation—Stimulation for Cytokine Release

In the following the procedure will be set forth describing how cytokine production from human PMA differentiated THP1 cells challenged by LPS for 6 h was induced in order to test the ability of JNK inhibitors of the present invention, in particular of a JNK inhibitor with SEQ ID NO: 172, to reduce stimulation-induced cytokine release. THP1 cells were stimulated ex-vivo by different ligands for the readout of cytokine release. At non toxic doses, JNK inhibitor efficacy is indicated by the decrease of the cytokine levels as compared to non-treated samples and is monitored by ELISA. The toxicity of the compound are evaluated by the reduction of a tretazolium salt (MTS) to formazan, giving a purple colour.

### Procedure:

a. Material
Cell Line: THP-1 (Ref TIB-202, ATCC, lot 57731475)
Culture medium, reagent and plates
RPMI (Ref 21875-091, Invitrogen) complemented with:
10% FBS (Ref A15-151, PAA): decomplemented at 56° C., 30 min.
10 mM Hepes (Ref H0887, Sigma)
50 M-mercaptoethanol (Ref 63690, Fluka: stock at 14.3M): add 5601 of 50 mM aliquots in PBS stocked at −20° C.)
1 mM Sodium Pyruvate (Ref S8636, Sigma)
Penicilline (100 unit/ml)/Streptomycine (100 g/ml) (Ref P4333, Sigma)
The RPMI medium is then filtrated with a 0.22 M filter (Ref SCGPU05RE, Millipore).
PBS 10× (Ref 70011, Invitrogen): diluted to 1× with sterile H₂O
DMSO: Ref 41444, Fluka
PMA (phorbol 12-myristate β-acetate, Ref P1585, Sigma, concentration 1 mM=616.8 ug/ml in DMSO at −20° C.). Use directly at a final concentration of 100 nM in RPMI (1 ul in 10 ml of medium).
LPS ultrapure (Lipopolysaccharide, Ref tlrl-eklps, Invivogen, concentration 5 mg/ml): Stock solution of LPS: 3 g/ml in PBS at 4° C. Use directly to prepare a 4× concentrated solution of 40 ng/ml in RPMI medium (min 1800 l/plate; for 5 plates: 125 l of LPS 3 g/ml+9250 l RPMI).
96 well plate:
for adherent cell culture (Ref 167008, Nunc)
for collecting the supernatants (Ref 82.1581, Sarstedt)
for ELISA (F96 maxisorp, Ref 442404, Nunc)
Coating solutions: poly-D-lysine (Ref P9011, Sigma): 25 g/ml final diluted in PBS 1×
ELISA reagent and kits
Coating buffer ELISA: 0.1 M NaCarbonate pH 9.5 (=7.13 g NaHCO₃ (ref 71627, Fluka)+1.59 g Na₂CO₃ (ref 71345, Fluka) in 1 liter H2O, pH to 9.5 with NaOH concentrated)
Wash buffer ELISA: PBS 1×+0.01% Tween20 (ref P1379, Sigma, lot 094K0052)(=prepare 1 liter PBS 1× and add 100 ul of Tween20 slowly while mixing with magnetic agitator)
Assay diluent: PBS 1×+10% FBS (Ref A15-151, PAA, decomplemented at 56° C., 30 min).
DAKO TMB (ref S1599, DAKO): commercial substrate solution
Stop Solution: 1M H₃PO₄ (→for 200 ml=177 ml H₂O+23 ml H₃PO₄ 85% (ref 345245, Aldrich).
TNF-: Kit human TNF ELISA set, BD OptEIA (ref 555212, DB).
Cytotoxicity measurement: CellTiter 96 reagent (ref G3581, Promega)
Control compound: SP600125 (ref ALX-270-339-M025, Alexis, concentration: 20 mM DMSO)
Absorbance reading: The absorbance was read on the Fusion Alpha Plate reader (Perkin Elmer).
Repeating pipettes, digital pipettes or multichannel pipettes.
TopSeal-A: 96 well microplate seals (Ref 600585, PerkinElmer).
   b. Method

### Well Coating

The plates had been coated with 200 l of poly D-Lysine (1×) and incubated 2 hours at 37° C., CO₂ 5% and 100% relative humidity.

### Cell Plating

After 2 hours the wells were washed twice with 200 l PBS 1× (use immediately or leave with 200 l of PBS 1× at 37° C. till use, but no more than 3 days).

The cells were counted. The desired number of cells was taken and resuspended in the amount of media necessary to get a dilution of 1'000'000 cells/ml. 100 nM of PMA was added to induce the differentiation of the THP1 from suspension monocytes to adherent macrophages. The cells were plated into the wells in 100 l medium at plating densities of 100'000 cells/well. After inoculation, the plates were incubated at 37° C., 5% CO2 and 100% relative humidity 3 days to let them differentiate, prior to the addition of experimental drugs.

### Cell Treatment

After 3 days, the adherent cells were observed with the microscope. The media containing PMA was aspirated and replaced by 100 l of fresh RPMI media without PMA (no washing step with PBS 1×).

Experimental drug were prepared at the concentration of 10 mM in H₂O or DMSO and stored at −80° C. Prior to each daily use, one aliquot of JNK inhibitor was defrost and diluted to reach a 4× concentrated solution (120 M) in RPMI medium and then to the desired concentration in RPMI. The SP600125 was diluted to reach a 4× concentrated solution (40 M) in RPMI medium and then to the desired concentration in RPMI containing 0.8% DMSO.

The plates were treated with 50 l of medium or a solution of 4× the final desired drug concentration (0, 100 nM, 1, 3, 10 or 30 M final for JNK compound or at 0, 10, 100 nM, 1, 3 or 10 M final for the SP600125 positive control). Following drug addition, the plates were incubated for an additional 1 h at 37° C., 5% CO₂ and 100% relative humidity.

After 1 hours, the secretion of TNF was induced by the addition of 50 l of a 4× concentrated dilution of LPS ultrapure (3 ng/ml final).

### Assay

After 6 hours, 100 l of the supernatant were transferred to new 96 well plates. Those plates were sealed and stored at −20° till the analysis by ELISA (e.g. see example 4) of the secretion of the cytokines.

The cytotoxic effect of the compounds was evaluated by MTS absorbance (e.g. see example 4) and cells were observed using an inverted microscope (Axiovert 40 CFL; Zeiss; 10×).

### Data Analysis

Analyses of the data are performed as indicated in the ELISA (see example 4). Briefly, for ELISA: Average the triplicate readings for each standard control and each sample. Subtract the average zero standard optical density (O.D). Create a standard curve plotting the log of the cytokine concentration versus the log of the O.D and the best fit line can be determined by regression analysis. If samples have been diluted, the concentration read from the standard curve must be multiplied by the dilution factor. A standard curve should be generated for each set of samples assayed. The outliers data were avoid using Grugg's test. Then the data which weren't in the interval of two times the SD, were discard. The independent experiments are taken into account if the positive control showed data as previously observed. The independent experiments are pooled (N>3).

For the Cytotoxicity effect evaluation: on each plate of each independent experiment taken into account for the cytokine release experiment analysis, the average of the absorbance of the medium alone was considerate as the background and subtracted to each absorbance value. The average of triplicate of the non treated cells of each compound was considerate as the 100% viability. The average of triplicate of each compound was normalized by its 100%. The outliers data were avoid using Grugg's test. Then the data which weren't in the interval of two times the SD, were discard. The independent experiments are pooled (N>3).

All statistical comparisons of conditions were performed by the GraphPad Prism4 software with the following test: One way ANOVA test followed by a Tukey's Multiple Comparison Test. P<0.05 was considerate as significant.

### Example 6

### JNK Inhibitor of SEQ ID NO: 172 and TNFα Release in Primary Rat or Human Whole Blood Cells

Whole blood is collected from anesthetized rat or human healthy volunteers using a venipuncture connected to a pre-labeled vacuum tube containing sodium citrate. Tubes are gently mixed by inversion 7-8 times; and are then kept at RT until stimulation. JNK inhibitor of SEQ ID NO: 172 is prepared 6 times concentrated in PBS, and 30 μl/well of mix is added into 96-well plate. Whole blood is diluted by 1:2 in PBS and 120 μl of diluted blood is added in each well where either PBS alone or JNK inhibitor of SEQ ID NO: 172 has been previously added. Whole blood is incubated at 37° C.; 85 rpm (Stuart Orbital incubator SI500) for 60 min. Activators (LPS) are the prepared, 30 μl/well of LPS, 6 times concentrated. After 60 min incubation, LPS is added to the blood, blood is mixed by pipetting up and down, and then kept for 4 h under agitation (85 rpm), at 37° C. After the 4 h incubation, the plates are centrifuged at about 770 g, 4° C. for 15 min in a pre-cooled centrifuge. Supernatants are finally collected and kept at −20° C. until cytokine measurement. Cytokine (IL-6, IL-2, IFNγ and TNFα) were then measured using standard Elisa kits (e.g. from R&D Systems: DuoSet Elisas; or from BD Biosciences: BD Opteia Set Elisa). Results are expressed as pg/ml of supernatant of the measured cytokine.

A similar experiment was conducted with PMA+ionomycin instead of LPS as activator/stimulant.

### Example 7

### Half-Life of Specific JNK Inhibitors Disclosed Herein

The JNK inhibitors with the sequence of SEQ ID NOs: 196, 197, and 172 (0.1 mM final concentration) were digested in human serum (10 and 50% in PBS 1×). The experiment was performed as described by Tugyi et al. (Proc Natl Acad Sci USA, 2005, 413-418). The remaining intact peptide was quantified by UPLC-MS. Stability was assessed for SEQ ID NOs: 196, 197, and 172 identically but in two separate assays. While the JNK inhibitor with SEQ ID NO: 196 was totally degraded into amino acids residues within 6 hours, the JNK inhibitor with SEQ ID NO: 172 was completely degraded only after 14 days. The JNK inhibitor with SEQ ID NO: 197 was still stable after 30 days.

### Example 8

### Dose-Dependent Inhibition by JNK Inhibitor with Sequence of SEQ ID NO: 172 of CD3/CD28-Induced IL-2 Release in Rat Primary T-Cells

Control animal were sacrificed, lymph nodes (LN) were harvested and kept in complete RPMI medium. LN were smashed with complete RPMI on 70 μm filter using a 5 ml piston. A few drops of media were added to keep strainer wet. Cells were centrifuged for 7 min at 450 g and 4° C. Pellet was resuspended in 5 ml fresh medium. Cells were passed again through cell strainer. An aliquot of cells was counted, while cells were centrifuged again 10 min at 1400 rpm and 4° C. Cells were resupended in MACS buffer (80 μl of MACS buffer per 10⁷ cells). 10 μl of anti-rat MHC microbeads were added per 10 million cells, cells were incubated for 15 min at 4°-8° C. Cells were washed with 15 ml MACS buffer and centrifuge for 7 min at 700 g and 4° C. Pellet was resuspended in 500 μl MACS buffer per 10⁸ cells. One LS column was placed in the magnetic field of the MACS separator per animal. Column was first rinsed with 3 ml of MACS buffer. One tube was placed below the column in ice to collect cells=T cells (negative selection so we collect what is eluted). Cell suspension was added and elute was collected on ice. Column was washed 3 times with 3 mL MACS buffer. Eluted T cells were centrifuges for 7 min at 700 g and 4° C. Resuspended cells were counted and plated at density of 200000 cells/well in 100 μl of complete medium. Plates were pre-coated the day before experiment with 2 μg/mL of CD3 antibody, and the day of experiment plates were washed three times with PBS. Cells were treated with 100 μl of (poly-)peptide JNK inhibitor (SEQ ID NO: 172), two times concentrated for 1 h before ligand activation. After 1 h of pre-treatment with (poly-)peptide JNK inhibitor (SEQ ID NO: 172), cells were then stimulated with 2 μg/mL of anti CD28 antibody for 24 h. After 24 h of stimulation, supernatant were collected and stored at −20° C. until analysis. Cytokines were then measured using standard Elisa kits. Results are expressed as pg/ml of supernatant of the measured cytokine.

In a further experiment, essentially the same protocol as set forth above was used, but in addition to the (poly-)peptide JNK inhibitors with SEQ ID NO: 172, JNK inhibitors with the sequence of SEQ ID NO: 197 and the drug molecule SP600125 were also tested thus allowing to compare the effects of these inhibitors on the inhibition of CD3/CD28-induced IL-2 release.

### Example 9

### JNK Inhibitor and TNFα/IL-2 Release in Human Whole Blood

Whole blood from human healthy volunteers was collected using a venipuncture connected to a pre-labeled vacuum tube containing sodium citrate. Tubes are gently mixed by inversion 7-8 times; and are then kept at RT until stimulation. 350 μl of RPMI+P/S were added into 1.2 ml-96-well plate. 10 times concentrated of SEQ ID NO: 172 was prepared in RPMI+P/S (50 μl per well). 50 μl was added into 1.2 ml-96 well plates. 50 μl of whole blood was then added in each well where either medium alone or JNK inhibitor has been previously added. Whole blood was incubated at 37° C., 5% CO2 for 60 min. 50 μl/well of ligands diluted in RPMI+P/S was prepared, corresponding to the final dilution 10 times concentrated. After 60 min of incubation, ligand was added; wells were then mixed by pipetting up and down the blood. Whole blood was incubated for 3 days at 37° C. (wells were mixed by pipetting each well up and down once per day). At the end of incubation, plates were mixed and then centrifuged at 2500 rpm, 4° C. for 15 min in a pre-cooled centrifuge. Cytokine were then measured using standard Elisa kits. Results are expressed as pg/ml of supernatant of the measured cytokine.

A similar experiment was carried out with slight modifications. In the case of CD3/CD8 stimulation, CD3 antibody was coated at 2 μg/mL in PBS overnight at 4° C. The day of experiment, wells were washed three times with PBS and left in PBS until use at 37° C. CD28 antibody was added 1 h after SEQ ID NO: 172 at final concentration of 2 μg/mL; supernatants were collected after 3 days of stimulation.

### Example 10

### Anti-Inflammatory Potency in a Rat Model of Endotoxins Induced Uveitis (EIU)

The anti-inflammatory potency of the JNK inhibitor of SEQ ID NO: 172 was tested in albino rats following intravenous administration (EIU/LPS model). The aim of this study was to determine the effects of single intravenous injections of SEQ ID NO: 172 (0.015, 0.18, and 1.80 mg/kg) on the inflammatory response in an endotoxins-induced uveitis albino rat model and to compare these affects to those obtained with prior art JNK inhibitor of SEQ ID NO: 197 (2 mg/kg). As a further control served phosphate sodic dexamethasone.

Sixty (60) male Lewis rats were randomly divided into six (6) groups of ten (10) animals each. EIU was induced by footpad injection of lipopolysaccharide (LPS, 1 mg/kg). NaCl (0.9%), SEQ ID NO: 197 at 2 mg/kg and SEQ ID NO: 172 at three concentrations (1.80 mg/kg, 0.18 mg/kg and 0.015 mg/kg) were administered by intravenous injection. Phosphate sodic dexamethasone (20 μg/eye) was administered by sub-conjunctival injection in both eyes. 24 hours after LPS injection, inflammatory response was evaluated by clinical scoring.

The intensity of clinical ocular inflammation was scored on a scale from 0 to 4 for each eye:

| | |
|---|---|
| Grade 0 | no inflammation |
| Grade 1 | slight iris and conjunctival vasodilation |
| Grade 2 | moderate iris and conjunctival vasodilation with flare |
| Grade 3 | intense iris and conjunctival vasodilation with flare |
| Grade 4 | intense inflammatory reaction |

(+1) fibrin formation and seclusion of pupils

Twenty-four hours after LPS induction, clinical scores for the vehicle-treated rats were 3.6±0.2 (mean±SEM, n=20) with a median of 4 (range, 2-5). A significant reduction (p<0.001) in the severity of the ocular inflammation was detected 24 hours after induction and intravenous treatment with SEQ ID NO: 197 (2 mg/kg) (mean score: 2.2±0.3, median: 2), corresponding to a 40% decrease of EIU scores compared with the score observed in vehicle group. Intravenous treatment with SEQ ID NO: 172, at approximately the same dose (1.80 mg/kg) reduced also significantly the severity of the ocular inflammation by 42% (mean score: 2.1±0.3, median: 2, p=0.001). The lower doses (0.18 and 0.015 mg/kg) reduced by 33% (mean score: 2.4±0.3, median: 2) and 36% (mean score: 2.3±0.3, median: 2) the inflammation, respectively. The reduction was significant with p<0.001.

A sub-conjunctival treatment with dexamethasone (20 μg/eye), used as positive control drug also significantly reduced the clinical scores by 79% (mean score: 0.8±0.2, median: 0.5, p<0.001).

Under these experimental conditions, it can be stated that a single intravenous injection of SEQ ID NO: 197 at 2 mg/kg partially prevented the endotoxin-induced inflammation observed in the anterior chamber. In comparison, SEQ ID NO: 172 intravenously injected at 0.015, 0.18, 1.80 mg/kg also reduced the endotoxin-induced inflammation in the anterior chamber.

### Example 11

### Dose-Responsive Effects after Intravenous Administration of JNK Inhibitor after 14 Days in a Rat Model of Chronic Established Type II Collagen Arthritis

Rat collagen arthritis is an experimental model of polyarthritis that has been widely used for preclinical testing of numerous anti-arthritic agents that are either under preclinical or clinical investigation or are currently used as therapeutics in this disease. The hallmarks of this model are reliable onset and progression of robust, easily measurable polyarticular inflammation, marked cartilage destruction in association with pannus formation, and mild to moderate bone resorption and periosteal bone proliferation.

Intravenous (IV) efficacy of the JNK inhibitor of SEQ ID NO: 172 administered daily (QD) for 14 days (arthritis d1-14) for inhibition of the inflammation (paw swelling), cartilage destruction, and bone resorption that occurs in established type II collagen arthritis in rats was determined in said experimental model.

Animals (8/group for arthritis) were anesthetized with Isoflurane and injected with 300 μl of Freund's Incomplete Adjuvant (Difco, Detroit, Mich.) containing 2 mg/ml bovine type II collagen (Elastin Products, Owensville, Mo.) at the base of the tail and 2 sites on the back on days 0 and 6. On day 10 of the study (arthritis d0), onset of arthritis occurred and rats were randomized into treatment groups. Randomization into each group was done after ankle joint swelling was obviously established in at least one hind paw.

Female Lewis rats with established type II collagen arthritis were treated daily (QD) on arthritis days 1-14 by the intravenous (IV) route with vehicle (NaCl), SEQ ID NO: 172 (0.01, 0.1, 1, or 5 mg/kg), or the reference compound dexamethasone (Dex, 0.05 mg/kg). Animals were terminated on arthritis day 14. Efficacy evaluation was based on animal body weights, daily ankle caliper measurements, ankle diameter expressed as area under the curve (AUC), terminal hind paw weights, and histopathologic evaluation of ankles and knees of selected groups.

Scoring of Joints Collagen arthritic ankles and knees are given scores of 0-5 for inflammation, pannus formation and bone resorption according to the following criteria:

Knee and/or Ankle Inflammation

| | |
|---|---|
| 0 | Normal |
| 0.5 | Minimal focal inflammation |
| 1 | Minimal infiltration of inflammatory cells in synovium/periarticular |
| | tissue |
| 2 | Mild infiltration |
| 3 | Moderate infiltration with moderate edema |
| 4 | Marked infiltration with marked edema |
| 5 | Severe infiltration with severe edema |

### Ankle Pannus

| | |
|---|---|
| 0 | Normal |
| 0.5 | Minimal infiltration of pannus in cartilage and subchondral bone, |
| | affects only marginal zones and affects only a few joints |
| 1 | Minimal infiltration of pannus in cartilage and subchondral bone, |
| | primarily affects marginal zones |
| 2 | Mild infiltration (<¼ of tibia or tarsals at marginal zones) |
| 3 | Moderate infiltration (¼ to ⅓ of tibia or small tarsals affected at |
| | marginal zones) |
| 4 | Marked infiltration (½ to ¾ of tibia or tarsals affected at marginal |
| | zones) |
| 5 | Severe infiltration (>¾ of tibia or tarsals affected at marginal zones, |
| | severe distortion of overall architecture) |

### Knee Pannus

| | |
|---|---|
| 0 | Normal |
| 0.5 | Minimal infiltration of pannus in cartilage and subchondral bone, |
| | affects only marginal zones and affects only a few joints |
| 1 | Minimal infiltration of pannus in cartilage and subchondral bone, |
| | approximately 1-10% of cartilage surface or subchondral bone |
| | affected |
| 2 | Mild infiltration (extends over up to¼ of surface or subchondral |
| | area of tibia or femur), approximately 11-25% of cartilage surface |
| | or subchondral bone affected |
| 3 | Moderate infiltration (extends over >¼ but <½ of surface or |
| | subchondral area of tibia or femur) approximately 26-50% of |
| | cartilage surface or subchondral bone affected |
| 4 | Marked infiltration (extends over ½ to ¾ of tibial or femoral |
| | surface) approximately 51-75% of cartilage surface or subchondral |
| | bone affected |
| 5 | Severe infiltration approximately 76-100% of cartilage surface or |
| | subchondral bone affected |

### Ankle Cartilage Damage (Emphasis on Small Tarsals)

| | |
|---|---|
| 0 | Normal |
| 0.5 | Minimal decrease in T blue staining, affects only marginal zones and |
| | affects only a few joints |
| 1 | Minimal = minimal to mild loss of toluidine blue staining with no |
| | obvious chondrocyte loss or collagen disruption |
| 2 | Mild = mild loss of toluidine blue staining with focal mild |
| | (superficial) chondrocyte loss and/or collagen disruption |
| 3 | Moderate = moderate loss of toluidine blue staining with |
| | multifocal moderate (depth to middle zone) chondrocyte loss and/or |
| | collagen disruption, smaller tarsals affected to ½ to ¾ depth |
| | with rare areas of full thickness loss |
| 4 | Marked = marked loss of toluidine blue staining with multifocal |
| | marked (depth to deep zone) chondrocyte loss and/or collagen |
| | disruption, 1 or 2 small tarsals surfaces have full thickness loss of |
| | cartilage |
| 5 | Severe = severe diffuse loss of toluidine blue staining with |
| | multifocal severe (depth to tide mark) chondrocyte loss and/or |
| | collagen disruption affecting more than 2 cartilage surfaces |

### Knee Cartilage Damage

| | |
|---|---|
| 0 | Normal |
| 0.5 | Minimal decrease in T blue staining, affects only marginal zones |
| 1 | Minimal = minimal to mild loss of toluidine blue staining with |
| | no obvious chondrocyte loss or collagen disruption |
| 2 | Mild = mild loss of toluidine blue staining with focal mild |
| | (superficial) chondrocyte loss and/or collagen disruption, may have |
| | few small areas of 50% depth of cartilage affected |
| 3 | Moderate = moderate loss of toluidine blue staining with |
| | multifocal to diffuse moderate (depth to middle zone) |
| | chondrocyte loss and/or collagen disruption, may have 1-2 small |
| | areas of full thickness loss affecting less than ¼ of the total |
| | width of a surface and not more than 25% of the total width of all |
| | surfaces |
| 4 | Marked = marked loss of toluidine blue staining with multifocal |
| | to diffuse marked (depth to deep zone) chondrocyte loss and/or |
| | collagen disruption or 1 surface with near total loss and partial |
| | loss on others, total overall loss less than 50% of width of all |
| | surfaces combined |
| 5 | Severe = severe diffuse loss of toluidine blue staining with |
| | multifocal severe (depth to tide mark) chondrocyte loss and/or |
| | collagen disruption on both femurs and/or tibias, total overall |
| | loss greater than 50% of width of all surfaces combined |

### Ankle Bone Resorption

| | |
|---|---|
| 0 | Normal |
| 0.5 | Minimal resorption affects only marginal zones and affects only a |
| | few joints |
| 1 | Minimal = small areas of resorption, not readily apparent on |
| | low magnification, rare osteoclasts |
| 2 | Mild = more numerous areas of resorption, not readily apparent |
| | on low magnification, osteoclasts more numerous, <¼ of tibia or |
| | tarsals at marginal zones resorbed |
| 3 | Moderate = obvious resorption of medullary trabecular and cortical |
| | bone without full thickness defects in cortex, loss of some medullary |
| | trabeculae, lesion apparent on low magnification, osteoclasts more |
| | numerous, ¼ to ⅓ of tibia or tarsals affected at marginal zones |
| 4 | Marked = Full thickness defects in cortical bone, often with |
| | distortion of profile of remaining cortical surface, marked loss of |
| | medullary bone, numerous osteoclasts, ½ to ¾ of tibia or tarsals |
| | affected at marginal zones |
| 5 | Severe = Full thickness defects in cortical bone, often with |
| | distortion of profile of remaining cortical surface, marked loss of |
| | medullary bone, numerous osteoclasts, >¾ of tibia or tarsals affected |
| | at marginal zones, severe distortion of overall architecture |

### Knee Bone Resorption

| | |
|---|---|
| 0 | Normal |
| 0.5 | Minimal resorption affects only marginal zones |
| 1 | Minimal = small areas of resorption, not readily apparent on low |
| | magnification, approximately 1-10% of total joint width of |
| | subchondral bone affected |
| 2 | Mild = more numerous areas of resorption, definite loss of |
| | subchondral bone, approximately 11-25% of total joint width of |
| | subchondral bone affected |
| 3 | Moderate = obvious resorption of subchondral bone approximately |
| | 26-50% of total joint width of subchondral bone affected |
| 4 | Marked = obvious resorption of subchondral bone approximately |
| | 51-75% of total joint width of subchondral bone affected |
| 5 | Severe = distortion of entire joint due to destruction approximately |
| | 76-100% of total joint width of subchondral bone affected |

### Results:

Disease severity in the disease control group increased from days 1 to 5 with day 4-5 having the greatest daily increase. Then the incremental increases were smaller to the peak at day 7. From that point forward, acute swelling generally decreased and calliper measures were decreased. The treatment groups followed this general pattern as well.

Body weight loss was observed in all disease groups whereas the normal control group had a weight increase. Body weight loss was significantly (25%, p<0.05 by ANOVA) inhibited for rats treated with 5 mg/kg SEQ ID NO: 172 as compared to vehicle treated disease controls. When compared to disease controls using a Student's t-test, inhibition of body weight loss was also significant for rats treated with 1 mg/kg SEQ ID NO: 172 (21%, p<0.05) or Dex (21%, p<0.05). Results of treatment with SEQ ID NO: 172 were dose responsive for this parameter.

Daily ankle diameter measurements were significantly (p<0.05 by 2-way RM ANOVA) reduced toward normal for rats treated with 5 mg/kg SEQ ID NO: 172 (p<0.05 days 4-12) or Dex (p<0.05 d3-14) as compared to disease controls.

Ankle diameter AUC was significantly (p<0.05 by ANOVA) reduced toward normal for rats treated with 5 mg/kg SEQ ID NO: 172 (43% reduction), 1 mg/kg SEQ ID NO: 172 (27%), or Dex (97%) as compared to disease controls. Results of treatment with SEQ ID NO: 172 were dose responsive for this parameter.

Final paw weights were significantly (p<0.05 by ANOVA) reduced toward normal for rats treated with 5 mg/kg SEQ ID NO: 172 (26% reduction) or Dex (114%) as compared to disease controls.

Results of treatment with SEQ ID NO: 172 were dose responsive for this parameter. Relative liver weights were not significantly (by ANOVA) affected for rats in any treatment group as compared to disease controls.

Spleen weights relative to body weight were significantly (p<0.05 by ANOVA) reduced for rats treated with Dex as compared to disease controls. Relative spleen weights for Dex treated rats were also significantly reduced as compared to normal controls. Relative spleen weights were not significantly affected for rats treated with SEQ ID NO: 172.

Thymus weights relative to body weight were significantly (p<0.05 by ANOVA) reduced for rats treated with Dex as compared to disease controls. Relative thymus weights for Dex treated rats were also significantly reduced as compared to normal controls. Relative thymus weights were not significantly affected for rats treated with SEQ ID NO: 172.

All ankle histopathology parameters were significantly (by Mann-Whitney U test) reduced toward normal for rats treated with 5 mg/kg SEQ ID NO: 172 (25% reduction of summed scores) as compared to disease controls.

All knee histopathology parameters were significantly (by Mann-Whitney U test) reduced toward normal for rats treated with 5 mg/kg SEQ ID NO: 172 (73% reduction of summed scores) as compared to disease controls.

Results of this study indicated that daily intravenous treatment with SEQ ID NO: 172 (5 mg/kg) had significant beneficial effect on the clinical and histopathology parameters associated with established type II collagen arthritis in rats. Treatment with SEQ ID NO: 172 (1 mg/kg) resulted in significantly reduced ankle diameter AUC. The beneficial effect on ankle diameter was observed up to day 12 despite the reduction of swelling after day 7 in disease control animals. Results of treatment with SEQ ID NO: 172 were dose responsive.

Treatment with SEQ ID NO: 172 had no adverse effect on organ weights unlike dexamethasone.

### Example 12

### Effect of the all-D-Retro-Inverso JNK-Inhibitor (Poly-)Peptide of SEQ ID NO: 197 and the JNK Inhibitor (Poly-)Peptide of SEQ ID NO: 172 at Three Doses in a Scopolamine-Induced Model of Dry Eye in Mice

### Study Concept

The objective of this study was to assess the effects of two different compounds, the all-D-retro-inverso JNK-inhibitor (poly-)peptide of SEQ ID NO: 197 and the JNK inhibitor (poly-)peptide of SEQ ID NO: 172, at three dose levels in a mouse model of scopolamine-induced dry eye.

The peptides of SEQ ID NO: 197 and SEQ ID NO: 172 were tested for efficacy in this murine model of dry eye. The peptides were both tested at a low, medium and a high dose. For the peptide of SEQ ID NO: 197 the concentrations measured in the formulation samples for low, medium and high dose levels were 0.06% (w/v), 0.25% (w/v) and 0.6% (w/v), respectively, and for SEQ ID NO: 172 the concentrations measured in the formulation samples for the low, medium and high dose levels, were 0.05% (w/v), 0.2% (w/v) and 0.6% (w/v), respectively. The vehicle, which also served as the negative control, was 0.9% Sodium Chloride for Injection USP.

The study consisted of a total of 9 groups of female C57BL/6 mice, comprising 8 groups of 12 mice each and an additional group of 4 mice. Bilateral short-term dry eye was induced by a combination of scopolamine hydrobromide (Sigma-Aldrich Corp., St. Louis, Mo.) injection (subcutaneous (SC), four times daily, 0.5 mg/dose, Days 0-21) and by exposing mice to the drying environment of constant air draft. Starting on Day 1, mice of Groups 1-8 were treated three times daily (TID) for 21 days with bilateral topical ocular (oculus uterque; OU) administration (5 μL/eye/dose) of vehicle (0.9% sterile saline; negative control article); the peptide of SEQ ID NO: 197 (0.06%, 0.25% and 0.6%), the peptide of SEQ ID NO: 172 (0.05%, 0.2% and 0.6%); or cyclosporine (0.05%; positive control, an immunosuppressant drug used to reduce the activity of the immune system). Mice of Group 9 were maintained as un-induced, (no dry eye) untreated controls.

During the in-life (treatment) period, clinical observations were recorded once daily; slit-lamp examination (SLE) with corneal fluorescein staining, tear break-up time test (TBUT), and phenol red thread test (PRTT) were performed three times per week. Necropsies were performed on Day 22; eyes, eye lids, conjunctivae, and lacrimal glands were collected from both eyes of each animal. Tissues from the right eyes (oculus dexter, OD) were fixed and then evaluated microscopically. Tissues from the left eyes (oculus sinister; OS) were flash-frozen in liquid nitrogen and stored frozen at −80° C. for possible subsequent analyses.

TABLE 5
Experimental Design

| | | Induction of | Treatment |
|---|---|---|---|
| | Number of | Dry Eye | (TID, OU, |
| | animals | (QID, SC) | 5 μL/eye) |
| Group | (females) | Days 0 to 21 | Days 1 to 21 |
| 1 | 12 | Scopolamine | Vehicle |
| 2 | 12 | (200 μL of | SEQ ID NO: 197 |
| | | 2.5 mg/mL | (0.06%) |
| 3 | 12 | sol., 0.5 | SEQ ID NO: 197 |
| | | mg/dose) | (0.25%) |
| 4 | 12 | | SEQ ID NO: 197 |
| | | | (0.6%) |
| 5 | 12 | | SEQ ID NO: 172 |
| | | | (0.05%) |
| 6 | 12 | | SEQ ID NO: 172 |
| | | | (0.2%) |
| 7 | 12 | | SEQ ID NO: 172 |
| | | | (0.6%) |
| 8 | 12 | | Restasis ®* |
| | | | (0.05%) |
| 9 | 4 | No dry eye | No treatment |
| | | induction | |

*Cyclosporine

### Methods

### 1. Dose Preparation

The (poly-)peptide of SEQ ID NO: 197 was obtained from Polypeptide Laboratories (France) as a 1.5-ml clear plastic microfuge vial containing 300.65 mg of dry powder.

The (poly-)peptide of SEQ ID NO: 172 was obtained from Polypeptide Laboratories (France) as a 1.5-mL clear plastic microfuge vial containing 302.7 mg of dry powder.

Prior to the start of the study, the (poly-)peptides of SEQ ID NO: 172 and of SEQ ID NO: 197 were formulated in sterile saline (vehicle). Dosing solutions at each concentration were sterilized using 0.2-μm filters, aliquoted to multiple pre-labeled vials, and frozen at −20° C. The concentrations measured in the formulation samples for the peptide of SEQ ID NO: 197 were 0.058%, 0.25% and 0.624%, rounded to 0.06%, 0.25% and 0.6%. The concentrations measured in the formulation samples for the peptide of SEQ ID NO: 172 were 0.053%, 0.217% and 0.562%, rounded to 0.05, 0.2% and 0.6%.

On each day of dosing, one set of dosing solutions was thawed and used for that day's dose administrations. The controls (vehicle, cyclosporine) were provided ready to dose; no dose preparation was necessary.

### 2. Slit-Lamp Examinations (SLE)

Prior to entry into the study, each animal underwent a SLE and indirect ophthalmic examination using topically-applied fluorescein. Ocular findings were recorded using the Draize scale ocular scoring. SLE and Draize scoring were repeated three times a week during the in-life period.

### 3. Tear Break-Up Time (TBUT) Test and Subsequent Corneal Examination

The TBUT test was conducted three times weekly by measuring the time elapsed in seconds between a complete blink after application of fluorescein to the cornea and the appearance of the first random dry spot in the tear film. To perform the TBUT, 0.1% liquid sodium fluorescein was dropped into the conjunctival sac, the eyelids were manually closed three times and then held open revealing a continuous fluorescein-containing tear film covering the cornea, and the time (in seconds) required for the film to break (appearance of a dry spot or streak) was recorded. At least ninety seconds later, corneal epithelial damage was graded using a slit-lamp with a cobalt blue filter after another drop of 0.1% fluorescein was reapplied to the cornea; the cornea then was scored per the Draize ocular scale.

### 4. Phenol Red Thread Tear Test (PRTT)

Tear production was measured three times a week in both eyes using PRTT test strips (Zone-Quick; Menicon, Nagoya, Japan). Prior to the first treatment of the day, a thread was applied to the lateral canthus of the conjunctival fornix of each eye for 30 seconds under slit-lamp biomicroscopy. Tear migration up the tread (i.e., the length of the wetted cotton thread) was measured using a millimeter scale.

### 5. Necropsy and Pathology

At necropsy on Day 22, both eyes from each animal, including the globes, lacrimal glands, eyelids, and conjunctivae, were excised. The right eye and associated tissues were fixed by overnight submersion in modified Davidson's solution followed by transfer to 10% neutral buffered formalin (NBF). The fixed tissues of the right eye were dehydrated, embedded in paraffin, sectioned at 3 to 5-μm thicknesses, and slide-mounted tissues were stained with hematoxylin and eosin (H & E). Stained slides were evaluated via light microscopy. Detailed and complete histopathologic assessment was conducted on all parts of the eye, with at least two section levels being examined histopathologically for each right eye. Special attention was paid to the cornea, epithelia (including goblet cells) of the conjunctiva and cornea, as well as the lacrimal gland. These tissues were scored for injury based upon a 0-4 scale, with 0 being normal, 1 being minimal, 2 being mild, 3 being moderate, and 4 being severe. For each cornea, scores were based on corneal epithelium thickness, and corneal inflammation. Conjunctivae were scored for erosion and inflammation as well as presence or absence of goblet cells.

### Results

Four-times daily SC administration of scopolamine (0.5 mg/dose) induced a dry eye syndrome in female C57BL/6 mice characterized by a decrease in the volume of aqueous tear production and changes in the physiochemical properties of the tears rendering them less capable of maintaining a stable tear film able to effectively lubricate and protect the eye.

### 1. Tear Break-Up Time (TBUT) Teat and Corneal Examination

The tear break-up time tests (TBUTs) were performed prior to the induction of dry eye, and again on Days 2, 4, 7, 9, 11, 14, 16, 18 and 21 after dry eye induction. After initiation of dosing with scopolamine (dry eye induction) TBUT mean values began to decrease in all animals, but appeared to decrease more slowly in Group 6 (mid-dose of SEQ ID NO: 172). The TBUT mean nadir for Groups 5, 6, 7 (low, mid and high-dose of the peptide of SEQ ID NO: 172), and Group 8 (cyclosporine) occurred on Day 7, reaching similar values (6.6±0.4, 6.7±0.4, 6.7±0.3, and 6.4±0.4 s, respectively). Subsequently, the TBUT means of these groups increased to a peak on Day 9. Groups 6 and 7 (SEQ ID NO: 172 mid and high-dose groups) TBUT means rose to higher values (10.0±0.7 s and 9.9±0.8 s, respectively) than Group 8, the cyclosporine group (8.5±0.3 s), while the peak TBUT mean of Group 5, the low-dose of SEQ ID NO: 172 (8.0±0.4 s) was slightly below that of Group 8 (cyclosporine). TBUT means for the mid and high-dose of SEQ ID NO: 197-treated animals, Groups 3 and 4, continued to decline after onset of dosing, reaching a nadir on Day 9, while the low-dose Group 2 increased on Day 9. The low, medium and high-dose TBUT means of SEQ ID NO: 172-treated animals (Groups 2, 3 and 4, respectively) were above the vehicle group and generally below the low, mid and high-dose group means of SEQ ID NO: 172-treated animals.

When the area under the curve (AUC) for TBUT values from Day 7 to Day 21 was used to compare the various treatments with the vehicle control, treatment with mid, low and high-dose of the peptide of SEQ ID NO: 172 (0.05%, 0.2% and 0.6%, respectively), Groups 5, 6, and 7, as well as animals treated with cyclosporine (0.05%), Group 8, showed significant increases in the TBUT AUC (Kruskal-Wallis nonparametric ANOVA). The peptide of SEQ ID NO: 172 appeared to produce a dose-dependent increase in TBUT, with the mid and high-doses often producing similar effects. Furthermore, there were no significant differences in TBUT AUC between the cyclosporine-treated group, the groups treated with three dose levels of SEQ ID NO: 172 and the un-induced group (Groups 5, 6, 7, 8, and 9). This finding suggests that all three doses of the peptide of SEQ ID NO: 172 and cyclosporine were approximately equally effective in improving or reversing the ophthalmological changes that underlie the TBUT changes in this dry eye model.

Groups treated with low, mid and high dose levels of the peptide of SEQ ID NO: 197 (Groups 2-4) showed slight generally dose-dependent increases in TBUT which started to increase approximately two days later than animals treated with SEQ ID NO: 172 or cyclosporine.

TABLE 6
Mean Calculated TBUT AUC Values:

| | TBUT |
|---|---|
| Group | AUC |
| Group 1 | 71.19 |
| Group 2 | 88.54 |
| Group 3 | 91.19 |
| Group 4 | 89.98 |
| Group 5 | 102.98 |
| Group 6 | 119.08 |
| Group 7 | 119.31 |
| Group 8 | 116.1 |
| Group 9 | 124.54 |

### 2. Phenol Red Thread Tear Test (PRTT)

PRTT tests were performed prior to the induction of dry eye, and again on Days 2, 4, 7, 9, 11, 14, 16, 18 and 21. PRTT values from Day 0 to Day 4 decreased in all mice that had dry eye induced, indicating a decrease in tear production after the administration of scopolamine and exposure to a drying environment of increased air draft created by the blowers. The nadir in PRTT in most groups occurred at approximately Day 7. PRTT kept decreasing in the vehicle control group (Group 1) reaching a nadir on Day 14. After the nadir, there was an increase in all dry eye groups. These findings indicate that initiation of scopolamine treatment one day earlier than initiation of compound treatment was sufficient to initiate physiological changes in the eye associated with dry eye syndrome. Even the cyclosporine-treated group showed a decrease in PRTT similar to other groups through approximately Day 7, then increased to a peak on Days 11-14, followed by a slight decrease. In the last PRTT test (Day 21) cyclosporine (Group 8), and Groups 6 and 7 all had similar PRTT values suggesting that both the mid and high-dose of the peptide of SEQ ID NO: 172 treatments have therapeutic effects similar to cyclosporine in increasing the aqueous tear production in this murine dry eye model.

Animals treated with the low, mid or high-dose of the peptide of SEQ ID NO: 172 produced significantly more aqueous tears compared to vehicle-treated animals. Thus, similar to TBUT, the peptide of SEQ ID NO: 172 produced generally dose-related significant increases in the production of aqueous tears in this model.

Groups treated with low, mid and high dose levels of the peptide of SEQ ID NO: 197 (0.06%, 0.25% and 0.6%, Groups 2, 3 and 4, respectively) showed generally dose-dependent increases in PRTT.

TABLE 7
Mean PRTT AUC Values

| | PRTT |
|---|---|
| Group | AUC |
| Group 1 | 35.02 |
| Group 2 | 39.96 |
| Group 3 | 42.79 |
| Group 4 | 43.17 |
| Group 5 | 44.38 |
| Group 6 | 44.85 |
| Group 7 | 46.10 |
| Group 8 | 49.44 |
| Group 9 | 113.63 |

### 3. Histopathology

In this study histologic changes were generally confined to the cornea. Findings in the cornea consisted of increased keratinization of the corneal epithelial surface, increased thickness of the corneal epithelium, increased cellularity of the corneal epithelium, mildly increased incidence of mitosis of the basal epithelial layer consistent with increased epithelial cell turnover. These findings are indicative of a physiologic adaptive response to corneal drying and corneal surface irritation. Surface ulceration, corneal stromal edema and inflammatory infiltrate into the cornea were not seen in this study. The eyes in Group 9, the untreated group (normal mice, no scopolamine treatment), were within normal limits. There was some minimal nonsuppurative inflammation of the eye lids scattered throughout all groups, but the conjunctiva, retina, lacrimal glands and other parts of the eye were within normal limits. Goblet cells appeared to be within limits in all groups. Goblet cells are a primary producer of mucin which helps the tears form a stronger more adhesive film.

Mild to moderate corneal changes were noted in all groups except the untreated normal eye group (Group 9) and were slightly more severe in Group 1, the vehicle-treated group and Group 2, the low dose of the peptide of SEQ ID NO: 197, in comparison to the other treatment groups. These findings were consistent with the positive beneficial effects of increased tear production on the cornea.

When histological scores of the various treatment groups were compared to the histological scores in the cyclosporine group to determine if any other treatments produced “similar score reductions” to cyclosporine, Groups 4, 6, and 7 were found to be not significantly different than the cyclosporine group scores. Thus, these three treatments, mid and high-dose of the peptide of SEQ ID NO: 172 and the high-dose of the peptide of SEQ ID NO: 197, were the most effective, after cyclosporine, in reducing/ameliorating the corneal changes associated with this murine dry eye model.

According to another aspect of the present invention, an inventive JNK inhibitor sequence for the (in vitro) treatment of a tissue or organ transplant prior its transplantation. Usually, a transplant originating from brain-dead donors is typically not subjected to WIT has 8-12 hrs of CIT (time needed for transportation from the procurement hospital to the isolation lab. It was found that such transplants may be pre-treated by the JNK inhibitors according to the present invention in order improve their viability and functionality until transplanted to host. For that aspect of the invention, the transplant is typically a kidney, heart, lung, pancreas, liver, blood cell, bone marrow, cornea, accidental severed limb, in particular fingers, hand, foot, face, nose, bone, cardiac valve, blood vessel or intestine transplant, preferably a kidney, heart, pancreas or skin transplant.

### Example 13

### Effect of a JNK Inhibitor on Adriamycin-Induced Nephropathy in Rats

Adriamycin treatment induces glomerular disease in rat and mice mimicking human focal segmental and glomerular sclerosis (FSGS). In this model, tubular and interstitial inflammatory lesions occur during the disease course, partly due to heavy proteinuria. In the absence of therapy, kidney disease progresses to terminal renal failure within eight weeks. Podocyte injury is one of the initial steps in the sequences leading to glomerulosclerosis. The aim of the study was to investigate whether a JNK inhibitor could prevent the development of renal lesions and the renal failure.

### Methods

30 male Sprague-Dawley rats (Charles River) were used in this study (divided into 3 groups of ten rats). Nephropathy was induced by a single intravenous injection of Adriamycin 10 mg/kg on Day 0. The JNK inhibitor of SEQ ID NO: 172 (2 mg/kg; in NaCl 0.9%) was administered intravenously in the tail vein on Day 0. The administration volume was 0.2 ml.

The table below summarizes the random allocation:

| | | | Dose volume/ | Dose | Number |
|---|---|---|---|---|---|
| Group | ADR | Treatment | Route of | concen- | of |
| No | (Day 0) | (Day 0) | administration | tration | animals |
| 1 | 10 | NaCl | 0.2 | 0 | 10 |
| | mg/kg | 0.9% | ml, IV | | |
| 2 | 10 | JNK inhibitor of | 0.2 | 1 | 10 |
| | mg/kg | SEQ ID NO: 172 | ml, IV | mg/ml | |
| | | 2 mg/kg | | | |
| 3 | NaCl | NaCl | 0.2 | 0 | 10 |
| | 0.9% | 0.9% | ml, IV | | |

Each day, the general behavior and the appearance of all animals were observed. The health of the animals was monitored (moribund animals, abnormal important loss of weight, major intolerance of the substance, etc. . . . ). No rats were removed.

Blood was collected from the tail vein at Days 7, 14, 28, 42 and 56 from 4 rats per group. Serum creatinine concentrations, blood urea and protidemia were measured using appropriate kits from Advia Chemistry 1650 (Bayer Healthcare AG, Leverkusen, Germany).

Two rats per group were sacrificed on Days 7, 14, 28, 42 and 56 after anesthesia. After animal sacrifice, both kidneys were collected. For histopathological examination fixed tissue specimens were dehydrated in graded alcohol solutions, cleared in toluene, and embedded in paraffin. Sections (4 μm) were stained with periodic acid-Schiff (PAS), and Masson's trichrome staining was performed to detect collagen deposition. Glomerular and tubulointerstitial sclerosis were quantified under microscope.

Results were expressed in the form of individual and summarized data tables using Microsoft Excel® Software. Numerical results were expressed as mean±standard error of the mean (SEM). Due to the small number of animal tested, no statistical analyses was performed.

### Results:

Effect of the JNK inhibitor of SEQ ID NO: 172 on renal function during the progression of the disease: Urea and creatinine serum levels were measured to study the renal function during the kidney disease course. Because creatinine interferes with the calorimetric dosage, only urea that is a fine indicator of renal function was analyzed. Whereas urea serum levels were remarkably stable in untreated rats (below 5 mmol/l), ADR induced progressive increase of urea levels, which sharply raised from Day 28 up to 25 mmol/l at Day 41, then 48 mmol/l at Day 56 reflecting terminal renal failure (FIG. 38 B). On the other hand, JNK inhibitor of SEQ ID NO: 172-treated rats exhibited an urea serum level below 10 mmol/l throughout the course of the disease (FIG. 38 B). These results suggest that JNK inhibitor of SEQ ID NO: 172 prevents the progression to renal disease and renal failure.

### Histopathological Findings (PAS and Masson Trichrome Staining):

ADR-induced structural changes were evaluated under light microscope. Saline-treated control rats showed morphologically normal glomeruli and tubules. On Day 8, light microscopic examination showed some areas with focal segmental glomerulosclerosis and proteinaceous casts in the ADR nephrosis group. In contrast, although some tubules were filled with proteins in JNK inhibitor of SEQ ID NO: 172-treated rats, glomeruli exhibited a normal architecture with absence or discrete mesangial hypercellularity, while the tubular structures and interstitium did not display pathological changes (FIG. 39). By Day 14, ADR treated rats exhibited progressive glomerulosclerosis, hyaline deposits, tubular dilation and cast formation. The degree of glomerulosclerosis was dramatically worsened in this group and became diffuse with obvious adhesion between the glomerular tufts and the Bowman's space in most glomeruli by Day 29 and 41, associated with severe tubular atrophy and interstitial fibrosis. At Day 56, diffuse glomerular sclerosis was observed in all glomeruli (FIG. 40). However, JNK inhibitor of SEQ ID NO: 172-treated rats had a relatively normal appearance at Day 8, and develop few focal and segmental glomerulosclerosis and tubulointerstitial fibrosis at Day 56 compared with ADR-treated rats. Altogether, these results strongly suggest that the JNK inhibitor of SEQ ID NO: 172 prevents the development of glomerular and tubulointerstitial fibrosis and may explain the preservation of renal function in this group.

The study results provide evidence that the JNK inhibitor of SEQ ID NO: 172 prevents the progression of glomerular and tubulointerstitial injuries induced by ADR. Moreover, this molecule preserves renal function.

### Example 14

### Evaluation of a JNK Inhibitor on Iquimod-Induced Psoriasis in Mice

Imiquimod (IMQ), a ligand for TLR7 and TLR8, is a potent immune response modifier. It has been demonstrated for potent antiviral and antitumor effects in many animal models. Van der Fits et al. (The Journal of Immunology 2009, 182, P. 5836-5845) have demonstrated that the topical application of IMQ in BALB/c mice induced psoriasis and closely resemble human psoriasis lesion.

### Methods

Female BALB/cAnNCrl mice (Charles River, age 8 to 10 weeks at study start) have been assigned to the following groups (treatment schedule):

| | | Dose | Prep | Dosing | | | |
|---|---|---|---|---|---|---|---|
| | Dose | Volume | Conc. | Duration | Prep | | No. of |
| Group | (mg/kg) | (ml/kg) | (mg/ml) | (# Days) | Frequency | Route | animals |
| Vehicle | N/A | 5 | N/A | Days 1, 4, 7 | Days 1, 4, 7 | IV | 8 |
| SEQ ID NO: 172 | 0.02 | 5 | 0.004 | Days 1, 4, 7 | Days 1, 4, 7 | IV | 8 |
| SEQ ID NO: 172 | 0.2 | 5 | 0.04 | Days 1, 4, 7 | Days 1, 4, 7 | IV | 8 |
| SEQ ID NO: 172 | 2 | 5 | 0.4 | Days 1, 4, 7 | Days 1, 4, 7 | IV | 8 |
| Prednisolone | 10 | 10 | 1 | 7 | Daily | PO | 8 |
| Dexamethasone | 0.5 | 5 | 0.1 | Days 1, 4, 7 | Days 1, 4, 7 | IV | 6 |

Additionally, a group of five animals has not been treated (“Naïve” group).

To demonstrate whether topical application of IMQ induced skin inflammation is accompanied by structural features characteristic for psoriasis, IMQ cream (approx. 62.5 mg Imiquimod Cream 5%) has been applied on the back of shaved skin and to the right ear of the BALB/c mice for 6 consecutive days (days 2 through 7).

In this experiment, two positive controls have been utilized. Firstly, Prednisolone at 10 mg/kg (vehicle: 1% Hydroxyethylcellulose, 0.25% Polysorbate 80, and 0.05% Antifoam in purified water) has been dosed daily and orally (group “Prednisolone”). Secondly, Dexamethasone has been administered at 0.5 mg/kg (vehicle: sterile 0.9% NaCl) on days 1, 4 and 7 via intravenous route.

The JNK inhibitor of SEQ ID NO: 172 (“SEQ ID NO: 172”) has been dissolved in 0.9% NaCl. To receive three different doses (cf. above, groups table) it has been serially diluted (1:10 fold). The JNK inhibitor of SEQ ID NO: 172 was readily soluble and did not fall out of solution. The three different doses of the JNK inhibitor of SEQ ID NO: 172 (0.02, 0.2 and 2 mg/kg) have been administered to the respective groups intravenously on days 1, 4 and 7.

On day 8, animals have been sacrificed and the tissue (ear) has been fixed in 10% neutral buffered formalin. For histopathology hematoxylin-and-eosin-stained sections (cross cut) have been prepared and microscopic evaluation on the collected tissues from all animals has been performed. Methods and end-points for histopathology were similarly described in the van der Fits (2009) paper in that inflammation, epidermal hyperplasia, epidermal hyperkeratosis (rather than parakeratosis) were observed and recorded for severity grade, whereby the respective methodology from Van der Fits et al. (The Journal of Immunology 2009, 182, P. 5836-5845) is hereby incorporated by reference. Histopathology grading scores were excluded for either skin or ear in animals with secondary inflammatory processes (full thickness epidermal ulcers). Scores were averaged by group and standard deviation and statistical significance were calculated. The graph in FIG. 41 shows group averages (+/−) standard deviation (SD) are depicted below. Formalin-fixed, paraffin embedded skin from the dorsal surface of the mouse (BALB/c) was stained with hematoxylin and eosin (H&E) stain and assessed microscopically. An important difference from the above reference and to describe in more detail the observations of the present study: Hyperkeratosis can be defined specifically as orthokeratotic (no retained nuclei) or parakeratotic (retained nuclei). Either can occur normally in various anatomical locations and depending on species; however, both conditions are well defined in particular disease states. The van der Fits paper describes their Imiquimod (IMQ)-induced psoriasis model as causing parakeratotic hyperkeratosis similar to what is seen in the human condition, and that was a defined end-point for this study. However, the Danilenko et al. (Veterinary Pathology 2008 45:563) has shown that many rodent psoriasis models have orthokeratotic hyperkeratosis. In reality, the same lesion can sometimes exhibit both types of hyperkeratosis, and the rodents in this study had primarily orthokeratotic hyperkeratosis with rare, multifocal parakeratosis. The more general term ‘hyperkeratosis’ was used for grading end-points and describe in the text what type was seen (primarily orthokeratotic). Another difference from the van der Fits paper, is that they describe human patients as having decreased granulation in their stratum granulosum layer of the epidermis (and in their study, the rodent skin was reportedly similar); however, in this study, and the Danilenko review, many rodent models of psoriasis exhibit increased (hypergranulosis) granulation in this layer or the layer itself is hyperplastic.

Microscopic Histopathology end-points were graded as such:

1=MI=minimal
2=SL=slight
3=M0=moderate
4=MA=marked
5=SE=severe

### Results

The JNK inhibitor of SEQ ID NO: 172 mid-dose group (statistically significant) and the JNK inhibitor of SEQ ID NO: 172 high-dose group had decreased inflammation of the ear compared to the vehicle-IMQ dose group (FIG. 41). Also the positive control groups, i.e. the Prednisolone group and the Dexamethasone group, showed decreased inflammation of the ear compared to the vehicle-IMQ dose group (both statistically significant, FIG. 41). In general, inflammation that was present in the dermis consisted of lymphocytes and macrophages admixed with fewer neutrophils. Inflammation in the epidermis, which was much less common, was primarily neutrophilic and was presentin intracorneal layers (of orthokeratotic layers) and in the intraepidermis as Munro's microabscesses. Inflammation was not present in the naïve group.

Minimal decreases in epidermal hyperplasia of the ear were also observed for the JNK inhibitor of SEQ ID NO: 172 mid-dose group that was slightly below that observed for the Prednisolone and Dexamethasone groups. Although the JNK inhibitor of SEQ ID NO: 172 mid-dose and prednisolone groups were below that of the vehicle-IMQ dose group, they were not statistically significant. No overt differences were exhibited as a dose-response treated with JNK inhibitor of SEQ ID NO: 172 for ear with regards to epidermal hyperkeratosis, however the JNK inhibitor of SEQ ID NO: 172 low-dose group, Prednisolone, and Dexamethasone groups had minimally decreased average grades compared to the vehicle-IMQ dose group. The naïve group was microscopically normal.

### Example 15

### Effects of a JNK Inhibitor on Renal Ischemia/Reperfusion Lesions

The aim of this study is to investigate the influence of the JNK inhibitor of SEQ ID NO: 172 on experimental renal ischemia in rats.

To this end, 26 male Wistar rats (age 5 to 6 weeks, Charles River) are assigned to the following groups:

| | | | | | Renal | |
|---|---|---|---|---|---|---|
| | Pretreament | Treatment | Dose volume/ | | Ischemia | Number |
| Group | (1 hour before | (1 hour after | Route of | | time | of |
| No | clamping) | clamping) | administration | Concentration | (min) | animals |
| 1 | Heparine | NaCl 0.9% | 2 ml/kg, IV | 0 | | 6 |
| | (5000 UI/kg) | | | | | |
| 2 | Heparine | JNK inhibitor | 2 ml/kg, IV | 1 mg/ml | 40 | 10 |
| | (5000 UI/kg) | SEQ ID NO: 172 | | | | |
| | | 2000 μg/kg | | | | |
| 3 | Heparine | NaCl 0.9% | 2 ml/kg, IV | 0 | 40 | 10 |
| | (5000 UI/kg) | | | | | |

Renal ischemia will be induced by clamping both renal pedicles with atraumatic clamp (induction of necropathy). One unique dose of the JNK inhibitor of SEQ ID NO: 172 (2000 μg/kg) will be administered intravenously (IV) into the tail vein on Day 0, one hour after clamping period (after reperfusion) both renal pedicles with atraumatic clamp. The administration volume will be 2 ml/kg. Heparin (5000 UI/kg) will be administered intraperitoneally 1 hour before clamping.

Each day, the general behavior and the appearance of all animals is observed. If animal health is not compatible with the continuation of the study (moribund animals, abnormal important loss of weight, major intolerance of the substance, etc. . . . ), animals will be ethically sacrificed under the responsibility of the Study Director. Individual rats are housed in metabolic cages (Techniplast, France). Urine is collected every 24 hours up to 72 hours. Blood samples are obtained from tail vein before, then at 24 and 72 hours after reperfusion. At the end of both periods (24 and 72 hours), 5 rats per group (3 for group 1) are sacrificed. After animal sacrifice, both kidneys are collected. Five rats per group (3 for group 1) are used at each time point (24 and 72 hours after reperfusion). For the evaluation of the renal function, serum creatinine (pmol/ml) or urea concentrations (mmol/mL) are measured with the appropriate kits (Bayer Healthcare AG, Leverkusen, Germany). For the evaluation of proteinuria and albuminuria, proteinuria and albuminuria are performed using appropriate kits from Advia Chemistry 1650 (Bayer Healthcare AG, Leverkusen, Germany).

Evaluation of histological lesions is performed 24 and 72 hours after reperfusion. For light microscopy, kidneys are incubated for 16 hours in Dubosq-Brazil, dehydrated, embedded in paraffin, cut into sections and stained with hematoxylin and eosin (H&E) or periodic acid-Schiff (PAS) reagent. Three sections will be analyzed for each staining.

For immunohistochemistry analysis, kidney samples are fixed for 16 hours in Dubosq Brazil, and subsequently dehydrated and embedded in paraffin. Antigen retrieval is performed by immersing the slides in boiling 0.01 M citrate buffer in a 500 W microwave oven for 15 min. The endogenous peroxidase activity is blocked with 0.3% H₂O₂ in methanol for 30 min. Slides are incubated with the blocking reagents consisting of the Avidin-biotin solution for 30 min and the normal blocking serum for 20 min. For immunodetection, the slides are incubated overnight with an antibody, then with a biotinylated secondary antibody. An avidin-biotinylated horseradish peroxidase complex (Vectastain ABC Reagent, Vector Laboratories; Burlingame, Calif.) and 3,3′-diaminobenzidine (Sigma Biochemicals; St Louis, Mo.) as a chromogen is applied for visualization of the immunoreaction. Slides are counterstained with hematoxylin. Omission of the primary antibody is considered as a negative control.

Immunofluorescence labeling is carried out on 4 mm thick cryostat sections of kidney tissue fixed in acetone for 10 min, air-dried for 30 min at room temperature, then incubated in PBS for 3 min and blocked in 1% BSA in PBS. The sections are incubated with the indicated antibodies for 1 hour at room temperature, washed in PBS and incubated with Red Texas-conjugated secondary antibodies. Sections are examined by fluorescence microscopy (Zeiss) for immunofluorescence analysis.

The expression of several markers specific of podocyte damage, inflammation and renal fibrosis (RelA, TGF, TNF Masson trichrome) is evaluated by immunohistochemistry and immunofluorescence. Quantitative transcription profile of TNF IL6, CXCL1 (KC), CXCL2 (MIP-2) and MCP1 in kidneys are determined.

### Example 16

### Inhibitory Effects of a JNK Inhibitor on the Inflammatory Response in a Rat Periodontitis Model

The aim of this study is to investigate the influence of the JNK inhibitor of SEQ ID NO: 172 on inflammation induced in a periodontitis model in the rat.

30 rats are used in this study (divided into 4 groups of ten rats). Experimental periodontitis is induced by a ligature placed around the 1^{st} molar (one molar per animal) on Day 0. One dose of 2 or 4 mg/kg is administered intragingivally (IGV).

The table below summarizes the random allocation:

| Group | Ligature | | Route of | Number of |
|---|---|---|---|---|
| No | (Day 0) | Treatment | administration | animals |
| 1 | — | — | IGV | 10 |
| 2 | Yes | NaCl 0.9% | IGV | 10 |
| 3 | Yes | SEQ ID NO: 172 | IGV | 10 |
| | | 2 or 4 mg/kg | | |

Each day, the general behavior and the appearance of all animals is observed. It animal health is not compatible with the continuation of the study (moribund animals, abnormal important loss of weight, major intolerance of the substance, etc. . . . ), animals are ethically sacrificed under the responsibility of the Study Director. Periodontitis inflammation aspect are analyzed by macroscopic observation of gingival tissue. Plaque index and gingival inflammation index are measured as periodontal clinical indices.

Approximately on day 17 the animals are sacrificed and samples are collected. For the evaluation of inflammatory cells, quantification of inflammatory cells is performed by histomorphometric measurements. For the evaluation of inflammatory protein levels, the level of inflammatory proteins (p-JNK, TNF, IL-1, IL-10, MMP-8, MMP-9) are measured from gingival tissue homogenates. For the evaluation of tissue destruction, bone tissue destruction is evaluated on 3 animals per group by radiological analysis (micro-CT). Periodontal complex destruction is evaluated by histological analysis. For the evaluation of bone microarchitecture, bone trabecular measurements (thickness, separation) are evaluated by radiological analysis (micro-CT). For the identification of oral bacteria, bacterial population in dental pockets are identified by DNA probes (real time PCR) on 9 periodontopathogens. For the collagen framework, measurements of total collagen amount are performed using Polarized-light microscopy. The collagen I/collagen III ratio is evaluated by histomorphometrical analysis.

1. JNK inhibitor, selected from the group consisting of:
a) a JNK inhibitor, which comprises an inhibitory (poly-)peptide sequence according to the following general formula:
(SEQ ID NO: 1)
X1-X2-X3-R-X4-X5-X6-L-X7-L-X8,
wherein X1 is an amino acid selected from amino acids R, P, Q and r,
wherein X2 is an amino acid selected from amino acids R, P, G and r,
wherein X3 is an amino acid selected from amino acids K, R, k and r,
wherein X4 is an amino acid selected from amino acids P and K,
wherein X5 is an amino acid selected from amino acids T, a, s, q, k or is absent,
wherein X6 is an amino acid selected from amino acids T, D and A,
wherein X7 is an amino acid selected from amino acids N, n, r and K; and
wherein X8 is an amino acid selected from F, f and w, and
wherein an amino acid residue given in capital letters indicates an L-amino acid, while an amino acid residue given in small letters indicates a D amino acid residue,
with the proviso that at least one of the amino acids selected from the group consisting of X1, X2, X3, X5, X7 and X8 is/are a D-amino acid(s), and
b) a JNK inhibitor which comprises an inhibitory (poly-)peptide sequence sharing at least at least 80% sequence identity with SEQ ID NO: 1 as defined in a), with the proviso that with respect to SEQ ID NO: 1 such inhibitory (poly-)peptide sequence sharing sequence identity with SEQ ID NO: 1 maintains the L-arginine (R) residue of SEQ ID NO: 1 at position 4 and the two L-leucine (L) residues of SEQ ID NO: 1 at positions 8 and 10 and that at least one of the remaining amino acids in said sequence sharing at least at least 80% sequence identity with SEQ ID NO: 1 is a D-amino acid,
for use in the treatment of the human or animal body of diseases.

2. JNK inhibitor for use according to claim 1, wherein at least one of the amino acids selected from the group consisting of X3, X5, X7 and X8 is/are a D-amino acid(s).

3. JNK inhibitor for use according to claim 1 or 2, wherein the JNK inhibitor comprises an inhibitory (poly-)peptide sequence sharing at least 80% sequence identity with a sequence selected from any one of SEQ ID NOs: 2-27.

4. JNK inhibitor for use according to anyone of the preceding claims, wherein the inhibitory (poly-)peptide sequence is selected from anyone of SEQ ID NOs: 2-27.

5. JNK inhibitor for use according to anyone of the preceding claims, wherein the JNK inhibitor comprises SEQ ID NO: 8 or an inhibitory (poly-)peptide sequence sharing at least 80% sequence identity with SEQ ID NO: 8.

6. JNK inhibitor for use according to anyone of the preceding claims, wherein the JNK inhibitor comprises a transporter sequence.

7. JNK inhibitor for use according to claim 6, wherein the inhibitory (poly-)peptide sequence and the transporter sequence overlap.

8. JNK inhibitor for use according to claim 6 or 7, wherein the transporter sequence comprises a sequence of alternating D- and L-amino acids according to anyone of SEQ ID NOs: 28-30.

9. JNK inhibitor for use according to anyone of claims 6-8, wherein said transporter sequence is selected from any one of SEQ ID NOs: 31-170.

10. JNK inhibitor for use according to anyone of claims 6-9, wherein said transporter sequence is selected from any one of SEQ ID NOs: 31-34, 46, 47 and 52-151.

11. JNK inhibitor for use according to anyone of claims 6-10, wherein said transporter sequence is positioned directly N-terminal or directly C-terminal of the inhibitory (poly-)peptide sequence.

12. JNK inhibitor for use according to anyone of claims 6-11, wherein the JNK inhibitor comprises
a) a sequence according to any one of SEQ ID NOs: 171-190, or
b) a sequence sharing at least 50% sequence identity with at least one of SEQ ID NOs: 171-190, with the proviso that said sequence sharing sequence identity anyone of SEQ ID NOs: 171-190:
i) maintains the L-arginine (R) residue on position 4 in its sequence stretch corresponding to SEQ ID NO: 1,
ii) maintains the two L-leucine (L) in its sequence stretch corresponding to SEQ ID NO: 1, and
iii) exhibits at least one D-amino acid at positions X1, X2, X3, X5, X7 or X8 in its sequence stretch corresponding to SEQ ID NO: 1.

13. JNK inhibitor for use according to anyone of claims 6-12, wherein the JNK inhibitor comprises
a) the sequence of SEQ ID NO: 172 or
b) a sequence sharing 50% sequence identity with SEQ ID NO: 172, with the proviso that said sequence sharing 50% sequence identity with SEQ ID NO: 172
i) maintains the L-arginine (R) residue on position 4 in its sequence stretch corresponding to SEQ ID NO: 1,
ii) maintains the two L-leucine (L) in its sequence stretch corresponding to SEQ ID NO: 1, and
iii) exhibits at least one D-amino acid at positions X1, X2, X3, X5, X7 or X8 in its sequence stretch corresponding to SEQ ID NO: 1.

14. JNK inhibitor comprising:
a) an inhibitory (poly-)peptide comprising a sequence selected from the group of sequences consisting of RPTTLNLF (SEQ ID NO: 191), KRPTTLNLF (SEQ ID NO: 192), RRPTTLNLF and/or RPKRPTTLNLF (SEQ ID NO: 193), and
b) a transporter sequence selected from SEQ ID NOs: 31-34 and 46-151,
for use in a method for treatment of the human or animal body by therapy.

15. JNK inhibitor comprising the sequence of SEQ ID NO: 194 or 195 for use in a method for treatment of the human or animal body by therapy.

16. JNK inhibitor for use according to any one of claims 1 to 15, wherein said method is for treatment of the human body by therapy.

17. JNK inhibitor for use according to any one of claims 1 to 16, wherein said JNK inhibitor is administered intravenously, intramuscularly, subcutaneously, intradermally, transdermally, enterally, orally, rectally, topically, nasally, locally, intranasally, epidermally, by patch delivery, by instillation, intravitreally, subconjunctivally and/or intratympanically.

18. JNK inhibitor for use according to any one of claims 1 to 17, wherein said use is for treatment of kidney diseases and/or disorders in particular selected from glomerulonephritis in general, in particular membrano-proliferative glomerulonephritis, mesangio-proliferative glomerulonephritis, rapidly progressive glomerulonephritis, nephrophathies in general, in particular membranous nephropathy or diabetic nephropathy, nephritis in general, in particular lupus nephritis, pyelonephritis, interstitial nephritis, tubulointerstitial nephritis, chronic nephritis or acute nephritis, and minimal change disease and focal segmental glomerulosclerosis.

19. JNK inhibitor for use according to any one of claims 1 to 17, wherein said use is for treatment of diseases and/or disorders selected from skin diseases, in particular inflammatory skin diseases, more specifically skin diseases selected from the group consisting of eczema, psoriasis, dermatitis, acne, mouth ulcers, erythema, Lichen plan, sarcoidosis, vascularitis and adult linear IgA disease, in particular atopic dermatitis or contact dermatitis.

20. JNK inhibitor for use according to any one of claims 1 to 17, wherein said use is for treatment of diseases and/or disorders selected Addison's disease, Agammaglobulinemia, Alopecia areata, Amytrophic lateral sclerosis, Antiphospholipid syndrome, Atopic allergy, Autoimmune aplastic anemia, Autoimmune cardiomyopathy, Autoimmune enteropathy, Autoimmune hemolytic anemia, Autoimmune inner ear, disease, Autoimmune lymphoproliferative syndrome, Autoimmune polyendocrine syndrome, Autoimmune progesterone dermatitis, Idiopathic thrombocytopenic purpura, Autoimmune urticaria, Balo concentric sclerosis, Bullous pemphigoid, Castleman's disease, Cicatricial pemphigoid, Cold agglutinin disease, Complement component 2 deficiency associated disease, Cushing's syndrome, Dagos disease, Adiposis dolorosa, Eosinophilic pneumonia, Epidermolysis bullosa acquisita, Hemolytic disease of the newborn, Cryoglobulinemia, Evans syndrome, Fibrodysplasia ossificans progressive, Gastrointestinal pemphigoid, Goodpasture's syndrome, Hashimoto's encephalopathy, Gestational pemphigoid, Hughes-stovin syndrome, Hypogammaglobulinemia, Lambert-eaton myasthenic syndrome, Lichen sclerosus, Morphea, Pityriasis lichenoides et varioliformis acuta, Myasthenia gravis, Narcolepsy, Neuromyotonia, Opsoclonus myoclonus syndrome, Paraneoplastic cerebellar degeneration, Paroxysmal nocturnal hemoglobinuria, Parry-romberg syndrome, Pernicious anemia, POEMS syndrome, Pyoderma gangrenosum, Pure red cell aplasia, Raynaud's phenomenon, Restless legs syndrome, Retroperitoneal fibrosis, Autoimmune polyendocrine syndrome type 2, Stiff person syndrome, Susac's syndrome, Febrile neutrophilic dermatosis, Sydenham's chorea, Thrombocytopenia, and vitiligo.

21. JNK inhibitor for use according to any one of claims 1 to 17, wherein said use is for treatment of inflammatory diseases and/or disorders selected from acute disseminated encephalomyelitis, antisynthetase syndrome, autoimmune hepatitis, autoimmune peripheral neuropathy, pancreatitis, in particular autoimmune pancreatitis, Bickerstaff's encephalitis, Blau syndrome, Coeliac disease, Chagas disease, chronic inflammatory demyelinating polyneuropathy, osteomyelitis, in particular chronic recurrent multifocal osteomyelitis, Churg-Strauss syndrome, Cogan syndrome, giant-cell arteritis, CREST syndrome, vasculitis, in particular cutaneous small-vessel vasculitis or urticarial vasculitis, dermatitis, in particular dermatitis herpetiformis, dermatomyositis, systemic scleroderma, Dressler's syndrome, drug-induced lupus erythematosus, discoid lupus erythematosus, enthesitis, eosinophilic fasciitis, gastroenteritis, in particular, eosinophilic gastroenteritis, erythema nodosum, idiopathic pulmonary fibrosis, gastritis, Grave's disease, Guillain-barré syndrome, Hashimoto's thyroiditis, Henoch-Schonlein purpura, Hidradenitis suppurativa, idiopathic inflammatory demyelinating diseases, myositis, in particular inclusion body myositis, cystitis, Kawasaki disease, Lichen planus, lupoid hepatitis, Majeed syndrome, Ménière's disease, Microscopic polyangiitis, mixed connective tissue disease, myelitis, in particular neuromyelitis, e.g. neuromyelitis optica, thyroiditis, in particular Ord's thyroiditis, rheumatism, in particular palindromic rheumatism, Parsonage-Turner syndrome, perivenous encephalomyelitis, polyarteritis nodosa, polymyalgia rheumatica, polymyositis, cirrhosis, in particular primary biliary cirrhosis, cholangitis, in particular primary sclerosing cholangitis, progressive inflammatory neuropathy, Rasmussen's encephalitis, chondritis, in particular polychondritis, e.g. relapsing polychondritis, reactive arthritis (Reiter disease), rheumatic fever, sarcoidosis, Schnitzler syndrome, serum sickness, spondylitis, in particular ankylosing spondylitis, spondyloarthropathy, Takayasu's arteritis, Tolosa-Hunt syndrome, transverse myelitis, and granulomatosis, in particular Wegener's granulomatosis.

22. JNK inhibitor for use according to any one of claims 1 to 17, wherein said use is for treatment of diseases and/or disorders selected from tauopathies and amyloidoses and prion diseases.

23. JNK inhibitor for use according to any one of claims 1 to 17, wherein said use is for treatment of polypes.

24. JNK inhibitor for use according to any one of claims 1 to 17, wherein said use is for treatment of diseases and/or disorders selected from gingivitis, osteonecrosis (e.g. of the jaw bone), peri-implantitis, pulpitis, and periodontitis.

25. JNK inhibitor for use according to any one of claims 1 to 17, wherein said use is for treatment of fibrotic diseases and/or disorders particularly selected from lung, heart, liver, bone marrow, mediastinum, retroperitoneum, skin, intestine, joint, and shoulder fibrosis.

26. JNK inhibitor for use according to any one of claims 1 to 17, wherein said use is for treatment of diseases and/or disorders selected from various forms of dementia, e.g. frontotemporal dementia and dementia with lewy bodies, schizophrenia, spinocerebellar ataxia, spinocerebellar atrophy, multiple system atrophy, motor neuron disease, corticobasal degeneration, progressive supranuclear palsy or hereditary spastic paraparesis.

27. JNK inhibitor for use according to any one of claims 1 to 17, wherein said use is for treatment of eye-related diseases and/or disorders selected from inflammation after corneal surgery, non-infective keratitis, chorioretinal inflammation, and sympathetic ophthalmia.

28. JNK inhibitor for use according to any one of claims 1 to 17, wherein said use is for treatment of diseases and/or disorders resulting from tissue or organ transplantation, in particular selected from heart, kidney, and skin (tissue), lung, pancreas, liver, blood cells, bone marrow, cornea, accidental severed limbs (fingers, hand, foot, face, nose etc.), bones of whatever type, cardiac valve, blood vessels, and segments of the intestine transplantation.

29. JNK inhibitor for use according to any one of claims 1 to 17, wherein said use is for treatment of psoriasis.

30. JNK inhibitor for use according to any one of claims 1 to 17, wherein said use is for treatment of dry eye disease.

31. JNK inhibitor for use according to any one of claims 1 to 17, wherein said use is for treatment of persistent or acute inflammatory diseases damaging the retina of the eye (retinopathy), in particular diabetic retinopathy, arterial hypertension induced hypertensive retinopathy, radiation induced retinopathy, sun-induced solar retinopathy, trauma-induced retinopathy, e.g. Purtscher's retinopathy, retinopathy of prematurity (ROP) and hyperviscosity-related retinopathy.

32. JNK inhibitor for use according to any one of claims 1 to 17, wherein said use is for treatment of age-related macular degeneration (AMD), in particular the wet or the dry form of age-related macular degeneration.

33. JNK inhibitor for use according to any one of claims 1 to 17, wherein said use is for treatment of organ transplantation, in particular upon heart, kidney, and skin (tissue) transplantation, graft rejection upon heart, kidney or skin (tissue) transplantation.

34. JNK inhibitor for use according to any one of claims 1 to 17, wherein said use is for treatment of arthrosis/arthritis, in particular reactive arthritis, rheumatoid arthrosis, juvenile idiopathic arthritis, and psoriatic arthritis.

35. JNK inhibitor for use according to any one of claims 1 to 17, wherein said use is for treatment of glomerulonephritis.

36. JNK inhibitor for use according to any one of the preceding claims, wherein the JNK inhibitor consists of the sequence of SEQ ID NO: 172.

37. Pharmaceutical composition comprising a JNK inhibitor as defined in any of claims 1 to 15 and a pharmaceutically acceptable carrier.

38. Pharmaceutical composition according to claim 37 for use for the treatment of any of the diseases/disorders of claims 18 to 35.


SEQUENCE LISTING (200 sequences)

SEQ ID NO: 1 - protein, 11 aa, Artificial
  Consensus new JNK inhibitors
XXXRXXXLXLX

SEQ ID NO: 2 - protein, 11 aa, Artificial
  rPKRPTTLNLF JNK inhibitor
RPKRPTTLNLF

SEQ ID NO: 3 - protein, 11 aa, Artificial
  RPkRPTTLNLF JNK inhibitor
RPKRPTTLNLF

SEQ ID NO: 4 - protein, 11 aa, Artificial
  RPKRPaTLNLF JNK inhibitor
RPKRPATLNLF

SEQ ID NO: 5 - protein, 11 aa, Artificial
  RPKRPTTLnLF JNK inhibitor
RPKRPTTLNLF

SEQ ID NO: 6 - protein, 11 aa, Artificial
  RPKRPTTLrLF JNK inhibitor
RPKRPTTLRLF

SEQ ID NO: 7 - protein, 11 aa, Artificial
  RPKRPTTLNLf JNK inhibitor
RPKRPTTLNLF

SEQ ID NO: 8 - protein, 11 aa, Artificial
  RPkRPaTLNLf JNK inhibitor
RPKRPATLNLF

SEQ ID NO: 9 - protein, 11 aa, Artificial
  RPkRPTTLNLf JNK inhibitor
RPKRPTTLNLF

SEQ ID NO: 10 - protein, 11 aa, Artificial
  RPkRPTTLrLf JNK inhibitor
RPKRPTTLRLF

SEQ ID NO: 11 - protein, 11 aa, Artificial
  RRrRPTTLNLf JNK inhibitor
RRRRPTTLNLF

SEQ ID NO: 12 - protein, 11 aa, Artificial
  QRrRPTTLNLf JNK inhibitor
QRRRPTTLNLF

SEQ ID NO: 13 - protein, 11 aa, Artificial
  RPkRPTTLNLw JNK inhibitor
RPKRPTTLNLW

SEQ ID NO: 14 - protein, 11 aa, Artificial
  RPkRPTDLNLf JNK inhibitor
RPKRPTDLNLF

SEQ ID NO: 15 - protein, 11 aa, Artificial
  RRrRPTTLrLw JNK inhibitor
RRRRPTTLRLW

SEQ ID NO: 16 - protein, 11 aa, Artificial
  QRrRPTTLrLw JNK inhibitor
QRRRPTTLRLW

SEQ ID NO: 17 - protein, 11 aa, Artificial
  RRrRPTDLrLw JNK inhibitor
RRRRPTDLRLW

SEQ ID NO: 18 - protein, 11 aa, Artificial
  QRrRPTDLrLw JNK inhibitor
QRRRPTDLRLW

SEQ ID NO: 19 - protein, 11 aa, Artificial
  RRrRPaTLNLf JNK inhibitor
RRRRPATLNLF

SEQ ID NO: 20 - protein, 11 aa, Artificial
  QRrRPaTLNLf JNK inhibitor
QRRRPATLNLF

SEQ ID NO: 21 - protein, 11 aa, Artificial
  RrKRPaTLNLf JNK inhibitor
RRKRPATLNLF

SEQ ID NO: 22 - protein, 11 aa, Artificial
  RPkRPsTLNLf JNK inhibitor
RPKRPSTLNLF

SEQ ID NO: 23 - protein, 11 aa, Artificial
  RPkRPqTLNLf JNK inhibitor
RPKRPQTLNLF

SEQ ID NO: 24 - protein, 11 aa, Artificial
  RPkRPkTLNLf JNK inhibitor
RPKRPKTLNLF

SEQ ID NO: 25 - protein, 10 aa, Artificial
  rGKRKALKLf JNK inhibitor
RGKRKALKLF

SEQ ID NO: 26 - protein, 10 aa, Artificial
  rGKRKALrLf JNK inhibitor
RGKRKALRLF

SEQ ID NO: 27 - protein, 10 aa, Artificial
  RRrRKALrLf JNK inhibitor
RRRRKALRLF

SEQ ID NO: 28 - protein, 9 aa, Artificial Sequence
  Description of artificial sequence generic subformula (Ib) DlLLLxDmLLLyDn
XXXXXXXXX

SEQ ID NO: 29 - protein, 9 aa, Artificial Sequence
  Description of artificial sequence generic subformula (Ie) DLLLD(LLLD)a
XXXXXXXXX

SEQ ID NO: 30 - protein, 9 aa, Artificial Sequence
  Description of artificial sequence generic subformula (If) DLLLDLLLD
XXXXXXXXX

SEQ ID NO: 31 - protein, 9 aa, Artificial
  Description of sequence consensus sequence rXXXrXXXr
RXXXRXXXR

SEQ ID NO: 32 - protein, 9 aa, Artificial
  r3 (generic; right half)
RKKRRXXXR

SEQ ID NO: 33 - protein, 9 aa, Artificial
  r3 (generic; left half)
RXXXRQRRR

SEQ ID NO: 34 - protein, 9 aa, Artificial
  r3 (generic; individual)
RXXXRXXXR

SEQ ID NO: 35 - protein, 86 aa, Human immunodeficiency virus type 1
  misc_feature: Description of sequence  HIV-1 TAT sequence (aa 1-86)
MEPVDPRLEPWKHPGSQPKTACTNCYCKKCCFHCQVCFITKALGISYGRKKRRQRRRPPQGSQTHQVSLSKQPTSQSRGDPTGPKE

SEQ ID NO: 36 - protein, 36 aa, Human immunodeficiency virus type 1
  misc_feature: Description of sequence  HIV-1 TAT sequence (aa 37-72)
CFITKALGISYGRKKRRQRRRPPQGSQTHQVSLSKQ

SEQ ID NO: 37 - protein, 22 aa, Human immunodeficiency virus type 1
  misc_feature: Description of sequence  HIV-1 TAT sequence (aa 37-58)
CFITKALGISYGRKKRRQRRRP

SEQ ID NO: 38 - protein, 24 aa, Human immunodeficiency virus type 1
  misc_feature: Description of sequence  HIV-1 TAT sequence (aa 38-58) including an additional N-terminal GCC
FITKALGISYGRKKRRQRRRPGGC

SEQ ID NO: 39 - protein, 15 aa, Human immunodeficiency virus type 1
  misc_feature: Description of sequence  HIV-1 TAT sequence (aa 47-58) including an additional C-terminal GCC
CGGYGRKKRRQRRRP

SEQ ID NO: 40 - protein, 15 aa, Human immunodeficiency virus type 1
  misc_feature: Description of sequence  HIV-1 TAT sequence (aa 47-58) including an additional N-terminal GCC
YGRKKRRQRRRPGGC

SEQ ID NO: 41 - protein, 56 aa, Human immunodeficiency virus type 1
  misc_feature: Description of sequence  HIV-1 TAT sequence (aa 1-72) including a mutated Cys to Ala residue at position 37
MEPVDPRLEPWKHPGSQPKTAFITKALGISYGRKKRRQRRRPPQGSQTHQVSLSKQ

SEQ ID NO: 42 - protein, 10 aa, Artificial
  Description of sequence  trafficking sequence L-TAT (s1a)
GRKKRRQRRR

SEQ ID NO: 43 - protein, 9 aa, Artificial Sequence
  Description of sequence  trafficking sequence L-TAT (s1b)
RKKRRQRRR

SEQ ID NO: 44 - protein, 11 aa, Artificial Sequence
  Description of sequence  trafficking sequence L-TAT (s1c)
YDRKKRRQRRR

SEQ ID NO: 45 - protein, 9 aa, Artificial Sequence
  Description of artificial sequence D-TAT
RRRQRRKKR

SEQ ID NO: 46 - protein, 9 aa, Artificial Sequence
  trafficking sequence r3-L-TAT
RKKRRQRRR

SEQ ID NO: 47 - protein, 9 aa, Artificial Sequence
  trafficking sequence r3-L-TATi
RRRQRRKKR

SEQ ID NO: 48 - protein, 9 aa, Artificial Sequence
  Description of sequence  trafficking sequence betaA-r3-L-TAT
RKKRRQRRR

SEQ ID NO: 49 - protein, 9 aa, Artificial Sequence
  Description of sequence  trafficking sequence betaA-r3-L-TAT
RRRQRRKKR

SEQ ID NO: 50 - protein, 9 aa, Artificial Sequence
  Description of sequence  trafficking sequence FITC-betaA-r3-L-TAT
RKKRRQRRR

SEQ ID NO: 51 - protein, 9 aa, Artificial Sequence
  Description of sequence  trafficking sequence FITC-betaA-r3-L-TAT
RRRQRRKKR

SEQ ID NO: 52 - protein, 9 aa, Artificial Sequence
  Description of sequence  trafficking sequence TAT(s2-1)
RAKRRQRRR

SEQ ID NO: 53 - protein, 9 aa, Artificial Sequence
  Description of sequence  trafficking sequence TAT(s2-2)
RKARRQRRR

SEQ ID NO: 54 - protein, 9 aa, Artificial Sequence
  Description of sequence  trafficking sequence TAT(s2-3)
RKKARQRRR

SEQ ID NO: 55 - protein, 9 aa, Artificial Sequence
  Description of sequence  trafficking sequence TAT(s2-4)
RKKRRARRR

SEQ ID NO: 56 - protein, 9 aa, Artificial Sequence
  Description of sequence  trafficking sequence TAT(s2-5)
RKKRRQARR

SEQ ID NO: 57 - protein, 9 aa, Artificial Sequence
  Description of sequence  trafficking sequence TAT(s2-6))
RKKRRQRAR

SEQ ID NO: 58 - protein, 9 aa, Artificial Sequence
  Description of sequence  trafficking sequence TAT(s2-7)
RDKRRQRRR

SEQ ID NO: 59 - protein, 9 aa, Artificial Sequence
  trafficking sequence TAT(s2-8)
RKDRRQRRR

SEQ ID NO: 60 - protein, 9 aa, Artificial Sequence
  trafficking sequence TAT(s2-9)
RKKDRQRRR

SEQ ID NO: 61 - protein, 9 aa, Artificial Sequence
  trafficking sequence TAT(s2-10)
RKKRRDRRR

SEQ ID NO: 62 - protein, 9 aa, Artificial Sequence
  trafficking sequence TAT(s2-11)
RKKRRQDRR

SEQ ID NO: 63 - protein, 9 aa, Artificial Sequence
  trafficking sequence TAT(s2-12)
RKKRRQRDR

SEQ ID NO: 64 - protein, 9 aa, Artificial Sequence
  trafficking sequence TAT(s2-13)
REKRRQRRR

SEQ ID NO: 65 - protein, 9 aa, Artificial Sequence
  trafficking sequence TAT(s2-14)
RKERRQRRR

SEQ ID NO: 66 - protein, 9 aa, Artificial Sequence
  trafficking sequence TAT(s2-15)
RKKERQRRR

SEQ ID NO: 67 - protein, 9 aa, Artificial Sequence
  trafficking sequence TAT(s2-16)
RKKRRERRR

SEQ ID NO: 68 - protein, 9 aa, Artificial Sequence
  trafficking sequence TAT(s2-17)
RKKRRQERR

SEQ ID NO: 69 - protein, 9 aa, Artificial Sequence
  trafficking sequence TAT(s2-18)
RKKRRQRER

SEQ ID NO: 70 - protein, 9 aa, Artificial Sequence
  trafficking sequence TAT(s2-19)
RFKRRQRRR

SEQ ID NO: 71 - protein, 9 aa, Artificial Sequence
  trafficking sequence TAT(s2-20)
RKFRRQRRR

SEQ ID NO: 72 - protein, 9 aa, Artificial Sequence
  trafficking sequence TAT(s2-21)
RKKFRQRRR

SEQ ID NO: 73 - protein, 9 aa, Artificial Sequence
  trafficking sequence TAT(s2-22)
RKKRRFRRR

SEQ ID NO: 74 - protein, 9 aa, Artificial Sequence
  trafficking sequence TAT(s2-23)
RKKRRQFRR

SEQ ID NO: 75 - protein, 9 aa, Artificial Sequence
  trafficking sequence TAT(s2-24)
RKKRRQRFR

SEQ ID NO: 76 - protein, 9 aa, Artificial Sequence
  trafficking sequence TAT(s2-25)
RRKRRQRRR

SEQ ID NO: 77 - protein, 9 aa, Artificial Sequence
  trafficking sequence TAT(s2-26)
RKRRRQRRR

SEQ ID NO: 78 - protein, 9 aa, Artificial Sequence
  trafficking sequence TAT(s2-27)
RKKKRQRRR

SEQ ID NO: 79 - protein, 9 aa, Artificial Sequence
  trafficking sequence TAT(s2-28)
RKKRRRRRR

SEQ ID NO: 80 - protein, 9 aa, Artificial Sequence
  trafficking sequence TAT(s2-29)
RKKRRQKRR

SEQ ID NO: 81 - protein, 9 aa, Artificial Sequence
  trafficking sequence TAT(s2-30)
RKKRRQRKR

SEQ ID NO: 82 - protein, 9 aa, Artificial Sequence
  trafficking sequence TAT(s2-31)
RHKRRQRRR

SEQ ID NO: 83 - protein, 9 aa, Artificial Sequence
  trafficking sequence TAT(s2-32)
RKHRRQRRR

SEQ ID NO: 84 - protein, 9 aa, Artificial Sequence
  trafficking sequence TAT(s2-33)
RKKHRQRRR

SEQ ID NO: 85 - protein, 9 aa, Artificial Sequence
  trafficking sequence TAT(s2-34)
RKKRRHRRR

SEQ ID NO: 86 - protein, 9 aa, Artificial Sequence
  trafficking sequence TAT(s2-35)
RKKRRQHRR

SEQ ID NO: 87 - protein, 9 aa, Artificial Sequence
  trafficking sequence TAT(s2-36)
RKKRRQRHR

SEQ ID NO: 88 - protein, 9 aa, Artificial Sequence
  trafficking sequence TAT(s2-37)
RIKRRQRRR

SEQ ID NO: 89 - protein, 9 aa, Artificial Sequence
  trafficking sequence TAT(s2-38)
RKIRRQRRR

SEQ ID NO: 90 - protein, 9 aa, Artificial Sequence
  trafficking sequence TAT(s2-39)
RKKIRQRRR

SEQ ID NO: 91 - protein, 9 aa, Artificial Sequence
  trafficking sequence TAT(s2-40)
RKKRRIRRR

SEQ ID NO: 92 - protein, 9 aa, Artificial Sequence
  trafficking sequence TAT(s2-41)
RKKRRQIRR

SEQ ID NO: 93 - protein, 9 aa, Artificial Sequence
  trafficking sequence TAT(s2-42)
RKKRRQRIR

SEQ ID NO: 94 - protein, 9 aa, Artificial Sequence
  trafficking sequence TAT(s2-43)
RLKRRQRRR

SEQ ID NO: 95 - protein, 9 aa, Artificial Sequence
  trafficking sequence TAT(s2-44)
RKLRRQRRR

SEQ ID NO: 96 - protein, 9 aa, Artificial Sequence
  trafficking sequence TAT(s2-45)
RKKLRQRRR

SEQ ID NO: 97 - protein, 9 aa, Artificial Sequence
  trafficking sequence TAT(s2-46)
RKKRRLRRR

SEQ ID NO: 98 - protein, 9 aa, Artificial Sequence
  trafficking sequence TAT(s2-47)
RKKRRQLRR

SEQ ID NO: 99 - protein, 9 aa, Artificial Sequence
  trafficking sequence TAT(s2-48)
RKKRRQRLR

SEQ ID NO: 100 - protein, 9 aa, Artificial Sequence
  trafficking sequence TAT(s2-49)
RMKRRQRRR

SEQ ID NO: 101 - protein, 9 aa, Artificial Sequence
  trafficking sequence TAT(s2-50)
RKMRRQRRR

SEQ ID NO: 102 - protein, 9 aa, Artificial Sequence
  trafficking sequence TAT(s2-51)
RKKMRQRRR

SEQ ID NO: 103 - protein, 9 aa, Artificial Sequence
  trafficking sequence TAT(s2-52)
RKKRRMRRR

SEQ ID NO: 104 - protein, 9 aa, Artificial Sequence
  trafficking sequence TAT(s2-53)
RKKRRQMRR

SEQ ID NO: 105 - protein, 9 aa, Artificial Sequence
  trafficking sequence TAT(s2-54)
RKKRRQRMR

SEQ ID NO: 106 - protein, 9 aa, Artificial Sequence
  trafficking sequence TAT(s2-55)
RNKRRQRRR

SEQ ID NO: 107 - protein, 9 aa, Artificial Sequence
  trafficking sequence TAT(s2-56)
RKNRRQRRR

SEQ ID NO: 108 - protein, 9 aa, Artificial Sequence
  trafficking sequence TAT(s2-57)
RKKNRQRRR

SEQ ID NO: 109 - protein, 9 aa, Artificial Sequence
  trafficking sequence TAT(s2-58)
RKKRRNRRR

SEQ ID NO: 110 - protein, 9 aa, Artificial Sequence
  trafficking sequence TAT(s2-59)
RKKRRQNRR

SEQ ID NO: 111 - protein, 9 aa, Artificial Sequence
  trafficking sequence TAT(s2-60)
RKKRRQRNR

SEQ ID NO: 112 - protein, 9 aa, Artificial Sequence
  trafficking sequence TAT(s2-61)
RQKRRQRRR

SEQ ID NO: 113 - protein, 9 aa, Artificial Sequence
  trafficking sequence TAT(s2-62)
RKQRRQRRR

SEQ ID NO: 114 - protein, 9 aa, Artificial Sequence
  trafficking sequence TAT(s2-63)
RKKQRQRRR

SEQ ID NO: 115 - protein, 9 aa, Artificial Sequence
  trafficking sequence TAT(s2-64)
RKKRRKRRR

SEQ ID NO: 116 - protein, 9 aa, Artificial Sequence
  trafficking sequence TAT(s2-65)
RKKRRQQRR

SEQ ID NO: 117 - protein, 9 aa, Artificial Sequence
  trafficking sequence TAT(s2-66)
RKKRRQRQR

SEQ ID NO: 118 - protein, 9 aa, Artificial Sequence
  trafficking sequence TAT(s2-67)
RSKRRQRRR

SEQ ID NO: 119 - protein, 9 aa, Artificial Sequence
  trafficking sequence TAT(s2-68)
RKSRRQRRR

SEQ ID NO: 120 - protein, 9 aa, Artificial Sequence
  trafficking sequence TAT(s2-69)
RKKSRQRRR

SEQ ID NO: 121 - protein, 9 aa, Artificial Sequence
  trafficking sequence TAT(s2-70)
RKKRRSRRR

SEQ ID NO: 122 - protein, 9 aa, Artificial Sequence
  trafficking sequence TAT(s2-71)
RKKRRQSRR

SEQ ID NO: 123 - protein, 9 aa, Artificial Sequence
  trafficking sequence TAT(s2-72)
RKKRRQRSR

SEQ ID NO: 124 - protein, 9 aa, Artificial Sequence
  trafficking sequence TAT(s2-73)
RTKRRQRRR

SEQ ID NO: 125 - protein, 9 aa, Artificial Sequence
  trafficking sequence TAT(s2-74)
RKTRRQRRR

SEQ ID NO: 126 - protein, 9 aa, Artificial Sequence
  trafficking sequence TAT(s2-75)
RKKTRQRRR

SEQ ID NO: 127 - protein, 9 aa, Artificial Sequence
  trafficking sequence TAT(s2-76)
RKKRRTRRR

SEQ ID NO: 128 - protein, 9 aa, Artificial Sequence
  trafficking sequence TAT(s2-77)
RKKRRQTRR

SEQ ID NO: 129 - protein, 9 aa, Artificial Sequence
  trafficking sequence TAT(s2-78)
RKKRRQRTR

SEQ ID NO: 130 - protein, 9 aa, Artificial Sequence
  trafficking sequence TAT(s2-79)
RVKRRQRRR

SEQ ID NO: 131 - protein, 9 aa, Artificial Sequence
  trafficking sequence TAT(s2-80)
RKVRRQRRR

SEQ ID NO: 132 - protein, 9 aa, Artificial Sequence
  trafficking sequence TAT(s2-81)
RKKVRQRRR

SEQ ID NO: 133 - protein, 9 aa, Artificial Sequence
  trafficking sequence TAT(s2-82)
RKKRRVRRR

SEQ ID NO: 134 - protein, 9 aa, Artificial Sequence
  trafficking sequence TAT(s2-83)
RKKRRQVRR

SEQ ID NO: 135 - protein, 9 aa, Artificial Sequence
  trafficking sequence TAT(s2-84)
RKKRRQRVR

SEQ ID NO: 136 - protein, 9 aa, Artificial Sequence
  trafficking sequence TAT(s2-85)
RWKRRQRRR

SEQ ID NO: 137 - protein, 9 aa, Artificial Sequence
  trafficking sequence TAT(s2-86)
RKWRRQRRR

SEQ ID NO: 138 - protein, 9 aa, Artificial Sequence
  trafficking sequence TAT(s2-87)
RKKWRQRRR

SEQ ID NO: 139 - protein, 9 aa, Artificial Sequence
  trafficking sequence TAT(s2-88)
RKKRRWRRR

SEQ ID NO: 140 - protein, 9 aa, Artificial Sequence
  trafficking sequence TAT(s2-89)
RKKRRQWRR

SEQ ID NO: 141 - protein, 9 aa, Artificial Sequence
  trafficking sequence TAT(s2-90)
RKKRRQRWR

SEQ ID NO: 142 - protein, 9 aa, Artificial Sequence
  trafficking sequence TAT(s2-91)
RYKRRQRRR

SEQ ID NO: 143 - protein, 9 aa, Artificial Sequence
  trafficking sequence TAT(s2-92)
RKYRRQRRR

SEQ ID NO: 144 - protein, 9 aa, Artificial Sequence
  trafficking sequence TAT(s2-93)
RKKYRQRRR

SEQ ID NO: 145 - protein, 9 aa, Artificial Sequence
  trafficking sequence TAT(s2-94)
RKKRRYRRR

SEQ ID NO: 146 - protein, 9 aa, Artificial Sequence
  trafficking sequence TAT(s2-95)
RKKRRQYRR

SEQ ID NO: 147 - protein, 9 aa, Artificial Sequence
  Trafficking sequence TAT(s2-96)
RKKRRQRYR

SEQ ID NO: 148 - protein, 8 aa, Artificial
  Trafficking sequence TAT(s2-97)
RKKRRQRR

SEQ ID NO: 149 - protein, 9 aa, Artificial
  Trafficking sequence TAT(s2-98)
RKKRRQRRK

SEQ ID NO: 150 - protein, 9 aa, Artificial
  Trafficking sequence TAT(s2-99)
RKKRRQRRR

SEQ ID NO: 151 - protein, 9 aa, Artificial Sequence
  trafficking sequence r3R6
RRRRRRRRR

SEQ ID NO: 152 - protein, 9 aa, Artificial Sequence
  trafficking sequence L-R9
RRRRRRRRR

SEQ ID NO: 153 - protein, 8 aa, Artificial Sequence
  trafficking sequence L-R8
RRRRRRRR

SEQ ID NO: 154 - protein, 7 aa, Artificial Sequence
  trafficking sequence L-R7
RRRRRRR

SEQ ID NO: 155 - protein, 6 aa, Artificial Sequence
  trafficking sequence L-R6
RRRRRR

SEQ ID NO: 156 - protein, 5 aa, Artificial Sequence
  trafficking sequence L-R5
RRRRR

SEQ ID NO: 157 - protein, 9 aa, Artificial
  all D transporter construct (all amino acid residues are D-amino acids)
RRRRRRRRR

SEQ ID NO: 158 - protein, 9 aa, Artificial
  Description of sequence D/L transporter construct (D and L amino acid residues alternate, beginning wit D amino acids)
RRRRRRRRR

SEQ ID NO: 159 - protein, 9 aa, Artificial
  Description of sequence DD/LL transporter construct
RRRRRRRRR

SEQ ID NO: 160 - protein, 11 aa, Artificial Sequence
  trafficking sequence PTD-4
YARAAARQARA

SEQ ID NO: 161 - protein, 11 aa, Artificial Sequence
  trafficking sequence PTD-4
WARAAARQARA

SEQ ID NO: 162 - protein, 11 aa, Artificial Sequence
  trafficking sequence PTD-4
WARAQRAAARA

SEQ ID NO: 163 - protein, 16 aa, Artificial Sequence
  trafficking sequence L-P1 (Penetratin)
RQVKVWFQNRRMKWKK

SEQ ID NO: 164 - protein, 16 aa, Artificial Sequence
  trafficking sequence D-P1 (Penetratin)
KKWKMRRNQFWVKVQR

SEQ ID NO: 165 - protein, 17 aa, Artificial Sequence
  trafficking sequence JNK1, bestfit
WKRAAARKARAMSLNLF

SEQ ID NO: 166 - protein, 17 aa, Artificial Sequence
  trafficking sequence JNK1, bestfit (variant 1)
WKRAAARAARAMSLNLF

SEQ ID NO: 167 - protein, 9 aa, Artificial Sequence
  trafficking sequence MDCK transcytose sequence
RYRGDLGRR

SEQ ID NO: 168 - protein, 4 aa, Artificial Sequence
  trafficking sequence YKGL
YKGL

SEQ ID NO: 169 - protein, 4 aa, Artificial
  trafficking sequence RRTK
RRTK

SEQ ID NO: 170 - protein, 4 aa, Artificial
  trafficking sequence RRPK
RRPK

SEQ ID NO: 171 - protein, 20 aa, Artificial
  rKKRrQRRrRPkRPTTLNLf JNK inhibitor
RKKRRQRRRRPKRPTTLNLF

SEQ ID NO: 172 - protein, 20 aa, Artificial
  rKKRrQRRrRPkRPaTLNLf  JNK inhibitor
RKKRRQRRRRPKRPATLNLF

SEQ ID NO: 173 - protein, 20 aa, Artificial
  rKKRrQRRrRPkRPTTLrLf JNK inhibitor
RKKRRQRRRRPKRPTTLRLF

SEQ ID NO: 174 - protein, 17 aa, Artificial
  rKKRrQRRrRPTTLNLf JNK inhibitor
RKKRRQRRRRPTTLNLF

SEQ ID NO: 175 - protein, 16 aa, Artificial
  rKKRrQRrRPTTLNLf JNK inhibitor
RKKRRQRRRPTTLNLF

SEQ ID NO: 176 - protein, 20 aa, Artificial
  rKKRrQRRrRPkRPTTLNLw JNK inhibitor
RKKRRQRRRRPKRPTTLNLW

SEQ ID NO: 177 - protein, 20 aa, Artificial
  rKKRrQRRrRPkRPTDLNLf JNK inhibitor
RKKRRQRRRRPKRPTDLNLF

SEQ ID NO: 178 - protein, 17 aa, Artificial
  rKKRrQRRrRPTTLrLw JNK inhibitor
RKKRRQRRRRPTTLRLW

SEQ ID NO: 179 - protein, 16 aa, Artificial
  rKKRrQRrRPTTLrLw JNK inhibitor
RKKRRQRRRPTTLRLW

SEQ ID NO: 180 - protein, 17 aa, Artificial
  rKKRrQRRrRPTDLrLw JNK inhibitor
RKKRRQRRRRPTDLRLW

SEQ ID NO: 181 - protein, 16 aa, Artificial
  rKKRrQRrRPTDLrLw JNK inhibitor
RKKRRQRRRPTDLRLW

SEQ ID NO: 182 - protein, 17 aa, Artificial
  rKKRrQRRrRPaTLNLf JNK inhibitor
RKKRRQRRRRPATLNLF

SEQ ID NO: 183 - protein, 16 aa, Artificial
  rKKRrQRrRPaTLNLf JNK inhibitor
RKKRRQRRRPATLNLF

SEQ ID NO: 184 - protein, 17 aa, Artificial
  rKKRrQRrKRPaTLNLf JNK inhibitor
RKKRRQRRKRPATLNLF

SEQ ID NO: 185 - protein, 20 aa, Artificial
  rKKRrQRRrRPkRPsTLNLf JNK inhibitor
RKKRRQRRRRPKRPSTLNLF

SEQ ID NO: 186 - protein, 20 aa, Artificial
  rKKRrQRRrRPkRPqTLNLf JNK inhibitor
RKKRRQRRRRPKRPQTLNLF

SEQ ID NO: 187 - protein, 20 aa, Artificial
  rKKRrQRRrRPkRPkTLNLf JNK inhibitor
RKKRRQRRRRPKRPKTLNLF

SEQ ID NO: 188 - protein, 18 aa, Artificial
  rKKRrQRRrGKRKALKLf JNK inhibitor
RKKRRQRRRGKRKALKLF

SEQ ID NO: 189 - protein, 18 aa, Artificial
  rKKRrQRRrGKRKALrLf JNK inhibitor
RKKRRQRRRGKRKALRLF

SEQ ID NO: 190 - protein, 16 aa, Artificial
  rKKRrQRRrRKALrLf  JNK inhibitor
RKKRRQRRRRKALRLF

SEQ ID NO: 191 - protein, 8 aa, Artificial
  RPTTLNLF JNK inhibitor
RPTTLNLF

SEQ ID NO: 192 - protein, 9 aa, Artificial
  KRPTTLNLF JNK inhibitor
KRPTTLNLF

SEQ ID NO: 193 - protein, 11 aa, Artificial
  L-IB1(s24)
RPKRPTTLNLF

SEQ ID NO: 194 - protein, 29 aa, Artificial
  GRKKRRQRRRPPKRPTTLNLFPQVPRSQD JNK inhibitor
GRKKRRQRRRPPKRPTTLNLFPQVPRSQD

SEQ ID NO: 195 - protein, 25 aa, Artificial
  GRKKRRQRRRPTTLNLFPQVPRSQD JNK inhibitor
GRKKRRQRRRPTTLNLFPQVPRSQD

SEQ ID NO: 196 - protein, 31 aa, Artificial
  L-TAT-IB1
GRKKRRQRRRPPRPKRPTTLNLFPQVPRSQD

SEQ ID NO: 197 - protein, 31 aa, Artificial
  D-TAT-IB1
DQSRPVQPFLNLTTPRKPRPPRRRQRRKKRG

SEQ ID NO: 198 - protein, 39 aa, Artificial
  cJun (29-67)
SNPKILKQSMTLNLADPVGSLKPHLRAKNSDLLTSPDVG

SEQ ID NO: 199 - protein, 20 aa, Artificial
  RKKRRQRRRRPKRPATLNLF antibody negative control
RKKRRQRRRRPKRPATLNLF

SEQ ID NO: 200 - protein, 20 aa, Artificial
  rKKRrQRRrR PkAAaAANAf     JNK inhibitor
RKKRRQRRRRPKAAAAANAF